SEQUENCE LISTING (687 sequences)

SEQ ID NO: 1 - DNA, 85 bp, Artificial Sequence
  Synthetic oligonucleotide
aaataatcaggagaaggagatggcatgtttgttggtgattccaaggagctctctcagtcatgatcctgtacatttgctctgcctt

SEQ ID NO: 2 - DNA, 87 bp, Artificial Sequence
  Synthetic oligonucleotide
ggagcagcgtctctgccatcgtcctcgtccatgtcctggtcacacttccagattaactacttcgaacctgtacatttgctctgcctt

SEQ ID NO: 3 - DNA, 81 bp, Artificial Sequence
  Synthetic oligonucleotide
aggtaaatatttaccacctcttggtgtttattttaccgtctatatacaagagcaccgcaacctgtacatttgctctgcctt

SEQ ID NO: 4 - DNA, 83 bp, Artificial Sequence
  Synthetic oligonucleotide
ctttcagtcagatgtatatgcatttgggattgttctgtatgaattgatgagtatgtgcggttcctgtacatttgctctgcctt

SEQ ID NO: 5 - DNA, 85 bp, Artificial Sequence
  Synthetic oligonucleotide
tatctgctaagaaacagacatccatatacagagatgaaaatgatgatttttgatacatcaactgcctgtacatttgctctgcctt

SEQ ID NO: 6 - DNA, 85 bp, Artificial Sequence
  Synthetic oligonucleotide
atcttgccttgccttccaccctaataccagcataatctactaaagcttctctacaactgatacgcctgtacatttgctctgcctt

SEQ ID NO: 7 - DNA, 84 bp, Artificial Sequence
  Synthetic oligonucleotide
tgtccagtgatatggttataatgtgaaacaaaactcacctgggtcacttttactgcagaccttcctgtacatttgctctgcctt

SEQ ID NO: 8 - DNA, 83 bp, Artificial Sequence
  Synthetic oligonucleotide
attaaaggcgccgccgggcggctcccgctgccatcctggctctcctgcgcactgagagataacctgtacatttgctctgcctt

SEQ ID NO: 9 - DNA, 87 bp, Artificial Sequence
  Synthetic oligonucleotide
gatttgcgttctgcactatgacataatttggccttcagggtgagttgttttccgatatatatgtctcctgtacatttgctctgcctt

SEQ ID NO: 10 - DNA, 84 bp, Artificial Sequence
  Synthetic oligonucleotide
agacttcaccttgtgatctgcagggactgaccttagtgttgttgtgttggcaatagtcttggacctgtacatttgctctgcctt

SEQ ID NO: 11 - DNA, 88 bp, Artificial Sequence
  Synthetic oligonucleotide
agagatctccaaagacactccacggaatgagggcttgttgcccttgtctttatgttaatatcaacagcctgtacatttgctctgcctt

SEQ ID NO: 12 - DNA, 83 bp, Artificial Sequence
  Synthetic oligonucleotide
cctatacaattgagatgttgggggaaccacaacataactcattcagactgtgacgagcttgacctgtacatttgctctgcctt

SEQ ID NO: 13 - DNA, 83 bp, Artificial Sequence
  Synthetic oligonucleotide
tttttattgtatatgcatgcacatcccaaggaccaagagaccagctacaccgcatgtgacgacctgtacatttgctctgcctt

SEQ ID NO: 14 - DNA, 84 bp, Artificial Sequence
  Synthetic oligonucleotide
gcaggcatcaaagtgcaggacgtccggctgaatggctccgcagctggccaaagctgccattcccctgtacatttgctctgcctt

SEQ ID NO: 15 - DNA, 85 bp, Artificial Sequence
  Synthetic oligonucleotide
tctgcattccctgtcactgcgtcactggccttcagacagagccaaggtgcttgattgctacaatcctgtacatttgctctgcctt

SEQ ID NO: 16 - DNA, 83 bp, Artificial Sequence
  Synthetic oligonucleotide
gcccaagtgtttctctggcatctgttggtgtctggatccaccactctactggtgcttcgtcacctgtacatttgctctgcctt

SEQ ID NO: 17 - DNA, 87 bp, Artificial Sequence
  Synthetic oligonucleotide
gttcagatttcactgcctcatgttgatatttctttccagatgacgactgtttaatctctcacacctcctgtacatttgctctgcctt

SEQ ID NO: 18 - DNA, 86 bp, Artificial Sequence
  Synthetic oligonucleotide
tagacacattgtcatcatggacaggcggtggatacgtgccgtctagctgatacctaactatagaacctgtacatttgctctgcctt

SEQ ID NO: 19 - DNA, 85 bp, Artificial Sequence
  Synthetic oligonucleotide
gcttgtctttgaaacttcttggcaaatcggttaagatctgggaatcgacggttcatacctcaatcctgtacatttgctctgcctt

SEQ ID NO: 20 - DNA, 82 bp, Artificial Sequence
  Synthetic oligonucleotide
tacctcccatattggggcctacagaacaaattatatcagaaagcaagattgataccgttgacctgtacatttgctctgcctt

SEQ ID NO: 21 - DNA, 82 bp, Artificial Sequence
  Synthetic oligonucleotide
tgccaatgaccacagtgtcgggccccgcatccagtgacgagggcgtggtgggagaaccagacctgtacatttgctctgcctt

SEQ ID NO: 22 - DNA, 83 bp, Artificial Sequence
  Synthetic oligonucleotide
atcccccttaaaatcacgctcacttgccgcgcataggccatctcgatgttgacgacgaacagcctgtacatttgctctgcctt

SEQ ID NO: 23 - DNA, 86 bp, Artificial Sequence
  Synthetic oligonucleotide
ggggaggtgaagctgtctatctcctacaaaaacaataaactcttcatcatattaacgtcaataaacctgtacatttgctctgcctt

SEQ ID NO: 24 - DNA, 87 bp, Artificial Sequence
  Synthetic oligonucleotide
cgagatttttttccttccaatatattctacataagttcccggaaagtcccagaatggatctaaaagcctgtacatttgctctgcctt

SEQ ID NO: 25 - DNA, 82 bp, Artificial Sequence
  Synthetic oligonucleotide
cttttccttaaaaagaaaaagaaagggagtcattaagcaaccacgtatcaccgatcattgtcctgtacatttgctctgcctt

SEQ ID NO: 26 - DNA, 85 bp, Artificial Sequence
  Synthetic oligonucleotide
tgtgtgttatgatttctgttgcagagttgtgaaaaccgtggctgtgaaaacaccagaggtcttccctgtacatttgctctgcctt

SEQ ID NO: 27 - DNA, 84 bp, Artificial Sequence
  Synthetic oligonucleotide
ggccctgcaaatgccctcatcagaagccccgttgccctcctggagtgagcactcgaagatcgacctgtacatttgctctgcctt

SEQ ID NO: 28 - DNA, 84 bp, Artificial Sequence
  Synthetic oligonucleotide
gctactccaggcacacgccgcacatcctgcaggcagagagtaagaggaagtcgatacagcctacctgtacatttgctctgcctt

SEQ ID NO: 29 - DNA, 83 bp, Artificial Sequence
  Synthetic oligonucleotide
gatctctcctgagtcctcactaacaacagggggtagatttattgttttcaagccaagtatcacctgtacatttgctctgcctt

SEQ ID NO: 30 - DNA, 81 bp, Artificial Sequence
  Synthetic oligonucleotide
ccactagccctggttcaggtcagggatgccatgtcgtcggggcccaggcaaatcttttcgcctgtacatttgctctgcctt

SEQ ID NO: 31 - DNA, 84 bp, Artificial Sequence
  Synthetic oligonucleotide
tggcagcctcactgtgcggagcatggagccacacatggtgtaggcacagttaagttatttatacctgtacatttgctctgcctt

SEQ ID NO: 32 - DNA, 83 bp, Artificial Sequence
  Synthetic oligonucleotide
aagtaccccaaagtgtgagggccttccctctgccgcacatcatcgagaacggtccggtcgctcctgtacatttgctctgcctt

SEQ ID NO: 33 - DNA, 85 bp, Artificial Sequence
  Synthetic oligonucleotide
gtgcttctgaaactgttatcttcccaggagcaatctggggatagaaagcatattagtgtggagacctgtacatttgctctgcctt

SEQ ID NO: 34 - DNA, 86 bp, Artificial Sequence
  Synthetic oligonucleotide
gggccagtctttaaatgcttcctggaaaatgttactacctatgaaataaacttctggcagtggaccctgtacatttgctctgcctt

SEQ ID NO: 35 - DNA, 84 bp, Artificial Sequence
  Synthetic oligonucleotide
aaggaagcagcgtgcagtgccattccttcctccacgtaagcctcgttaggatccagtcaacttcctgtacatttgctctgcctt

SEQ ID NO: 36 - DNA, 84 bp, Artificial Sequence
  Synthetic oligonucleotide
attgggggtatactggaaaagtatttttggtgttgaagtttgggctgtgagcgagccttgtcacctgtacatttgctctgcctt

SEQ ID NO: 37 - DNA, 82 bp, Artificial Sequence
  Synthetic oligonucleotide
gggatgtttcttgtcctcgctcaagacagaattcgagagtgagccagtggcggtggaaggacctgtacatttgctctgcctt

SEQ ID NO: 38 - DNA, 84 bp, Artificial Sequence
  Synthetic oligonucleotide
tttgacaccaataaaacggagtgccactgaagggttttgagctgaggagagtccagtgaactacctgtacatttgctctgcctt

SEQ ID NO: 39 - DNA, 84 bp, Artificial Sequence
  Synthetic oligonucleotide
accgtacctctccccgacgtgggcaggcgtgagttgtcagtttagagaacaacataatcgtagcctgtacatttgctctgcctt

SEQ ID NO: 40 - DNA, 83 bp, Artificial Sequence
  Synthetic oligonucleotide
ctgcttctagggttgggatctcccagggaagaccgggcttgccatgtgcaacgaggccttctcctgtacatttgctctgcctt

SEQ ID NO: 41 - DNA, 88 bp, Artificial Sequence
  Synthetic oligonucleotide
gtcattttgctgtttgttttctatatgcagtataacatttttgtctcttaatatgtaatcagctgatcctgtacatttgctctgcctt

SEQ ID NO: 42 - DNA, 83 bp, Artificial Sequence
  Synthetic oligonucleotide
taggagacagagaatgttctgtgggaccacaaccgagacagaagagctctaatgccggccgccctgtacatttgctctgcctt

SEQ ID NO: 43 - DNA, 85 bp, Artificial Sequence
  Synthetic oligonucleotide
cctgtaacacacgcccacaggggcttcaggaactataaacattcacccttttctcttggatatgcctgtacatttgctctgcctt

SEQ ID NO: 44 - DNA, 83 bp, Artificial Sequence
  Synthetic oligonucleotide
tatttgataaattaaccctagaacaactatctgcactcagaaacacatacggaacgcattagcctgtacatttgctctgcctt

SEQ ID NO: 45 - DNA, 85 bp, Artificial Sequence
  Synthetic oligonucleotide
gagcatcctgaagcaattctgtttgtaatcctggaagtagtaactgggaacctatactcaccagcctgtacatttgctctgcctt

SEQ ID NO: 46 - DNA, 85 bp, Artificial Sequence
  Synthetic oligonucleotide
tgaattatttttcttccccttcatttttgtttaagctctattgttttttgtaccattcgagccacctgtacatttgctctgcctt

SEQ ID NO: 47 - DNA, 86 bp, Artificial Sequence
  Synthetic oligonucleotide
agaacaatgtccacatgtttcctctgtgccattactaagatggtccccttaccattggcggaagccctgtacatttgctctgcctt

SEQ ID NO: 48 - DNA, 84 bp, Artificial Sequence
  Synthetic oligonucleotide
catcccaccctgtctcactggagccaggatccatgaggtcccgtgagctaggcttcagaaggccctgtacatttgctctgcctt

SEQ ID NO: 49 - DNA, 82 bp, Artificial Sequence
  Synthetic oligonucleotide
tccatcctaaaggacttacagtttcttagaataacatggagtgatttttcggagtgactgtcctgtacatttgctctgcctt

SEQ ID NO: 50 - DNA, 85 bp, Artificial Sequence
  Synthetic oligonucleotide
gaaaacagtcaaaatggctgtcgacaatgaaatggatacatcagttgagactcaggcaatgtgacctgtacatttgctctgcctt

SEQ ID NO: 51 - DNA, 84 bp, Artificial Sequence
  Synthetic oligonucleotide
gaaagactaataattttgcccatgatcacctcaccatctcactctgaaatggcggctctcaggcctgtacatttgctctgcctt

SEQ ID NO: 52 - DNA, 84 bp, Artificial Sequence
  Synthetic oligonucleotide
agtagtcggcatggtgctgagcaccctccgggaaccgtccctacctgaaaggctaccgagccgcctgtacatttgctctgcctt

SEQ ID NO: 53 - DNA, 86 bp, Artificial Sequence
  Synthetic oligonucleotide
aattctagctccaaaatctgggctcctgaccacaatgttagaggtgccttgcattagtgtggagacctgtacatttgctctgcctt

SEQ ID NO: 54 - DNA, 84 bp, Artificial Sequence
  Synthetic oligonucleotide
gaaccgtcaccaggtcctttattgcctcttccaacaatagaaattatccatcgcaagatcgtgcctgtacatttgctctgcctt

SEQ ID NO: 55 - DNA, 86 bp, Artificial Sequence
  Synthetic oligonucleotide
agtcctaacctaggttacagcccatcacagctggagcaggtgatgcttagaacctgatacaccatcctgtacatttgctctgcctt

SEQ ID NO: 56 - DNA, 82 bp, Artificial Sequence
  Synthetic oligonucleotide
gtcaggcttaagaggcagggccacctaaacgtctactgagaacacagggtattggcgagcccctgtacatttgctctgcctt

SEQ ID NO: 57 - DNA, 84 bp, Artificial Sequence
  Synthetic oligonucleotide
ttcagatatgactagggaatgtttagaaagtacacgccacatgcttcatgactaggcgaggtccctgtacatttgctctgcctt

SEQ ID NO: 58 - DNA, 85 bp, Artificial Sequence
  Synthetic oligonucleotide
ctccaggtatagatgcaagtaggctggtagatttaatgaggagacaaactcgttcagttcagcccctgtacatttgctctgcctt

SEQ ID NO: 59 - DNA, 85 bp, Artificial Sequence
  Synthetic oligonucleotide
ctgggtgtaaagtttctgtgcaaacctttgctacagtgcgtgccgagtcaaagacatccgcgagcctgtacatttgctctgcctt

SEQ ID NO: 60 - DNA, 82 bp, Artificial Sequence
  Synthetic oligonucleotide
gacctgtagtcacaagtgtagagagtttgagcttcgacttagaaaggcacaccacgatactcctgtacatttgctctgcctt

SEQ ID NO: 61 - DNA, 83 bp, Artificial Sequence
  Synthetic oligonucleotide
ttcactggcgatcaacagtaaccaataaaattcactcatgaatcaacagtccttccggtaggcctgtacatttgctctgcctt

SEQ ID NO: 62 - DNA, 84 bp, Artificial Sequence
  Synthetic oligonucleotide
tctctgtagtcaatttgatttttatcaagttgcactaaatattttaagaccctatcactagagcctgtacatttgctctgcctt

SEQ ID NO: 63 - DNA, 87 bp, Artificial Sequence
  Synthetic oligonucleotide
cagcctgtgttcaggatctcacaaagtctctcatgaaaatagttgtgggcacctatacgatgaatccctgtacatttgctctgcctt

SEQ ID NO: 64 - DNA, 83 bp, Artificial Sequence
  Synthetic oligonucleotide
ttgttctcatctctcagaagcccttctgtggcccaaacattattgaccatcgccagacttgccctgtacatttgctctgcctt

SEQ ID NO: 65 - DNA, 83 bp, Artificial Sequence
  Synthetic oligonucleotide
gaagcctaggtatgtaaattacaggcttgcagaagtaaatgcaggtggctcgccatgatgtccctgtacatttgctctgcctt

SEQ ID NO: 66 - DNA, 85 bp, Artificial Sequence
  Synthetic oligonucleotide
ggacgagccccagaaaagtggaagaagactaatgatgccaccttggccccgatgatagttccagcctgtacatttgctctgcctt

SEQ ID NO: 67 - DNA, 82 bp, Artificial Sequence
  Synthetic oligonucleotide
tgccctcgtccctactggtaagaggcataaggtggggaagggcctaagtgctcaccagtctcctgtacatttgctctgcctt

SEQ ID NO: 68 - DNA, 89 bp, Artificial Sequence
  Synthetic oligonucleotide
cttaaaactaaaacaggaaaaaaaaatcaaaaccgtaacaaatcagtaatcacgatgttacgtggctgcctgtacatttgctctgcctt

SEQ ID NO: 69 - DNA, 84 bp, Artificial Sequence
  Synthetic oligonucleotide
aaatcagtaaaatgtttacaagcaatatcttttacgatcttaaaactaagacgaccatcctcacctgtacatttgctctgcctt

SEQ ID NO: 70 - DNA, 85 bp, Artificial Sequence
  Synthetic oligonucleotide
ctacataacagaattcagtatgcagtcatgatacatactctcagcaaagttatcgagccattggcctgtacatttgctctgcctt

SEQ ID NO: 71 - DNA, 82 bp, Artificial Sequence
  Synthetic oligonucleotide
tcctctcagtctctgagctctgtagaggagcctcaggggcagatgcaaccgaagaaggtatcctgtacatttgctctgcctt

SEQ ID NO: 72 - DNA, 85 bp, Artificial Sequence
  Synthetic oligonucleotide
acacaaaactaaaagcacttttaatatttcttcaaaacttctttcaattccaacgagagagcgacctgtacatttgctctgcctt

SEQ ID NO: 73 - DNA, 83 bp, Artificial Sequence
  Synthetic oligonucleotide
agccactccactcctaggtatctgcccgagagacatgaaagcacaaggaccatcggtcgtcgcctgtacatttgctctgcctt

SEQ ID NO: 74 - DNA, 81 bp, Artificial Sequence
  Synthetic oligonucleotide
tgatccccaacagagagaggtacccgggatcttctgacgtggttcatgtgcgcatcgtttcctgtacatttgctctgcctt

SEQ ID NO: 75 - DNA, 85 bp, Artificial Sequence
  Synthetic oligonucleotide
tccgaattctccaactttcctcccagcacgggtctgcccttgggactcgggcttcagtcatgttcctgtacatttgctctgcctt

SEQ ID NO: 76 - DNA, 84 bp, Artificial Sequence
  Synthetic oligonucleotide
tttcctttctttcttccaaactcctcttaatattggtattttgacctcctagaacgccagggacctgtacatttgctctgcctt

SEQ ID NO: 77 - DNA, 85 bp, Artificial Sequence
  Synthetic oligonucleotide
acatagaaggtgttcagtaaatatttcctgacagtaggagttgatgaatgcaggtatggagcagcctgtacatttgctctgcctt

SEQ ID NO: 78 - DNA, 84 bp, Artificial Sequence
  Synthetic oligonucleotide
tcatggccggtggccggttctcaccccttttgctcctaacagacacgcagagcctaacactgccctgtacatttgctctgcctt

SEQ ID NO: 79 - DNA, 85 bp, Artificial Sequence
  Synthetic oligonucleotide
ggagatactgacaattgcaagttgggctgatatgcatgaaaacagaaaatatcgagaacccgcacctgtacatttgctctgcctt

SEQ ID NO: 80 - DNA, 85 bp, Artificial Sequence
  Synthetic oligonucleotide
taacaaagactagcttatactacccacactttcctgtcatttttcttttcggtagttccaattgcctgtacatttgctctgcctt

SEQ ID NO: 81 - DNA, 83 bp, Artificial Sequence
  Synthetic oligonucleotide
aacattttgttttataatctgcgtctgataataccgatatacaaactctgcaatggagttggcctgtacatttgctctgcctt

SEQ ID NO: 82 - DNA, 85 bp, Artificial Sequence
  Synthetic oligonucleotide
agatggtgaagtaaagatgaataacatgaagcacatttgaatgctattggccttgattctcgttcctgtacatttgctctgcctt

SEQ ID NO: 83 - DNA, 88 bp, Artificial Sequence
  Synthetic oligonucleotide
tagtgatatttcaatacatataatgtatagtgatcagtgtaattagcataatgtacgattattagaccctgtacatttgctctgcctt

SEQ ID NO: 84 - DNA, 87 bp, Artificial Sequence
  Synthetic oligonucleotide
ctttctctaggtgccgtacatgttagtgggagctccttatttcctggattggagaggtaggtcttgcctgtacatttgctctgcctt

SEQ ID NO: 85 - DNA, 86 bp, Artificial Sequence
  Synthetic oligonucleotide
aactctcagtttgggccactgctctccagttgcctggagttttaagtcttacgacctgtgataggcctgtacatttgctctgcctt

SEQ ID NO: 86 - DNA, 83 bp, Artificial Sequence
  Synthetic oligonucleotide
atggccaagccttggctgttgagtaggcactgcccagttgtgctgtatggttgagccatggtcctgtacatttgctctgcctt

SEQ ID NO: 87 - DNA, 85 bp, Artificial Sequence
  Synthetic oligonucleotide
tcccgggctccgcatgctgcagcacatggttcaggatgcgctgctccttgattccttactcctacctgtacatttgctctgcctt

SEQ ID NO: 88 - DNA, 78 bp, Artificial Sequence
  Synthetic oligonucleotide
gaaggaccccagctccaccaaccaacaaaggcacagtccgtcccacccaccttattgcctgtacatttgctctgcctt

SEQ ID NO: 89 - DNA, 87 bp, Artificial Sequence
  Synthetic oligonucleotide
gaaatagaccctcgacagacccaaaggggcccacgtgatgcggtggtgacgttagagaagtaaggacctgtacatttgctctgcctt

SEQ ID NO: 90 - DNA, 83 bp, Artificial Sequence
  Synthetic oligonucleotide
cccagattttgctattccatacagttgactggacatgaactcattttggtggcgtacggaggcctgtacatttgctctgcctt

SEQ ID NO: 91 - DNA, 83 bp, Artificial Sequence
  Synthetic oligonucleotide
attctgaaaggaatgaaaatggggtttaaatgtccttaaggtccagagggcatcttccagttcctgtacatttgctctgcctt

SEQ ID NO: 92 - DNA, 88 bp, Artificial Sequence
  Synthetic oligonucleotide
attctgaagatttatcgtgaaaaaaaaagaatgtacaatcttttattaatatcgaatccaacatactcctgtacatttgctctgcctt

SEQ ID NO: 93 - DNA, 82 bp, Artificial Sequence
  Synthetic oligonucleotide
caacctgcccctccctgacccggggccccctttcctccagggcccagtctcgcctgctgaacctgtacatttgctctgcctt

SEQ ID NO: 94 - DNA, 85 bp, Artificial Sequence
  Synthetic oligonucleotide
gttgacttcttttaaaatatgatcttcacaattaccatccaatcaaattgtgagaagctgcgttcctgtacatttgctctgcctt

SEQ ID NO: 95 - DNA, 86 bp, Artificial Sequence
  Synthetic oligonucleotide
atagctttaccattttaccttgctcaatacgcaccccagacaaagagacacgtgatatctcccaacctgtacatttgctctgcctt

SEQ ID NO: 96 - DNA, 82 bp, Artificial Sequence
  Synthetic oligonucleotide
tttgttagcagggttggatctaaccagtgatgtgcggcatgtcagtgtgagcttacatttccctgtacatttgctctgcctt

SEQ ID NO: 97 - DNA, 89 bp, Artificial Sequence
  Synthetic oligonucleotide
cctcgttacctgcttctcatctgtgatgctccccagatctctgcaacatttgccgaggtataagcggacctgtacatttgctctgcctt

SEQ ID NO: 98 - DNA, 84 bp, Artificial Sequence
  Synthetic oligonucleotide
gcctggggccgggcggcaggggcgcgcagggtggcggcccagaggcctctattagtagtagaccctgtacatttgctctgcctt

SEQ ID NO: 99 - DNA, 79 bp, Artificial Sequence
  Synthetic oligonucleotide
gagagagggtgctaggctgctggcccagcaaggcctcaagggaagcagggacattagtcctgtacatttgctctgcctt

SEQ ID NO: 100 - DNA, 86 bp, Artificial Sequence
  Synthetic oligonucleotide
ggggttgggggggtggtgttgaggtatgtgtaagtctattgctcatgatagccaccaattatcgacctgtacatttgctctgcctt

SEQ ID NO: 101 - DNA, 87 bp, Artificial Sequence
  Synthetic oligonucleotide
aggcgggaacataaactaacaaaaaagtatgtcacagcacagtgacatgtttctggcagtacatctcctgtacatttgctctgcctt

SEQ ID NO: 102 - DNA, 80 bp, Artificial Sequence
  Synthetic oligonucleotide
tgatgggagcacaccccccaatgaccctgcccccacacctgcaaaagcccttatagactcctgtacatttgctctgcctt

SEQ ID NO: 103 - DNA, 85 bp, Artificial Sequence
  Synthetic oligonucleotide
ttaaagcacattaaagctcattagccactatgtcgaggccttatctaatcaagtccatcctgaccctgtacatttgctctgcctt

SEQ ID NO: 104 - DNA, 85 bp, Artificial Sequence
  Synthetic oligonucleotide
tagtatatcatataaaaataaagacatcacccaacataaaaacatcacccttcgaggtgttggccctgtacatttgctctgcctt

SEQ ID NO: 105 - DNA, 84 bp, Artificial Sequence
  Synthetic oligonucleotide
atgttgaactcttttgtcaaaagccccttgttggaaaagtctagagacttcgcatgacatcttcctgtacatttgctctgcctt

SEQ ID NO: 106 - DNA, 87 bp, Artificial Sequence
  Synthetic oligonucleotide
aaaccgtatgtgatctagcaatggaggagagggtcagaagtccccagtaccttgcatagagaaaatcctgtacatttgctctgcctt

SEQ ID NO: 107 - DNA, 86 bp, Artificial Sequence
  Synthetic oligonucleotide
tcaaatttcccgtgatcattactgcccatttcccaaaatatctcattgcacatcttaagccacggcctgtacatttgctctgcctt

SEQ ID NO: 108 - DNA, 84 bp, Artificial Sequence
  Synthetic oligonucleotide
ggtcatgataagtaagcagtgaaacaaagtagacatatgaatcagtaaaacgttacctctagacctgtacatttgctctgcctt

SEQ ID NO: 109 - DNA, 87 bp, Artificial Sequence
  Synthetic oligonucleotide
cttcactcgcagtaaatgtctatttctcctgtttcattaaaaaggttgagttaactacacacaaggcctgtacatttgctctgcctt

SEQ ID NO: 110 - DNA, 83 bp, Artificial Sequence
  Synthetic oligonucleotide
ctctgcccacggtatacctgggagagtgcaggtccttcagaaaggtgagttcggtcatccttcctgtacatttgctctgcctt

SEQ ID NO: 111 - DNA, 88 bp, Artificial Sequence
  Synthetic oligonucleotide
tgatcatatggtttttgtttttaattctgtttatatggtgaatcacacttacagtccgataacaccgcctgtacatttgctctgcctt

SEQ ID NO: 112 - DNA, 89 bp, Artificial Sequence
  Synthetic oligonucleotide
agatagatgacttagaggcccttgggtgtaacagagagtcagtcttcccaaatagaatcatctcatgacctgtacatttgctctgcctt

SEQ ID NO: 113 - DNA, 94 bp, Artificial Sequence
  Synthetic oligonucleotide
aatcttcataaaacctcagtgaatactcttttttcctgttaaaaaaatcttaataataacgaattataaccggcctgtacatttgctctgcctt

SEQ ID NO: 114 - DNA, 85 bp, Artificial Sequence
  Synthetic oligonucleotide
cttgcttatgaacactaatttcatatataaaacaaaaaatttattgtgataaccactgcatatgcctgtacatttgctctgcctt

SEQ ID NO: 115 - DNA, 46 bp, Artificial Sequence
  Synthetic oligonucleotide
tgactgagagagctccttggaatcaccaacaaacatgccatctcct

SEQ ID NO: 116 - DNA, 53 bp, Artificial Sequence
  Synthetic oligonucleotide
aagtagttaatctggaagtgtgaccaggacatggacgaggacgatggcagaga

SEQ ID NO: 117 - DNA, 42 bp, Artificial Sequence
  Synthetic oligonucleotide
cggtgctcttgtatatagacggtaaaataaacaccaagaggt

SEQ ID NO: 118 - DNA, 46 bp, Artificial Sequence
  Synthetic oligonucleotide
gcacatactcatcaattcatacagaacaatcccaaatgcatataca

SEQ ID NO: 119 - DNA, 47 bp, Artificial Sequence
  Synthetic oligonucleotide
tgatgtatcaaaaatcatcattttcatctctgtatatggatgtctgt

SEQ ID NO: 120 - DNA, 49 bp, Artificial Sequence
  Synthetic oligonucleotide
tcagttgtagagaagctttagtagattatgctggtattagggtggaagg

SEQ ID NO: 121 - DNA, 47 bp, Artificial Sequence
  Synthetic oligonucleotide
tctgcagtaaaagtgacccaggtgagttttgtttcacattataacca

SEQ ID NO: 122 - DNA, 46 bp, Artificial Sequence
  Synthetic oligonucleotide
tctcagtgcgcaggagagccaggatggcagcgggagccgcccggcg

SEQ ID NO: 123 - DNA, 51 bp, Artificial Sequence
  Synthetic oligonucleotide
atatatatcggaaaacaactcaccctgaaggccaaattatgtcatagtgca

SEQ ID NO: 124 - DNA, 48 bp, Artificial Sequence
  Synthetic oligonucleotide
agactattgccaacacaacaacactaaggtcagtccctgcagatcaca

SEQ ID NO: 125 - DNA, 51 bp, Artificial Sequence
  Synthetic oligonucleotide
tgatattaacataaagacaagggcaacaagccctcattccgtggagtgtct

SEQ ID NO: 126 - DNA, 44 bp, Artificial Sequence
  Synthetic oligonucleotide
gctcgtcacagtctgaatgagttatgttgtggttcccccaacat

SEQ ID NO: 127 - DNA, 43 bp, Artificial Sequence
  Synthetic oligonucleotide
cacatgcggtgtagctggtctcttggtccttgggatgtgcatg

SEQ ID NO: 128 - DNA, 49 bp, Artificial Sequence
  Synthetic oligonucleotide
tggcagctttggccagctgcggagccattcagccggacgtcctgcactt

SEQ ID NO: 129 - DNA, 50 bp, Artificial Sequence
  Synthetic oligonucleotide
tagcaatcaagcaccttggctctgtctgaaggccagtgacgcagtgacag

SEQ ID NO: 130 - DNA, 48 bp, Artificial Sequence
  Synthetic oligonucleotide
gaagcaccagtagagtggtggatccagacaccaacagatgccagagaa

SEQ ID NO: 131 - DNA, 49 bp, Artificial Sequence
  Synthetic oligonucleotide
gtgagagattaaacagtcgtcatctggaaagaaatatcaacatgaggca

SEQ ID NO: 132 - DNA, 49 bp, Artificial Sequence
  Synthetic oligonucleotide
atagttaggtatcagctagacggcacgtatccaccgcctgtccatgatg

SEQ ID NO: 133 - DNA, 49 bp, Artificial Sequence
  Synthetic oligonucleotide
aggtatgaaccgtcgattcccagatcttaaccgatttgccaagaagttt

SEQ ID NO: 134 - DNA, 45 bp, Artificial Sequence
  Synthetic oligonucleotide
cggtatcaatcttgctttctgatataatttgttctgtaggcccca

SEQ ID NO: 135 - DNA, 47 bp, Artificial Sequence
  Synthetic oligonucleotide
gttctcccaccacgccctcgtcactggatgcggggcccgacactgtg

SEQ ID NO: 136 - DNA, 47 bp, Artificial Sequence
  Synthetic oligonucleotide
ttcgtcgtcaacatcgagatggcctatgcgcggcaagtgagcgtgat

SEQ ID NO: 137 - DNA, 52 bp, Artificial Sequence
  Synthetic oligonucleotide
ttgacgttaatatgatgaagagtttattgtttttgtaggagatagacagctt

SEQ ID NO: 138 - DNA, 49 bp, Artificial Sequence
  Synthetic oligonucleotide
tagatccattctgggactttccgggaacttatgtagaatatattggaag

SEQ ID NO: 139 - DNA, 41 bp, Artificial Sequence
  Synthetic oligonucleotide
atcggtgatacgtggttgcttaatgactccctttctttttc

SEQ ID NO: 140 - DNA, 47 bp, Artificial Sequence
  Synthetic oligonucleotide
acctctggtgttttcacagccacggttttcacaactctgcaacagaa

SEQ ID NO: 141 - DNA, 51 bp, Artificial Sequence
  Synthetic oligonucleotide
tcttcgagtgctcactccaggagggcaacggggcttctgatgagggcattt

SEQ ID NO: 142 - DNA, 49 bp, Artificial Sequence
  Synthetic oligonucleotide
ctgtatcgacttcctcttactctctgcctgcaggatgtgcggcgtgtgc

SEQ ID NO: 143 - DNA, 46 bp, Artificial Sequence
  Synthetic oligonucleotide
acttggcttgaaaacaataaatctaccccctgttgttagtgaggac

SEQ ID NO: 144 - DNA, 46 bp, Artificial Sequence
  Synthetic oligonucleotide
aagatttgcctgggccccgacgacatggcatccctgacctgaacca

SEQ ID NO: 145 - DNA, 50 bp, Artificial Sequence
  Synthetic oligonucleotide
aataacttaactgtgcctacaccatgtgtggctccatgctccgcacagtg

SEQ ID NO: 146 - DNA, 46 bp, Artificial Sequence
  Synthetic oligonucleotide
accggaccgttctcgatgatgtgcggcagagggaaggccctcacac

SEQ ID NO: 147 - DNA, 48 bp, Artificial Sequence
  Synthetic oligonucleotide
cacactaatatgctttctatccccagattgctcctgggaagataacag

SEQ ID NO: 148 - DNA, 51 bp, Artificial Sequence
  Synthetic oligonucleotide
actgccagaagtttatttcataggtagtaacattttccaggaagcatttaa

SEQ ID NO: 149 - DNA, 48 bp, Artificial Sequence
  Synthetic oligonucleotide
tgactggatcctaacgaggcttacgtggaggaaggaatggcactgcac

SEQ ID NO: 150 - DNA, 47 bp, Artificial Sequence
  Synthetic oligonucleotide
aaggctcgctcacagcccaaacttcaacaccaaaaatacttttccag

SEQ ID NO: 151 - DNA, 45 bp, Artificial Sequence
  Synthetic oligonucleotide
tccaccgccactggctcactctcgaattctgtcttgagcgaggac

SEQ ID NO: 152 - DNA, 45 bp, Artificial Sequence
  Synthetic oligonucleotide
tcactggactctcctcagctcaaaacccttcagtggcactccgtt

SEQ ID NO: 153 - DNA, 48 bp, Artificial Sequence
  Synthetic oligonucleotide
gattatgttgttctctaaactgacaactcacgcctgcccacgtcgggg

SEQ ID NO: 154 - DNA, 47 bp, Artificial Sequence
  Synthetic oligonucleotide
ggcctcgttgcacatggcaagcccggtcttccctgggagatcccaac

SEQ ID NO: 155 - DNA, 51 bp, Artificial Sequence
  Synthetic oligonucleotide
gctgattacatattaagagacaaaaatgttatactgcatatagaaaacaaa

SEQ ID NO: 156 - DNA, 46 bp, Artificial Sequence
  Synthetic oligonucleotide
ccggcattagagctcttctgtctcggttgtggtcccacagaacatt

SEQ ID NO: 157 - DNA, 48 bp, Artificial Sequence
  Synthetic oligonucleotide
tccaagagaaaagggtgaatgtttatagttcctgaagcccctgtgggc

SEQ ID NO: 158 - DNA, 41 bp, Artificial Sequence
  Synthetic oligonucleotide
tgcgttccgtatgtgtttctgagtgcagatagttgttctag

SEQ ID NO: 159 - DNA, 49 bp, Artificial Sequence
  Synthetic oligonucleotide
tgagtataggttcccagttactacttccaggattacaaacagaattgct

SEQ ID NO: 160 - DNA, 44 bp, Artificial Sequence
  Synthetic oligonucleotide
tcgaatggtacaaaaaacaatagagcttaaacaaaaatgaaggg

SEQ ID NO: 161 - DNA, 49 bp, Artificial Sequence
  Synthetic oligonucleotide
ccgccaatggtaaggggaccatcttagtaatggcacagaggaaacatgt

SEQ ID NO: 162 - DNA, 49 bp, Artificial Sequence
  Synthetic oligonucleotide
tctgaagcctagctcacgggacctcatggatcctggctccagtgagaca

SEQ ID NO: 163 - DNA, 45 bp, Artificial Sequence
  Synthetic oligonucleotide
tcactccgaaaaatcactccatgttattctaagaaactgtaagtc

SEQ ID NO: 164 - DNA, 46 bp, Artificial Sequence
  Synthetic oligonucleotide
attgcctgagtctcaactgatgtatccatttcattgtcgacagcca

SEQ ID NO: 165 - DNA, 42 bp, Artificial Sequence
  Synthetic oligonucleotide
agagccgccatttcagagtgagatggtgaggtgatcatgggc

SEQ ID NO: 166 - DNA, 49 bp, Artificial Sequence
  Synthetic oligonucleotide
tcggtagcctttcaggtagggacggttcccggagggtgctcagcaccat

SEQ ID NO: 167 - DNA, 48 bp, Artificial Sequence
  Synthetic oligonucleotide
cacactaatgcaaggcacctctaacattgtggtcaggagcccagattt

SEQ ID NO: 168 - DNA, 49 bp, Artificial Sequence
  Synthetic oligonucleotide
atcttgcgatggataatttctattgttggaagaggcaataaaggacctg

SEQ ID NO: 169 - DNA, 48 bp, Artificial Sequence
  Synthetic oligonucleotide
tgtatcaggttctaagcatcacctgctccagctgtgatgggctgtaac

SEQ ID NO: 170 - DNA, 45 bp, Artificial Sequence
  Synthetic oligonucleotide
cgccaataccctgtgttctcagtagacgtttaggtggccctgcct

SEQ ID NO: 171 - DNA, 44 bp, Artificial Sequence
  Synthetic oligonucleotide
tcgcctagtcatgaagcatgtggcgtgtactttctaaacattcc

SEQ ID NO: 172 - DNA, 47 bp, Artificial Sequence
  Synthetic oligonucleotide
gaactgaacgagtttgtctcctcattaaatctaccagcctacttgca

SEQ ID NO: 173 - DNA, 48 bp, Artificial Sequence
  Synthetic oligonucleotide
cggatgtctttgactcggcacgcactgtagcaaaggtttgcacagaaa

SEQ ID NO: 174 - DNA, 45 bp, Artificial Sequence
  Synthetic oligonucleotide
tcgtggtgtgcctttctaagtcgaagctcaaactctctacacttg

SEQ ID NO: 175 - DNA, 48 bp, Artificial Sequence
  Synthetic oligonucleotide
ccggaaggactgttgattcatgagtgaattttattggttactgttgat

SEQ ID NO: 176 - DNA, 46 bp, Artificial Sequence
  Synthetic oligonucleotide
agtgatagggtcttaaaatatttagtgcaacttgataaaaatcaaa

SEQ ID NO: 177 - DNA, 51 bp, Artificial Sequence
  Synthetic oligonucleotide
catcgtataggtgcccacaactattttcatgagagactttgtgagatcctg

SEQ ID NO: 178 - DNA, 45 bp, Artificial Sequence
  Synthetic oligonucleotide
gtctggcgatggtcaataatgtttgggccacagaagggcttctga

SEQ ID NO: 179 - DNA, 45 bp, Artificial Sequence
  Synthetic oligonucleotide
tcatggcgagccacctgcatttacttctgcaagcctgtaatttac

SEQ ID NO: 180 - DNA, 51 bp, Artificial Sequence
  Synthetic oligonucleotide
aactatcatcggggccaaggtggcatcattagtcttcttccacttttctgg

SEQ ID NO: 181 - DNA, 48 bp, Artificial Sequence
  Synthetic oligonucleotide
tggtgagcacttaggcccttccccaccttatgcctcttaccagtaggg

SEQ ID NO: 182 - DNA, 48 bp, Artificial Sequence
  Synthetic oligonucleotide
cacgtaacatcgtgattactgatttgttacggttttgatttttttttc

SEQ ID NO: 183 - DNA, 44 bp, Artificial Sequence
  Synthetic oligonucleotide
gatggtcgtcttagttttaagatcgtaaaagatattgcttgtaa

SEQ ID NO: 184 - DNA, 45 bp, Artificial Sequence
  Synthetic oligonucleotide
tggctcgataactttgctgagagtatgtatcatgactgcatactg

SEQ ID NO: 185 - DNA, 45 bp, Artificial Sequence
  Synthetic oligonucleotide
cttcttcggttgcatctgcccctgaggctcctctacagagctcag

SEQ ID NO: 186 - DNA, 46 bp, Artificial Sequence
  Synthetic oligonucleotide
tctctcgttggaattgaaagaagttttgaagaaatattaaaagtgc

SEQ ID NO: 187 - DNA, 48 bp, Artificial Sequence
  Synthetic oligonucleotide
gaccgatggtccttgtgctttcatgtctctcgggcagatacctaggag

SEQ ID NO: 188 - DNA, 45 bp, Artificial Sequence
  Synthetic oligonucleotide
gatgcgcacatgaaccacgtcagaagatcccgggtacctctctct

SEQ ID NO: 189 - DNA, 48 bp, Artificial Sequence
  Synthetic oligonucleotide
tgactgaagcccgagtcccaagggcagacccgtgctgggaggaaagtt

SEQ ID NO: 190 - DNA, 45 bp, Artificial Sequence
  Synthetic oligonucleotide
tggcgttctaggaggtcaaaataccaatattaagaggagtttgga

SEQ ID NO: 191 - DNA, 47 bp, Artificial Sequence
  Synthetic oligonucleotide
tccatacctgcattcatcaactcctactgtcaggaaatatttactga

SEQ ID NO: 192 - DNA, 50 bp, Artificial Sequence
  Synthetic oligonucleotide
tgttaggctctgcgtgtctgttaggagcaaaaggggtgagaaccggccac

SEQ ID NO: 193 - DNA, 47 bp, Artificial Sequence
  Synthetic oligonucleotide
ggttctcgatattttctgttttcatgcatatcagcccaacttgcaat

SEQ ID NO: 194 - DNA, 46 bp, Artificial Sequence
  Synthetic oligonucleotide
tggaactaccgaaaagaaaaatgacaggaaagtgtgggtagtataa

SEQ ID NO: 195 - DNA, 41 bp, Artificial Sequence
  Synthetic oligonucleotide
ctccattgcagagtttgtatatcggtattatcagacgcaga

SEQ ID NO: 196 - DNA, 47 bp, Artificial Sequence
  Synthetic oligonucleotide
agaatcaaggccaatagcattcaaatgtgcttcatgttattcatctt

SEQ ID NO: 197 - DNA, 47 bp, Artificial Sequence
  Synthetic oligonucleotide
aataatcgtacattatgctaattacactgatcactatacattatatg

SEQ ID NO: 198 - DNA, 49 bp, Artificial Sequence
  Synthetic oligonucleotide
acctacctctccaatccaggaaataaggagctcccactaacatgtacgg

SEQ ID NO: 199 - DNA, 48 bp, Artificial Sequence
  Synthetic oligonucleotide
tcacaggtcgtaagacttaaaactccaggcaactggagagcagtggcc

SEQ ID NO: 200 - DNA, 48 bp, Artificial Sequence
  Synthetic oligonucleotide
tggctcaaccatacagcacaactgggcagtgcctactcaacagccaag

SEQ ID NO: 201 - DNA, 52 bp, Artificial Sequence
  Synthetic oligonucleotide
agtaaggaatcaaggagcagcgcatcctgaaccatgtgctgcagcatgcgga

SEQ ID NO: 202 - DNA, 45 bp, Artificial Sequence
  Synthetic oligonucleotide
ataaggtgggtgggacggactgtgcctttgttggttggtggagct

SEQ ID NO: 203 - DNA, 49 bp, Artificial Sequence
  Synthetic oligonucleotide
tacttctctaacgtcaccaccgcatcacgtgggcccctttgggtctgtc

SEQ ID NO: 204 - DNA, 45 bp, Artificial Sequence
  Synthetic oligonucleotide
gtacgccaccaaaatgagttcatgtccagtcaactgtatggaata

SEQ ID NO: 205 - DNA, 43 bp, Artificial Sequence
  Synthetic oligonucleotide
gaagatgccctctggaccttaaggacatttaaaccccattttc

SEQ ID NO: 206 - DNA, 47 bp, Artificial Sequence
  Synthetic oligonucleotide
tgttggattcgatattaataaaagattgtacattctttttttttcac

SEQ ID NO: 207 - DNA, 46 bp, Artificial Sequence
  Synthetic oligonucleotide
caggcgagactgggccctggaggaaagggggccccgggtcagggag

SEQ ID NO: 208 - DNA, 45 bp, Artificial Sequence
  Synthetic oligonucleotide
cagcttctcacaatttgattggatggtaattgtgaagatcatatt

SEQ ID NO: 209 - DNA, 48 bp, Artificial Sequence
  Synthetic oligonucleotide
ggagatatcacgtgtctctttgtctggggtgcgtattgagcaaggtaa

SEQ ID NO: 210 - DNA, 45 bp, Artificial Sequence
  Synthetic oligonucleotide
tgtaagctcacactgacatgccgcacatcactggttagatccaac

SEQ ID NO: 211 - DNA, 53 bp, Artificial Sequence
  Synthetic oligonucleotide
cttatacctcggcaaatgttgcagagatctggggagcatcacagatgagaagc

SEQ ID NO: 212 - DNA, 50 bp, Artificial Sequence
  Synthetic oligonucleotide
ctactaatagaggcctctgggccgccaccctgcgcgcccctgccgcccgg

SEQ ID NO: 213 - DNA, 41 bp, Artificial Sequence
  Synthetic oligonucleotide
gtccctgcttcccttgaggccttgctgggccagcagcctag

SEQ ID NO: 214 - DNA, 52 bp, Artificial Sequence
  Synthetic oligonucleotide
taattggtggctatcatgagcaatagacttacacatacctcaacaccacccc

SEQ ID NO: 215 - DNA, 53 bp, Artificial Sequence
  Synthetic oligonucleotide
gtactgccagaaacatgtcactgtgctgtgacatacttttttgttagtttatg

SEQ ID NO: 216 - DNA, 45 bp, Artificial Sequence
  Synthetic oligonucleotide
tataagggcttttgcaggtgtgggggcagggtcattggggggtgt

SEQ ID NO: 217 - DNA, 45 bp, Artificial Sequence
  Synthetic oligonucleotide
ggatggacttgattagataaggcctcgacatagtggctaatgagc

SEQ ID NO: 218 - DNA, 39 bp, Artificial Sequence
  Synthetic oligonucleotide
cacctcgaagggtgatgtttttatgttgggtgatgtctt

SEQ ID NO: 219 - DNA, 44 bp, Artificial Sequence
  Synthetic oligonucleotide
gtcatgcgaagtctctagacttttccaacaaggggcttttgaca

SEQ ID NO: 220 - DNA, 49 bp, Artificial Sequence
  Synthetic oligonucleotide
ctctatgcaaggtactggggacttctgaccctctcctccattgctagat

SEQ ID NO: 221 - DNA, 48 bp, Artificial Sequence
  Synthetic oligonucleotide
gcttaagatgtgcaatgagatattttgggaaatgggcagtaatgatca

SEQ ID NO: 222 - DNA, 44 bp, Artificial Sequence
  Synthetic oligonucleotide
aggtaacgttttactgattcatatgtctactttgtttcactgct

SEQ ID NO: 223 - DNA, 51 bp, Artificial Sequence
  Synthetic oligonucleotide
gtgtgtagttaactcaacctttttaatgaaacaggagaaatagacatttac

SEQ ID NO: 224 - DNA, 46 bp, Artificial Sequence
  Synthetic oligonucleotide
gaccgaactcacctttctgaaggacctgcactctcccaggtatacc

SEQ ID NO: 225 - DNA, 48 bp, Artificial Sequence
  Synthetic oligonucleotide
ttatcggactgtaagtgtgattcaccatataaacagaattaaaaacaa

SEQ ID NO: 226 - DNA, 48 bp, Artificial Sequence
  Synthetic oligonucleotide
agatgattctatttgggaagactgactctctgttacacccaagggcct

SEQ ID NO: 227 - DNA, 54 bp, Artificial Sequence
  Synthetic oligonucleotide
ttataattcgttattattaagatttttttaacaggaaaaaagagtattcactga

SEQ ID NO: 228 - DNA, 46 bp, Artificial Sequence
  Synthetic oligonucleotide
gcagtggttatcacaataaattttttgttttatatatgaaattagt

SEQ ID NO: 229 - DNA, 61 bp, Artificial Sequence
  Synthetic oligonucleotide
aaataatcaggagaaggagaaggcatgtttgttggtgattccaaggagctcgaagcatagg

SEQ ID NO: 230 - DNA, 64 bp, Artificial Sequence
  Synthetic oligonucleotide
ggagcagcgtctctgccatcgtcctcatccatgtcctggtcacacttccaagagtgtatttagt

SEQ ID NO: 231 - DNA, 62 bp, Artificial Sequence
  Synthetic oligonucleotide
aggtaaatatttaccacgtcttggtgtttattttaccgtctatatacaagatcatacattac

SEQ ID NO: 232 - DNA, 60 bp, Artificial Sequence
  Synthetic oligonucleotide
ctttcagtcagatgtatatgcatttggaattgttctgtatgaattgatgaaacggagaat

SEQ ID NO: 233 - DNA, 61 bp, Artificial Sequence
  Synthetic oligonucleotide
tatctgctaagaaacaggcatccatatacagagatgaaaatgatgattttgataacctata

SEQ ID NO: 234 - DNA, 65 bp, Artificial Sequence
  Synthetic oligonucleotide
atcttgccttgccttccaccgtaataccagcataatctactaaagcttctactaatacacacatc

SEQ ID NO: 235 - DNA, 60 bp, Artificial Sequence
  Synthetic oligonucleotide
tgtccagtgatatggttatcatgtgaaacaaaactcacctgggtcactttcgactagaag

SEQ ID NO: 236 - DNA, 62 bp, Artificial Sequence
  Synthetic oligonucleotide
attaaaggcgccgccgggcggctcccgctgacatcctggctctcctgcgcttcgcgcgaatg

SEQ ID NO: 237 - DNA, 60 bp, Artificial Sequence
  Synthetic oligonucleotide
gatttgcgttctgcactatgacatcatttggccttcagggtgagttgtttattgcagtat

SEQ ID NO: 238 - DNA, 58 bp, Artificial Sequence
  Synthetic oligonucleotide
agacttcaccttgtgatctgcagggactgaccttggtgttgttgtgttggccgaaacc

SEQ ID NO: 239 - DNA, 58 bp, Artificial Sequence
  Synthetic oligonucleotide
agagatctccaaagacactccacggaatgagggctcgttgcccttgtcttgcgtgatt

SEQ ID NO: 240 - DNA, 60 bp, Artificial Sequence
  Synthetic oligonucleotide
cctatacaattgagatggtgggggaaccacaacataactcattcagactgcggacaattc

SEQ ID NO: 241 - DNA, 59 bp, Artificial Sequence
  Synthetic oligonucleotide
tttttattgtatatgcatgcgcatcccaaggaccaagagaccagctacaccgcttaggt

SEQ ID NO: 242 - DNA, 61 bp, Artificial Sequence
  Synthetic oligonucleotide
gcaggcatcaaagtgcacgacgtccggctgaatggctccgcagctggccaatgtacttagt

SEQ ID NO: 243 - DNA, 59 bp, Artificial Sequence
  Synthetic oligonucleotide
tctgcattccctgtcaccgcgtcactggccttcagacagagccaaggtgcctgcttgtg

SEQ ID NO: 244 - DNA, 62 bp, Artificial Sequence
  Synthetic oligonucleotide
gcccaagtgtttctctggcatctgatggtgtctggatccaccactctactatgcttgaaatc

SEQ ID NO: 245 - DNA, 65 bp, Artificial Sequence
  Synthetic oligonucleotide
gttcagatttcactgcctcatgttgatgtttctttccagatgacgactgtcttaatacatggttc

SEQ ID NO: 246 - DNA, 61 bp, Artificial Sequence
  Synthetic oligonucleotide
tagacacattgtcatcatggacgggcggtggatacgtgccgtctagctgagtcttcataat

SEQ ID NO: 247 - DNA, 62 bp, Artificial Sequence
  Synthetic oligonucleotide
gcttgtctttgaaacttcttggcaagtcggttaagatctgggaatcgacggatattggaaaa

SEQ ID NO: 248 - DNA, 61 bp, Artificial Sequence
  Synthetic oligonucleotide
tacctcccatattggggcctacaaaacaaattatatcagaaagcaagattatgatcacaaa

SEQ ID NO: 249 - DNA, 59 bp, Artificial Sequence
  Synthetic oligonucleotide
tgccaatgaccacagtgtcgggcccggcatccagtgacgagggcgtggtgggctcgatt

SEQ ID NO: 250 - DNA, 60 bp, Artificial Sequence
  Synthetic oligonucleotide
atcccccttaaaatcacactcacttgccgcgcataggccatctcgatgttggctggacta

SEQ ID NO: 251 - DNA, 60 bp, Artificial Sequence
  Synthetic oligonucleotide
ggggaggtgaagctgtccatctcctacaaaaacaataaactcttcatcatattctcaact

SEQ ID NO: 252 - DNA, 63 bp, Artificial Sequence
  Synthetic oligonucleotide
cgagatttttttccttccaatatattctacgtaagttcccggaaagtccctatgatcgaaatc

SEQ ID NO: 253 - DNA, 58 bp, Artificial Sequence
  Synthetic oligonucleotide
cttttccttaaaaagaaaaagaaagggagtcattaagcaaccacttatcacacgtatg

SEQ ID NO: 254 - DNA, 60 bp, Artificial Sequence
  Synthetic oligonucleotide
tgtgtgttatgatttctcttgcagagttgtgaaaaccgtggctgtgaaaagagctctgta

SEQ ID NO: 255 - DNA, 61 bp, Artificial Sequence
  Synthetic oligonucleotide
ggccctgcaaatgccctcagcagaagccccgttgccctcctggagtgagcaagcaagagtc

SEQ ID NO: 256 - DNA, 60 bp, Artificial Sequence
  Synthetic oligonucleotide
gctactccaggcacacgtcgcacatcctgcaggcagagagtaagaggaagtcacgcaaac

SEQ ID NO: 257 - DNA, 55 bp, Artificial Sequence
  Synthetic oligonucleotide
gatctctcctgagtcctcactaacaacagggggttgatttattgttttcaagagc

SEQ ID NO: 258 - DNA, 54 bp, Artificial Sequence
  Synthetic oligonucleotide
ccactagccctggttcaggtcagggatgccatgttgtcggggcccaggcaaata

SEQ ID NO: 259 - DNA, 53 bp, Artificial Sequence
  Synthetic oligonucleotide
tggcagcctcactgtgcggagcatggagccacacgtggtgtaggcacagtggt

SEQ ID NO: 260 - DNA, 58 bp, Artificial Sequence
  Synthetic oligonucleotide
aagtaccccaaagtgtgagggccttccctctgccacacatcatcgagaacttatggct

SEQ ID NO: 261 - DNA, 55 bp, Artificial Sequence
  Synthetic oligonucleotide
gtgcttctgaaactgttatcttcccaggagcaatttggggatagaaagcatcagt

SEQ ID NO: 262 - DNA, 56 bp, Artificial Sequence
  Synthetic oligonucleotide
gggccagtctttaaatgcttcctggaaaatgttgctacctatgaaataaaccttgt

SEQ ID NO: 263 - DNA, 57 bp, Artificial Sequence
  Synthetic oligonucleotide
aaggaagcagcgtgcagtgccattccttcctccaggtaagcctcgttagggttaggt

SEQ ID NO: 264 - DNA, 58 bp, Artificial Sequence
  Synthetic oligonucleotide
attgggggtatattggaaaagtatttttggtgttgaagtttgggctgtgagccaagct

SEQ ID NO: 265 - DNA, 56 bp, Artificial Sequence
  Synthetic oligonucleotide
gggatgtttcttgtcctcgttcaagacagaattcgagagtgagccagtggggagtc

SEQ ID NO: 266 - DNA, 57 bp, Artificial Sequence
  Synthetic oligonucleotide
tttgacaccaataaaatggagtgccactgaagggttttgagctgaggagagaacgaa

SEQ ID NO: 267 - DNA, 57 bp, Artificial Sequence
  Synthetic oligonucleotide
accgtacctctgcccgacgtgggcaggcgtgagttgtcagtttagagaacattagag

SEQ ID NO: 268 - DNA, 55 bp, Artificial Sequence
  Synthetic oligonucleotide
ctgcttctagggttgggaactcccagggaagaccgggcttgccatgtgcacgaca

SEQ ID NO: 269 - DNA, 57 bp, Artificial Sequence
  Synthetic oligonucleotide
gtcattttgctgtttgttttctatatgcggtataacatttttgtctcttaggagaga

SEQ ID NO: 270 - DNA, 54 bp, Artificial Sequence
  Synthetic oligonucleotide
taggagacagagaatgttctgtgggaccacaaccaagacagaagagctctccgt

SEQ ID NO: 271 - DNA, 59 bp, Artificial Sequence
  Synthetic oligonucleotide
cctgtaacacacgcccacaggggcttcaggaactgtaaacattcaccctttatagtcag

SEQ ID NO: 272 - DNA, 57 bp, Artificial Sequence
  Synthetic oligonucleotide
tatttgataaattaaccctagaacaactatctgcgctcagaaacacataccatcctc

SEQ ID NO: 273 - DNA, 57 bp, Artificial Sequence
  Synthetic oligonucleotide
gagcatcctgaagcaattctgtttgtaatcctgggagtagtaactgggaatgagatt

SEQ ID NO: 274 - DNA, 57 bp, Artificial Sequence
  Synthetic oligonucleotide
tgaattatttttcttccctttcatttttgtttaagctctattgttttttgttatgga

SEQ ID NO: 275 - DNA, 58 bp, Artificial Sequence
  Synthetic oligonucleotide
agaacaatgtccacatgtttcctctgtgccattattaagatggtccccttgccatcac

SEQ ID NO: 276 - DNA, 55 bp, Artificial Sequence
  Synthetic oligonucleotide
catcccaccctgtctcactggagccaggatccataaggtcccgtgagctagcttg

SEQ ID NO: 277 - DNA, 56 bp, Artificial Sequence
  Synthetic oligonucleotide
tccatcctaaaggacttacggtttcttagaataacatggagtgatttttcagagta

SEQ ID NO: 278 - DNA, 56 bp, Artificial Sequence
  Synthetic oligonucleotide
gaaaacagtcaaaatggctgtcaacaatgaaatggatacatcagttgagagaccat

SEQ ID NO: 279 - DNA, 57 bp, Artificial Sequence
  Synthetic oligonucleotide
gaaagactaataattttgcccatgatcacctcactatctcactctgaaatggcacag

SEQ ID NO: 280 - DNA, 54 bp, Artificial Sequence
  Synthetic oligonucleotide
agtagtcggcatggtgctgagcatcctccgggaaccgtccctacctgaaaggca

SEQ ID NO: 281 - DNA, 58 bp, Artificial Sequence
  Synthetic oligonucleotide
aattctagctccaaaatctgggctcctgaccacagtgttagaggtgcctttacgattg

SEQ ID NO: 282 - DNA, 56 bp, Artificial Sequence
  Synthetic oligonucleotide
gaaccgtcaccaggtcctttattgcctcttccaataatagaaattatccatccgtg

SEQ ID NO: 283 - DNA, 59 bp, Artificial Sequence
  Synthetic oligonucleotide
agtcctaacctaggttacagcccatcacagctggggcaggtgatgcttagttatgtaat

SEQ ID NO: 284 - DNA, 51 bp, Artificial Sequence
  Synthetic oligonucleotide
gtcaggcttaagaggcagggccacctaaacgtctgctgagaacacagggtt

SEQ ID NO: 285 - DNA, 57 bp, Artificial Sequence
  Synthetic oligonucleotide
ttcagatatgactagggaatgtttagaaagtacaggccacatgcttcatgagtagta

SEQ ID NO: 286 - DNA, 55 bp, Artificial Sequence
  Synthetic oligonucleotide
ctccaggtatagatgcaagtaggctggtagatttgatgaggagacaaacttcgga

SEQ ID NO: 287 - DNA, 55 bp, Artificial Sequence
  Synthetic oligonucleotide
ctgggtgtaaagtttctgtgcaaacctttgctacggtgcgtgccgagtcacgcgt

SEQ ID NO: 288 - DNA, 59 bp, Artificial Sequence
  Synthetic oligonucleotide
gacctgtagtcacaagtgtagagagtttgagctttgacttagaaaggcacattagtcta

SEQ ID NO: 289 - DNA, 58 bp, Artificial Sequence
  Synthetic oligonucleotide
ttcactggcgatcaacagtaactaataaaattcactcatgaatcaacagtacatgcag

SEQ ID NO: 290 - DNA, 55 bp, Artificial Sequence
  Synthetic oligonucleotide
tctctgtagtcaatttgatttttatcaagttgcattaaatattttaagacggtag

SEQ ID NO: 291 - DNA, 58 bp, Artificial Sequence
  Synthetic oligonucleotide
cagcctgtgttcaggatctcacagagtctctcatgaaaatagttgtgggcataagcca

SEQ ID NO: 292 - DNA, 57 bp, Artificial Sequence
  Synthetic oligonucleotide
ttgttctcatctctcagatgcccttctgtggcccaaacattattgaccatatcctaa

SEQ ID NO: 293 - DNA, 57 bp, Artificial Sequence
  Synthetic oligonucleotide
gaagcctaggtatgtaaattataggcttgcagaagtaaatgcaggtggctttcgatt

SEQ ID NO: 294 - DNA, 59 bp, Artificial Sequence
  Synthetic oligonucleotide
ggacgagccccagaaaagtggaagaagactaatggtgccaccttggcccccatactctt

SEQ ID NO: 295 - DNA, 60 bp, Artificial Sequence
  Synthetic oligonucleotide
tgccctcggccctactggtaagaggcataaggtggggaagggcctaagtgaattattcat

SEQ ID NO: 296 - DNA, 58 bp, Artificial Sequence
  Synthetic oligonucleotide
cttaaaactaaaacaggaaaaaaaaatcaaaaccataacaaatcagtaattggatcca

SEQ ID NO: 297 - DNA, 56 bp, Artificial Sequence
  Synthetic oligonucleotide
aaatcagtaaaatgtttacaagcaatatcttttatgatcttaaaactaagagcgct

SEQ ID NO: 298 - DNA, 57 bp, Artificial Sequence
  Synthetic oligonucleotide
ctacataacagaattcagtatgcagtcatgatacgtactctcagcaaagttgagttc

SEQ ID NO: 299 - DNA, 55 bp, Artificial Sequence
  Synthetic oligonucleotide
tcctctcagtctctgagctctgtagaggagcctcgggggcagatgcaaccttaga

SEQ ID NO: 300 - DNA, 55 bp, Artificial Sequence
  Synthetic oligonucleotide
acacaaaactaaaagcacttttaatatttcttcagaacttctttcaattcggaga

SEQ ID NO: 301 - DNA, 55 bp, Artificial Sequence
  Synthetic oligonucleotide
agccactccactcctaggtatctgcccaagagacatgaaagcacaaggacactgg

SEQ ID NO: 302 - DNA, 55 bp, Artificial Sequence
  Synthetic oligonucleotide
tgatccccaacagagagaggtacctgggatcttctgacgtggttcatgtgtgtca

SEQ ID NO: 303 - DNA, 57 bp, Artificial Sequence
  Synthetic oligonucleotide
tccgaattctccaactttcctcccagcaagggtctgcccttgggactcgggcgatga

SEQ ID NO: 304 - DNA, 58 bp, Artificial Sequence
  Synthetic oligonucleotide
tttcctttctttcttccaaactcctgttaatattggtattttgacctcctaactgtgg

SEQ ID NO: 305 - DNA, 55 bp, Artificial Sequence
  Synthetic oligonucleotide
acatagaaggtgttcagtaaatatttcctgactgtaggagttgatgaatgtgcgt

SEQ ID NO: 306 - DNA, 55 bp, Artificial Sequence
  Synthetic oligonucleotide
tcatggccggtggccggttctcaccccttttgcttctaacagacacgcagagcga

SEQ ID NO: 307 - DNA, 56 bp, Artificial Sequence
  Synthetic oligonucleotide
ggagatactgacaattgcaagttgggctgatatgtatgaaaacagaaaatacggca

SEQ ID NO: 308 - DNA, 56 bp, Artificial Sequence
  Synthetic oligonucleotide
taacaaagactagcttatactacccacgctttcctgtcatttttcttttcggttca

SEQ ID NO: 309 - DNA, 56 bp, Artificial Sequence
  Synthetic oligonucleotide
aacattttgttttataatctgcgtctgataatactgatatacaaactctgctctag

SEQ ID NO: 310 - DNA, 57 bp, Artificial Sequence
  Synthetic oligonucleotide
agatggtgaagtaaagatgaataacatgaagcacgtttgaatgctattggcttctga

SEQ ID NO: 311 - DNA, 57 bp, Artificial Sequence
  Synthetic oligonucleotide
tagtgatatttcaatacatataatgtatagttatcagtgtaattagcataaagtaca

SEQ ID NO: 312 - DNA, 57 bp, Artificial Sequence
  Synthetic oligonucleotide
ctttctctaggtgccgtacatgttagtgggggctccttatttcctggattgaagctc

SEQ ID NO: 313 - DNA, 57 bp, Artificial Sequence
  Synthetic oligonucleotide
aactctcagtttgggccgctgctctccagttgcctggagttttaagtcttagacgac

SEQ ID NO: 314 - DNA, 55 bp, Artificial Sequence
  Synthetic oligonucleotide
atggccaagccttggctgttgagtaggcagtgcccagttgtgctgtatggttgga

SEQ ID NO: 315 - DNA, 55 bp, Artificial Sequence
  Synthetic oligonucleotide
tcccgggctccgcatgctgcagcacgtggttcaggatgcgctgctccttgattca

SEQ ID NO: 316 - DNA, 51 bp, Artificial Sequence
  Synthetic oligonucleotide
gaaggaccccagctccaccaaccaacaaaggcacggtccgtcccacccacc

SEQ ID NO: 317 - DNA, 56 bp, Artificial Sequence
  Synthetic oligonucleotide
gaaatagaccctcgacagacccaaaggggcccacatgatgcggtggtgacgttaga

SEQ ID NO: 318 - DNA, 60 bp, Artificial Sequence
  Synthetic oligonucleotide
cccagattttgctaatccatacagttgactggacatgaactcattttggtgagacacata

SEQ ID NO: 319 - DNA, 62 bp, Artificial Sequence
  Synthetic oligonucleotide
attctgaaaggaatgaaaatggggtttaaatgtctttaaggtccagaggggtaactatactt

SEQ ID NO: 320 - DNA, 57 bp, Artificial Sequence
  Synthetic oligonucleotide
attctgaagatttatcatgaaaaaaaaagaatgtacaatcttttattaataaccgtc

SEQ ID NO: 321 - DNA, 59 bp, Artificial Sequence
  Synthetic oligonucleotide
caacctgcccctccctgacccggggcccccttttctccagggcccagtcttgaatggaa

SEQ ID NO: 322 - DNA, 62 bp, Artificial Sequence
  Synthetic oligonucleotide
gttgacttcttttaaaatatgatcttcacaattatcatccaatcaaattgttagacgatgct

SEQ ID NO: 323 - DNA, 56 bp, Artificial Sequence
  Synthetic oligonucleotide
atagctttaccattttaccttgctcaatacacaccccagacaaagagacacactca

SEQ ID NO: 324 - DNA, 56 bp, Artificial Sequence
  Synthetic oligonucleotide
tttgttagcagggttggatctaaccagtgatgtgtggcatgtcagtgtgaatcctt

SEQ ID NO: 325 - DNA, 58 bp, Artificial Sequence
  Synthetic oligonucleotide
cctcgttacctgcttctcatctgtgatgctcccctgatctctgcaacattcataatga

SEQ ID NO: 326 - DNA, 59 bp, Artificial Sequence
  Synthetic oligonucleotide
gcctggggccgggcggcaggggcgcgcagggtgggggcccagaggcctctttagacatg

SEQ ID NO: 327 - DNA, 51 bp, Artificial Sequence
  Synthetic oligonucleotide
gagagagggtgctaggctgctggcccagcaaggcgtcaagggaagcaggga

SEQ ID NO: 328 - DNA, 56 bp, Artificial Sequence
  Synthetic oligonucleotide
ggggttgggggggtggtgttgaggtatgtgtaaggctattgctcatgatatcgccg

SEQ ID NO: 329 - DNA, 60 bp, Artificial Sequence
  Synthetic oligonucleotide
aggcgggaacataaactaacaaaaaagtatgtcatagcacagtgacatgttgagcaacct

SEQ ID NO: 330 - DNA, 51 bp, Artificial Sequence
  Synthetic oligonucleotide
tgatgggagcacaccccccaatgaccctgcccccgcacctgcaaaagccct

SEQ ID NO: 331 - DNA, 56 bp, Artificial Sequence
  Synthetic oligonucleotide
ttaaagcacattaaagctcattagccactatgtcaaggccttatctaatcgattca

SEQ ID NO: 332 - DNA, 59 bp, Artificial Sequence
  Synthetic oligonucleotide
tagtatatcatataaaaataaagacatcacccaatataaaaacatcaccctcggactaa

SEQ ID NO: 333 - DNA, 58 bp, Artificial Sequence
  Synthetic oligonucleotide
atgttgaactcttttgtcaaaagccccttgttgggaaagtctagagacttaatggtct

SEQ ID NO: 334 - DNA, 59 bp, Artificial Sequence
  Synthetic oligonucleotide
aaaccgtatgtgatctagcaatggaggagagggtgagaagtccccagtacgagcataac

SEQ ID NO: 335 - DNA, 60 bp, Artificial Sequence
  Synthetic oligonucleotide
tcaaatttcccgtgatcgttactgcccatttcccaaaatatctcattgcatggttgggtt

SEQ ID NO: 336 - DNA, 56 bp, Artificial Sequence
  Synthetic oligonucleotide
ggtcatgataagtaagcagtgaaacaaagtagacgtatgaatcagtaaaaaatcga

SEQ ID NO: 337 - DNA, 61 bp, Artificial Sequence
  Synthetic oligonucleotide
cttcactcgcagtaaatgtctatttctcctgttttattaaaaaggttgagatagtctcact

SEQ ID NO: 338 - DNA, 60 bp, Artificial Sequence
  Synthetic oligonucleotide
ctctgcccacggtatacctgggagagtgcaggtctttcagaaaggtgagtctattattac

SEQ ID NO: 339 - DNA, 61 bp, Artificial Sequence
  Synthetic oligonucleotide
tgatcatatggtttttgtttttaattctgtttatgtggtgaatcacacttcaccgaataag

SEQ ID NO: 340 - DNA, 58 bp, Artificial Sequence
  Synthetic oligonucleotide
agatagatgacttagaggcccttgggtgtaacagtgagtcagtcttcccactcttcat

SEQ ID NO: 341 - DNA, 66 bp, Artificial Sequence
  Synthetic oligonucleotide
aatcttcataaaacctcagtgaatactcttttttactgttaaaaaaatctagtaatctattatagt

SEQ ID NO: 342 - DNA, 60 bp, Artificial Sequence
  Synthetic oligonucleotide
cttgcttatgaacactaatttcatatataaaacagaaaatttattgtgatggatcatata

SEQ ID NO: 343 - DNA, 43 bp, Artificial Sequence
  Synthetic oligonucleotide
tgcttcgagctccttggaatcaccaacaaacatgccttctcct

SEQ ID NO: 344 - DNA, 49 bp, Artificial Sequence
  Synthetic oligonucleotide
tacactcttggaagtgtgaccaggacatggatgaggacgatggcagaga

SEQ ID NO: 345 - DNA, 41 bp, Artificial Sequence
  Synthetic oligonucleotide
tatgatcttgtatatagacggtaaaataaacaccaagacgt

SEQ ID NO: 346 - DNA, 44 bp, Artificial Sequence
  Synthetic oligonucleotide
tccgtttcatcaattcatacagaacaattccaaatgcatataca

SEQ ID NO: 347 - DNA, 44 bp, Artificial Sequence
  Synthetic oligonucleotide
ggttatcaaaatcatcattttcatctctgtatatggatgcctgt

SEQ ID NO: 348 - DNA, 50 bp, Artificial Sequence
  Synthetic oligonucleotide
tgtgtattagtagaagctttagtagattatgctggtattacggtggaagg

SEQ ID NO: 349 - DNA, 44 bp, Artificial Sequence
  Synthetic oligonucleotide
tagtcgaaagtgacccaggtgagttttgtttcacatgataacca

SEQ ID NO: 350 - DNA, 43 bp, Artificial Sequence
  Synthetic oligonucleotide
cgaagcgcaggagagccaggatgtcagcgggagccgcccggcg

SEQ ID NO: 351 - DNA, 45 bp, Artificial Sequence
  Synthetic oligonucleotide
tgcaataaacaactcaccctgaaggccaaatgatgtcatagtgca

SEQ ID NO: 352 - DNA, 43 bp, Artificial Sequence
  Synthetic oligonucleotide
tcggccaacacaacaacaccaaggtcagtccctgcagatcaca

SEQ ID NO: 353 - DNA, 42 bp, Artificial Sequence
  Synthetic oligonucleotide
acgcaagacaagggcaacgagccctcattccgtggagtgtct

SEQ ID NO: 354 - DNA, 42 bp, Artificial Sequence
  Synthetic oligonucleotide
tgtccgcagtctgaatgagttatgttgtggttcccccaccat

SEQ ID NO: 355 - DNA, 40 bp, Artificial Sequence
  Synthetic oligonucleotide
aagcggtgtagctggtctcttggtccttgggatgcgcatg

SEQ ID NO: 356 - DNA, 45 bp, Artificial Sequence
  Synthetic oligonucleotide
tacattggccagctgcggagccattcagccggacgtcgtgcactt

SEQ ID NO: 357 - DNA, 45 bp, Artificial Sequence
  Synthetic oligonucleotide
agcaggcaccttggctctgtctgaaggccagtgacgcggtgacag

SEQ ID NO: 358 - DNA, 48 bp, Artificial Sequence
  Synthetic oligonucleotide
tcaagcatagtagagtggtggatccagacaccatcagatgccagagaa

SEQ ID NO: 359 - DNA, 48 bp, Artificial Sequence
  Synthetic oligonucleotide
catgtattaagacagtcgtcatctggaaagaaacatcaacatgaggca

SEQ ID NO: 360 - DNA, 44 bp, Artificial Sequence
  Synthetic oligonucleotide
gaagactcagctagacggcacgtatccaccgcccgtccatgatg

SEQ ID NO: 361 - DNA, 46 bp, Artificial Sequence
  Synthetic oligonucleotide
caatatccgtcgattcccagatcttaaccgacttgccaagaagttt

SEQ ID NO: 362 - DNA, 43 bp, Artificial Sequence
  Synthetic oligonucleotide
atcataatcttgctttctgatataatttgttttgtaggcccca

SEQ ID NO: 363 - DNA, 43 bp, Artificial Sequence
  Synthetic oligonucleotide
gcccaccacgccctcgtcactggatgccgggcccgacactgtg

SEQ ID NO: 364 - DNA, 43 bp, Artificial Sequence
  Synthetic oligonucleotide
cagccaacatcgagatggcctatgcgcggcaagtgagtgtgat

SEQ ID NO: 365 - DNA, 46 bp, Artificial Sequence
  Synthetic oligonucleotide
agaatatgatgaagagtttattgtttttgtaggagatggacagctt

SEQ ID NO: 366 - DNA, 46 bp, Artificial Sequence
  Synthetic oligonucleotide
tcgatcatagggactttccgggaacttacgtagaatatattggaag

SEQ ID NO: 367 - DNA, 40 bp, Artificial Sequence
  Synthetic oligonucleotide
cgtgtgataagtggttgcttaatgactccctttctttttc

SEQ ID NO: 368 - DNA, 43 bp, Artificial Sequence
  Synthetic oligonucleotide
gagctcttttcacagccacggttttcacaactctgcaagagaa

SEQ ID NO: 369 - DNA, 49 bp, Artificial Sequence
  Synthetic oligonucleotide
cttgcttgctcactccaggagggcaacggggcttctgctgagggcattt

SEQ ID NO: 370 - DNA, 46 bp, Artificial Sequence
  Synthetic oligonucleotide
gcgtgacttcctcttactctctgcctgcaggatgtgcgacgtgtgc

SEQ ID NO: 371 - DNA, 43 bp, Artificial Sequence
  Synthetic oligonucleotide
gctcttgaaaacaataaatcaaccccctgttgttagtgaggac

SEQ ID NO: 372 - DNA, 44 bp, Artificial Sequence
  Synthetic oligonucleotide
tatttgcctgggccccgacaacatggcatccctgacctgaacca

SEQ ID NO: 373 - DNA, 44 bp, Artificial Sequence
  Synthetic oligonucleotide
accactgtgcctacaccacgtgtggctccatgctccgcacagtg

SEQ ID NO: 374 - DNA, 46 bp, Artificial Sequence
  Synthetic oligonucleotide
agccataagttctcgatgatgtgtggcagagggaaggccctcacac

SEQ ID NO: 375 - DNA, 43 bp, Artificial Sequence
  Synthetic oligonucleotide
actgatgctttctatccccaaattgctcctgggaagataacag

SEQ ID NO: 376 - DNA, 46 bp, Artificial Sequence
  Synthetic oligonucleotide
acaaggtttatttcataggtagcaacattttccaggaagcatttaa

SEQ ID NO: 377 - DNA, 46 bp, Artificial Sequence
  Synthetic oligonucleotide
acctaaccctaacgaggcttacctggaggaaggaatggcactgcac

SEQ ID NO: 378 - DNA, 45 bp, Artificial Sequence
  Synthetic oligonucleotide
agcttggctcacagcccaaacttcaacaccaaaaatacttttcca

SEQ ID NO: 379 - DNA, 44 bp, Artificial Sequence
  Synthetic oligonucleotide
gactccccactggctcactctcgaattctgtcttgaacgaggac

SEQ ID NO: 380 - DNA, 43 bp, Artificial Sequence
  Synthetic oligonucleotide
ttcgttctctcctcagctcaaaacccttcagtggcactccatt

SEQ ID NO: 381 - DNA, 46 bp, Artificial Sequence
  Synthetic oligonucleotide
ctctaatgttctctaaactgacaactcacgcctgcccacgtcgggc

SEQ ID NO: 382 - DNA, 44 bp, Artificial Sequence
  Synthetic oligonucleotide
tgtcgtgcacatggcaagcccggtcttccctgggagttcccaac

SEQ ID NO: 383 - DNA, 45 bp, Artificial Sequence
  Synthetic oligonucleotide
tctctcctaagagacaaaaatgttataccgcatatagaaaacaaa

SEQ ID NO: 384 - DNA, 42 bp, Artificial Sequence
  Synthetic oligonucleotide
acggagagctcttctgtcttggttgtggtcccacagaacatt

SEQ ID NO: 385 - DNA, 47 bp, Artificial Sequence
  Synthetic oligonucleotide
ctgactataaagggtgaatgtttacagttcctgaagcccctgtgggc

SEQ ID NO: 386 - DNA, 40 bp, Artificial Sequence
  Synthetic oligonucleotide
gaggatggtatgtgtttctgagcgcagatagttgttctag

SEQ ID NO: 387 - DNA, 46 bp, Artificial Sequence
  Synthetic oligonucleotide
aatctcattcccagttactactcccaggattacaaacagaattgct

SEQ ID NO: 388 - DNA, 41 bp, Artificial Sequence
  Synthetic oligonucleotide
tccataacaaaaaacaatagagcttaaacaaaaatgaaagg

SEQ ID NO: 389 - DNA, 46 bp, Artificial Sequence
  Synthetic oligonucleotide
gtgatggcaaggggaccatcttaataatggcacagaggaaacatgt

SEQ ID NO: 390 - DNA, 45 bp, Artificial Sequence
  Synthetic oligonucleotide
caagctagctcacgggaccttatggatcctggctccagtgagaca

SEQ ID NO: 391 - DNA, 44 bp, Artificial Sequence
  Synthetic oligonucleotide
tactctgaaaaatcactccatgttattctaagaaaccgtaagtc

SEQ ID NO: 392 - DNA, 42 bp, Artificial Sequence
  Synthetic oligonucleotide
atggtctctcaactgatgtatccatttcattgttgacagcca

SEQ ID NO: 393 - DNA, 40 bp, Artificial Sequence
  Synthetic oligonucleotide
ctgtgccatttcagagtgagatagtgaggtgatcatgggc

SEQ ID NO: 394 - DNA, 44 bp, Artificial Sequence
  Synthetic oligonucleotide
tgcctttcaggtagggacggttcccggaggatgctcagcaccat

SEQ ID NO: 395 - DNA, 45 bp, Artificial Sequence
  Synthetic oligonucleotide
caatcgtaaaggcacctctaacactgtggtcaggagcccagattt

SEQ ID NO: 396 - DNA, 46 bp, Artificial Sequence
  Synthetic oligonucleotide
cacggatggataatttctattattggaagaggcaataaaggacctg

SEQ ID NO: 397 - DNA, 46 bp, Artificial Sequence
  Synthetic oligonucleotide
attacataactaagcatcacctgccccagctgtgatgggctgtaac

SEQ ID NO: 398 - DNA, 39 bp, Artificial Sequence
  Synthetic oligonucleotide
aaccctgtgttctcagcagacgtttaggtggccctgcct

SEQ ID NO: 399 - DNA, 42 bp, Artificial Sequence
  Synthetic oligonucleotide
tactactcatgaagcatgtggcctgtactttctaaacattcc

SEQ ID NO: 400 - DNA, 42 bp, Artificial Sequence
  Synthetic oligonucleotide
tccgaagtttgtctcctcatcaaatctaccagcctacttgca

SEQ ID NO: 401 - DNA, 43 bp, Artificial Sequence
  Synthetic oligonucleotide
acgcgtgactcggcacgcaccgtagcaaaggtttgcacagaaa

SEQ ID NO: 402 - DNA, 47 bp, Artificial Sequence
  Synthetic oligonucleotide
tagactaatgtgcctttctaagtcaaagctcaaactctctacacttg

SEQ ID NO: 403 - DNA, 48 bp, Artificial Sequence
  Synthetic oligonucleotide
ctgcatgtactgttgattcatgagtgaattttattagttactgttgat

SEQ ID NO: 404 - DNA, 42 bp, Artificial Sequence
  Synthetic oligonucleotide
ctaccgtcttaaaatatttaatgcaacttgataaaaatcaaa

SEQ ID NO: 405 - DNA, 47 bp, Artificial Sequence
  Synthetic oligonucleotide
tggcttatgcccacaactattttcatgagagactctgtgagatcctg

SEQ ID NO: 406 - DNA, 44 bp, Artificial Sequence
  Synthetic oligonucleotide
ttaggatatggtcaataatgtttgggccacagaagggcatctga

SEQ ID NO: 407 - DNA, 44 bp, Artificial Sequence
  Synthetic oligonucleotide
aatcgaaagccacctgcatttacttctgcaagcctataatttac

SEQ ID NO: 408 - DNA, 50 bp, Artificial Sequence
  Synthetic oligonucleotide
aagagtatgggggccaaggtggcaccattagtcttcttccacttttctgg

SEQ ID NO: 409 - DNA, 51 bp, Artificial Sequence
  Synthetic oligonucleotide
atgaataattcacttaggcccttccccaccttatgcctcttaccagtaggg

SEQ ID NO: 410 - DNA, 42 bp, Artificial Sequence
  Synthetic oligonucleotide
tggatccaattactgatttgttatggttttgatttttttttc

SEQ ID NO: 411 - DNA, 41 bp, Artificial Sequence
  Synthetic oligonucleotide
agcgctcttagttttaagatcataaaagatattgcttgtaa

SEQ ID NO: 412 - DNA, 42 bp, Artificial Sequence
  Synthetic oligonucleotide
gaactcaactttgctgagagtacgtatcatgactgcatactg

SEQ ID NO: 413 - DNA, 43 bp, Artificial Sequence
  Synthetic oligonucleotide
tctaaggttgcatctgcccccgaggctcctctacagagctcag

SEQ ID NO: 414 - DNA, 41 bp, Artificial Sequence
  Synthetic oligonucleotide
tctccgaattgaaagaagttctgaagaaatattaaaagtgc

SEQ ID NO: 415 - DNA, 45 bp, Artificial Sequence
  Synthetic oligonucleotide
ccagtgtccttgtgctttcatgtctcttgggcagatacctaggag

SEQ ID NO: 416 - DNA, 44 bp, Artificial Sequence
  Synthetic oligonucleotide
tgacacacatgaaccacgtcagaagatcccaggtacctctctct

SEQ ID NO: 417 - DNA, 45 bp, Artificial Sequence
  Synthetic oligonucleotide
tcatcgcccgagtcccaagggcagacccttgctgggaggaaagtt

SEQ ID NO: 418 - DNA, 44 bp, Artificial Sequence
  Synthetic oligonucleotide
ccacagttaggaggtcaaaataccaatattaacaggagtttgga

SEQ ID NO: 419 - DNA, 42 bp, Artificial Sequence
  Synthetic oligonucleotide
acgcacattcatcaactcctacagtcaggaaatatttactga

SEQ ID NO: 420 - DNA, 46 bp, Artificial Sequence
  Synthetic oligonucleotide
tcgctctgcgtgtctgttagaagcaaaaggggtgagaaccggccac

SEQ ID NO: 421 - DNA, 43 bp, Artificial Sequence
  Synthetic oligonucleotide
tgccgtattttctgttttcatacatatcagcccaacttgcaat

SEQ ID NO: 422 - DNA, 42 bp, Artificial Sequence
  Synthetic oligonucleotide
tgaaccgaaaagaaaaatgacaggaaagcgtgggtagtataa

SEQ ID NO: 423 - DNA, 39 bp, Artificial Sequence
  Synthetic oligonucleotide
ctagagcagagtttgtatatcagtattatcagacgcaga

SEQ ID NO: 424 - DNA, 44 bp, Artificial Sequence
  Synthetic oligonucleotide
tcagaagccaatagcattcaaacgtgcttcatgttattcatctt

SEQ ID NO: 425 - DNA, 41 bp, Artificial Sequence
  Synthetic oligonucleotide
tgtactttatgctaattacactgataactatacattatatg

SEQ ID NO: 426 - DNA, 44 bp, Artificial Sequence
  Synthetic oligonucleotide
gagcttcaatccaggaaataaggagcccccactaacatgtacgg

SEQ ID NO: 427 - DNA, 44 bp, Artificial Sequence
  Synthetic oligonucleotide
gtcgtctaagacttaaaactccaggcaactggagagcagcggcc

SEQ ID NO: 428 - DNA, 45 bp, Artificial Sequence
  Synthetic oligonucleotide
tccaaccatacagcacaactgggcactgcctactcaacagccaag

SEQ ID NO: 429 - DNA, 47 bp, Artificial Sequence
  Synthetic oligonucleotide
tgaatcaaggagcagcgcatcctgaaccacgtgctgcagcatgcgga

SEQ ID NO: 430 - DNA, 41 bp, Artificial Sequence
  Synthetic oligonucleotide
ggtgggtgggacggaccgtgcctttgttggttggtggagct

SEQ ID NO: 431 - DNA, 43 bp, Artificial Sequence
  Synthetic oligonucleotide
tctaacgtcaccaccgcatcatgtgggcccctttgggtctgtc

SEQ ID NO: 432 - DNA, 45 bp, Artificial Sequence
  Synthetic oligonucleotide
gtgtctcaccaaaatgagttcatgtccagtcaactgtatggatta

SEQ ID NO: 433 - DNA, 46 bp, Artificial Sequence
  Synthetic oligonucleotide
gtatagttacccctctggaccttaaagacatttaaaccccattttc

SEQ ID NO: 434 - DNA, 41 bp, Artificial Sequence
  Synthetic oligonucleotide
gacggttattaataaaagattgtacattctttttttttcat

SEQ ID NO: 435 - DNA, 49 bp, Artificial Sequence
  Synthetic oligonucleotide
ttccattcaagactgggccctggagaaaagggggccccgggtcagggag

SEQ ID NO: 436 - DNA, 45 bp, Artificial Sequence
  Synthetic oligonucleotide
catcgtctaacaatttgattggatgataattgtgaagatcatatt

SEQ ID NO: 437 - DNA, 41 bp, Artificial Sequence
  Synthetic oligonucleotide
gagtgtgtctctttgtctggggtgtgtattgagcaaggtaa

SEQ ID NO: 438 - DNA, 44 bp, Artificial Sequence
  Synthetic oligonucleotide
aaggattcacactgacatgccacacatcactggttagatccaac

SEQ ID NO: 439 - DNA, 47 bp, Artificial Sequence
  Synthetic oligonucleotide
tcattatgaatgttgcagagatcaggggagcatcacagatgagaagc

SEQ ID NO: 440 - DNA, 48 bp, Artificial Sequence
  Synthetic oligonucleotide
gtctaaagaggcctctgggcccccaccctgcgcgcccctgccgcccgg

SEQ ID NO: 441 - DNA, 39 bp, Artificial Sequence
  Synthetic oligonucleotide
ccctgcttcccttgacgccttgctgggccagcagcctag

SEQ ID NO: 442 - DNA, 46 bp, Artificial Sequence
  Synthetic oligonucleotide
ggcgatatcatgagcaatagccttacacatacctcaacaccacccc

SEQ ID NO: 443 - DNA, 48 bp, Artificial Sequence
  Synthetic oligonucleotide
ttgctcaacatgtcactgtgctatgacatacttttttgttagtttatg

SEQ ID NO: 444 - DNA, 41 bp, Artificial Sequence
  Synthetic oligonucleotide
agggcttttgcaggtgcgggggcagggtcattggggggtgt

SEQ ID NO: 445 - DNA, 41 bp, Artificial Sequence
  Synthetic oligonucleotide
tgaatcgattagataaggccttgacatagtggctaatgagc

SEQ ID NO: 446 - DNA, 36 bp, Artificial Sequence
  Synthetic oligonucleotide
gtccgagggtgatgtttttatattgggtgatgtctt

SEQ ID NO: 447 - DNA, 44 bp, Artificial Sequence
  Synthetic oligonucleotide
agaccattaagtctctagactttcccaacaaggggcttttgaca

SEQ ID NO: 448 - DNA, 45 bp, Artificial Sequence
  Synthetic oligonucleotide
tatgctcgtactggggacttctcaccctctcctccattgctagat

SEQ ID NO: 449 - DNA, 45 bp, Artificial Sequence
  Synthetic oligonucleotide
ccaaccatgcaatgagatattttgggaaatgggcagtaacgatca

SEQ ID NO: 450 - DNA, 41 bp, Artificial Sequence
  Synthetic oligonucleotide
cgattttttactgattcatacgtctactttgtttcactgct

SEQ ID NO: 451 - DNA, 50 bp, Artificial Sequence
  Synthetic oligonucleotide
agtgagactatctcaacctttttaataaaacaggagaaatagacatttac

SEQ ID NO: 452 - DNA, 49 bp, Artificial Sequence
  Synthetic oligonucleotide
taataatagactcacctttctgaaagacctgcactctcccaggtatacc

SEQ ID NO: 453 - DNA, 45 bp, Artificial Sequence
  Synthetic oligonucleotide
tattcggtgaagtgtgattcaccacataaacagaattaaaaacaa

SEQ ID NO: 454 - DNA, 41 bp, Artificial Sequence
  Synthetic oligonucleotide
gaagagtgggaagactgactcactgttacacccaagggcct

SEQ ID NO: 455 - DNA, 51 bp, Artificial Sequence
  Synthetic oligonucleotide
actataatagattactagatttttttaacagtaaaaaagagtattcactga

SEQ ID NO: 456 - DNA, 45 bp, Artificial Sequence
  Synthetic oligonucleotide
tatgatccatcacaataaattttctgttttatatatgaaattagt

SEQ ID NO: 457 - DNA, 54 bp, Artificial Sequence
  Synthetic oligonucleotide
tcgtcggcagcgtcagatgtgtataagagacagccaagcacatggatcagtgtt

SEQ ID NO: 458 - DNA, 55 bp, Artificial Sequence
  Synthetic oligonucleotide
tcgtcggcagcgtcagatgtgtataagagacagagggaagggcatatctggatac

SEQ ID NO: 459 - DNA, 53 bp, Artificial Sequence
  Synthetic oligonucleotide
tcgtcggcagcgtcagatgtgtataagagacagttcacgcttacccaggagtt

SEQ ID NO: 460 - DNA, 58 bp, Artificial Sequence
  Synthetic oligonucleotide
tcgtcggcagcgtcagatgtgtataagagacagaaggtaactgtccagtcatcaattc

SEQ ID NO: 461 - DNA, 55 bp, Artificial Sequence
  Synthetic oligonucleotide
tcgtcggcagcgtcagatgtgtataagagacaggctgtgtagtttctaagggtcg

SEQ ID NO: 462 - DNA, 52 bp, Artificial Sequence
  Synthetic oligonucleotide
tcgtcggcagcgtcagatgtgtataagagacagcccagacgagtacagctca

SEQ ID NO: 463 - DNA, 55 bp, Artificial Sequence
  Synthetic oligonucleotide
tcgtcggcagcgtcagatgtgtataagagacagagaatcctgatctgactggctt

SEQ ID NO: 464 - DNA, 55 bp, Artificial Sequence
  Synthetic oligonucleotide
tcgtcggcagcgtcagatgtgtataagagacagcttgcccgagttctactacaga

SEQ ID NO: 465 - DNA, 52 bp, Artificial Sequence
  Synthetic oligonucleotide
tcgtcggcagcgtcagatgtgtataagagacagctggctctgtgcagaactg

SEQ ID NO: 466 - DNA, 50 bp, Artificial Sequence
  Synthetic oligonucleotide
tcgtcggcagcgtcagatgtgtataagagacaggcccagatcgtgtgctc

SEQ ID NO: 467 - DNA, 51 bp, Artificial Sequence
  Synthetic oligonucleotide
tcgtcggcagcgtcagatgtgtataagagacaggctggactggcttcacaa

SEQ ID NO: 468 - DNA, 55 bp, Artificial Sequence
  Synthetic oligonucleotide
tcgtcggcagcgtcagatgtgtataagagacagctcctcgtggatccaaaattgc

SEQ ID NO: 469 - DNA, 51 bp, Artificial Sequence
  Synthetic oligonucleotide
tcgtcggcagcgtcagatgtgtataagagacagggtttcaagccctctgca

SEQ ID NO: 470 - DNA, 51 bp, Artificial Sequence
  Synthetic oligonucleotide
tcgtcggcagcgtcagatgtgtataagagacagcagcccaagccattgtct

SEQ ID NO: 471 - DNA, 57 bp, Artificial Sequence
  Synthetic oligonucleotide
tcgtcggcagcgtcagatgtgtataagagacagagcctaagcaatataaatggctgc

SEQ ID NO: 472 - DNA, 54 bp, Artificial Sequence
  Synthetic oligonucleotide
tcgtcggcagcgtcagatgtgtataagagacagcagagtagagtggtggatcca

SEQ ID NO: 473 - DNA, 51 bp, Artificial Sequence
  Synthetic oligonucleotide
tcgtcggcagcgtcagatgtgtataagagacagtgaacacagcccacctca

SEQ ID NO: 474 - DNA, 52 bp, Artificial Sequence
  Synthetic oligonucleotide
tcgtcggcagcgtcagatgtgtataagagacagctctgcactccatgccaac

SEQ ID NO: 475 - DNA, 56 bp, Artificial Sequence
  Synthetic oligonucleotide
tcgtcggcagcgtcagatgtgtataagagacagtggaagcttttgtagaagatgca

SEQ ID NO: 476 - DNA, 60 bp, Artificial Sequence
  Synthetic oligonucleotide
tcgtcggcagcgtcagatgtgtataagagacagcagtgtacagtttaggactaacaatcc

SEQ ID NO: 477 - DNA, 53 bp, Artificial Sequence
  Synthetic oligonucleotide
tcgtcggcagcgtcagatgtgtataagagacaggtcatcagtggtgaggagga

SEQ ID NO: 478 - DNA, 55 bp, Artificial Sequence
  Synthetic oligonucleotide
tcgtcggcagcgtcagatgtgtataagagacagccaagctacatcagtgatgtgg

SEQ ID NO: 479 - DNA, 52 bp, Artificial Sequence
  Synthetic oligonucleotide
tcgtcggcagcgtcagatgtgtataagagacagtgtgcaggcacttaccaag

SEQ ID NO: 480 - DNA, 52 bp, Artificial Sequence
  Synthetic oligonucleotide
tcgtcggcagcgtcagatgtgtataagagacaggaagccaggcctgaagaaa

SEQ ID NO: 481 - DNA, 56 bp, Artificial Sequence
  Synthetic oligonucleotide
tcgtcggcagcgtcagatgtgtataagagacagactgaagcagatgttgaacaaca

SEQ ID NO: 482 - DNA, 54 bp, Artificial Sequence
  Synthetic oligonucleotide
tcgtcggcagcgtcagatgtgtataagagacagtcattggcctcgtttttcagt

SEQ ID NO: 483 - DNA, 50 bp, Artificial Sequence
  Synthetic oligonucleotide
tcgtcggcagcgtcagatgtgtataagagacagcctggttgcttggcaca

SEQ ID NO: 484 - DNA, 53 bp, Artificial Sequence
  Synthetic oligonucleotide
tcgtcggcagcgtcagatgtgtataagagacagctccctcaagacctacgtga

SEQ ID NO: 485 - DNA, 56 bp, Artificial Sequence
  Synthetic oligonucleotide
tcgtcggcagcgtcagatgtgtataagagacaggttaaagacggcacttccaacag

SEQ ID NO: 486 - DNA, 50 bp, Artificial Sequence
  Synthetic oligonucleotide
tcgtcggcagcgtcagatgtgtataagagacagtcctccgtggctctccc

SEQ ID NO: 487 - DNA, 52 bp, Artificial Sequence
  Synthetic oligonucleotide
tcgtcggcagcgtcagatgtgtataagagacagagcttggggacacctctga

SEQ ID NO: 488 - DNA, 51 bp, Artificial Sequence
  Synthetic oligonucleotide
tcgtcggcagcgtcagatgtgtataagagacaggatggttccagctgcgct

SEQ ID NO: 489 - DNA, 61 bp, Artificial Sequence
  Synthetic oligonucleotide
tcgtcggcagcgtcagatgtgtataagagacagtccgatataagttaacaatgcaatgtca

SEQ ID NO: 490 - DNA, 69 bp, Artificial Sequence
  Synthetic oligonucleotide
tcgtcggcagcgtcagatgtgtataagagacagtgatcttatttatatattttcagtcatttgtcctac

SEQ ID NO: 491 - DNA, 58 bp, Artificial Sequence
  Synthetic oligonucleotide
tcgtcggcagcgtcagatgtgtataagagacaggctccaacatttcatccaggatttg

SEQ ID NO: 492 - DNA, 58 bp, Artificial Sequence
  Synthetic oligonucleotide
tcgtcggcagcgtcagatgtgtataagagacaggccattacacctaagcaccatctac

SEQ ID NO: 493 - DNA, 59 bp, Artificial Sequence
  Synthetic oligonucleotide
tcgtcggcagcgtcagatgtgtataagagacagaaagctaaagcagagaatgaagttga

SEQ ID NO: 494 - DNA, 60 bp, Artificial Sequence
  Synthetic oligonucleotide
tcgtcggcagcgtcagatgtgtataagagacagaatatcatgtcctatttctcctcagct

SEQ ID NO: 495 - DNA, 57 bp, Artificial Sequence
  Synthetic oligonucleotide
tcgtcggcagcgtcagatgtgtataagagacagggagctgtgacaatgaaaatgcag

SEQ ID NO: 496 - DNA, 52 bp, Artificial Sequence
  Synthetic oligonucleotide
tcgtcggcagcgtcagatgtgtataagagacagccgtcaccgtggagtttcc

SEQ ID NO: 497 - DNA, 62 bp, Artificial Sequence
  Synthetic oligonucleotide
tcgtcggcagcgtcagatgtgtataagagacagtggaagagcttacatttaagtgattactg

SEQ ID NO: 498 - DNA, 57 bp, Artificial Sequence
  Synthetic oligonucleotide
tcgtcggcagcgtcagatgtgtataagagacagaaagtggtggtttttaaccccttc

SEQ ID NO: 499 - DNA, 60 bp, Artificial Sequence
  Synthetic oligonucleotide
tcgtcggcagcgtcagatgtgtataagagacaggcctatagatggcaaattaagagagca

SEQ ID NO: 500 - DNA, 64 bp, Artificial Sequence
  Synthetic oligonucleotide
tcgtcggcagcgtcagatgtgtataagagacagaaaaagtgaatcaatagttgtactagtgcta

SEQ ID NO: 501 - DNA, 54 bp, Artificial Sequence
  Synthetic oligonucleotide
tcgtcggcagcgtcagatgtgtataagagacagatgggaagggtacgatgttcc

SEQ ID NO: 502 - DNA, 64 bp, Artificial Sequence
  Synthetic oligonucleotide
tcgtcggcagcgtcagatgtgtataagagacaggattaggataattttccagctcaaagaaaat

SEQ ID NO: 503 - DNA, 66 bp, Artificial Sequence
  Synthetic oligonucleotide
tcgtcggcagcgtcagatgtgtataagagacagcctctaaaactagagtgcctatagaatttattg

SEQ ID NO: 504 - DNA, 61 bp, Artificial Sequence
  Synthetic oligonucleotide
tcgtcggcagcgtcagatgtgtataagagacagagtatttagttaacggttgttttacgct

SEQ ID NO: 505 - DNA, 62 bp, Artificial Sequence
  Synthetic oligonucleotide
tcgtcggcagcgtcagatgtgtataagagacaggaattttgatgaaaacattcctgctatca

SEQ ID NO: 506 - DNA, 61 bp, Artificial Sequence
  Synthetic oligonucleotide
tcgtcggcagcgtcagatgtgtataagagacagcacagtgttctacggtatacaagtatct

SEQ ID NO: 507 - DNA, 66 bp, Artificial Sequence
  Synthetic oligonucleotide
tcgtcggcagcgtcagatgtgtataagagacaggctaccttatagtcttccctagcttaataattt

SEQ ID NO: 508 - DNA, 48 bp, Artificial Sequence
  Synthetic oligonucleotide
tcgtcggcagcgtcagatgtgtataagagacaggcagggtggctgcgt

SEQ ID NO: 509 - DNA, 59 bp, Artificial Sequence
  Synthetic oligonucleotide
tcgtcggcagcgtcagatgtgtataagagacaggcttggaatgaaatccctatccctat

SEQ ID NO: 510 - DNA, 52 bp, Artificial Sequence
  Synthetic oligonucleotide
tcgtcggcagcgtcagatgtgtataagagacagcagtggtcctgacgttcgg

SEQ ID NO: 511 - DNA, 62 bp, Artificial Sequence
  Synthetic oligonucleotide
tcgtcggcagcgtcagatgtgtataagagacagcctaatacattaaagcagtcacttttcct

SEQ ID NO: 512 - DNA, 50 bp, Artificial Sequence
  Synthetic oligonucleotide
tcgtcggcagcgtcagatgtgtataagagacagggctcacgtcatgggca

SEQ ID NO: 513 - DNA, 63 bp, Artificial Sequence
  Synthetic oligonucleotide
tcgtcggcagcgtcagatgtgtataagagacagagaactcaaacaagatttaaggtctagaaa

SEQ ID NO: 514 - DNA, 54 bp, Artificial Sequence
  Synthetic oligonucleotide
tcgtcggcagcgtcagatgtgtataagagacagcctccactcaaagtttctggc

SEQ ID NO: 515 - DNA, 55 bp, Artificial Sequence
  Synthetic oligonucleotide
tcgtcggcagcgtcagatgtgtataagagacagtggcacagactttattggctct

SEQ ID NO: 516 - DNA, 58 bp, Artificial Sequence
  Synthetic oligonucleotide
tcgtcggcagcgtcagatgtgtataagagacagcagagatcatttctattgccacagg

SEQ ID NO: 517 - DNA, 58 bp, Artificial Sequence
  Synthetic oligonucleotide
tcgtcggcagcgtcagatgtgtataagagacaggtaagcctagtgcccagtatatcat

SEQ ID NO: 518 - DNA, 63 bp, Artificial Sequence
  Synthetic oligonucleotide
tcgtcggcagcgtcagatgtgtataagagacaggctagtgtacgatatgtgtgtattgattaa

SEQ ID NO: 519 - DNA, 55 bp, Artificial Sequence
  Synthetic oligonucleotide
tcgtcggcagcgtcagatgtgtataagagacagaccctcctgcttatgtggttac

SEQ ID NO: 520 - DNA, 52 bp, Artificial Sequence
  Synthetic oligonucleotide
tcgtcggcagcgtcagatgtgtataagagacagccctgggtcacacacaaca

SEQ ID NO: 521 - DNA, 58 bp, Artificial Sequence
  Synthetic oligonucleotide
tcgtcggcagcgtcagatgtgtataagagacagaagtgagtgggaacagtcatattga

SEQ ID NO: 522 - DNA, 55 bp, Artificial Sequence
  Synthetic oligonucleotide
tcgtcggcagcgtcagatgtgtataagagacaggcagataggtacagaggcgtct

SEQ ID NO: 523 - DNA, 56 bp, Artificial Sequence
  Synthetic oligonucleotide
tcgtcggcagcgtcagatgtgtataagagacagttgactttccagttccccactta

SEQ ID NO: 524 - DNA, 65 bp, Artificial Sequence
  Synthetic oligonucleotide
tcgtcggcagcgtcagatgtgtataagagacaggttcttgggaagtttttgattactaattcaat

SEQ ID NO: 525 - DNA, 65 bp, Artificial Sequence
  Synthetic oligonucleotide
tcgtcggcagcgtcagatgtgtataagagacagctttttatatattgcacactctaaaaagaggt

SEQ ID NO: 526 - DNA, 62 bp, Artificial Sequence
  Synthetic oligonucleotide
tcgtcggcagcgtcagatgtgtataagagacaggggaaaaacaaaattgtctcaaaaaatgt

SEQ ID NO: 527 - DNA, 51 bp, Artificial Sequence
  Synthetic oligonucleotide
tcgtcggcagcgtcagatgtgtataagagacaggggcggatgccattgagt

SEQ ID NO: 528 - DNA, 66 bp, Artificial Sequence
  Synthetic oligonucleotide
tcgtcggcagcgtcagatgtgtataagagacaggcacttctaagttattatgatagagtgatgtac

SEQ ID NO: 529 - DNA, 58 bp, Artificial Sequence
  Synthetic oligonucleotide
tcgtcggcagcgtcagatgtgtataagagacaggcagtaaatcaacccgctataaacg

SEQ ID NO: 530 - DNA, 55 bp, Artificial Sequence
  Synthetic oligonucleotide
tcgtcggcagcgtcagatgtgtataagagacaggttgcccttgccaatagtgaaa

SEQ ID NO: 531 - DNA, 62 bp, Artificial Sequence
  Synthetic oligonucleotide
tcgtcggcagcgtcagatgtgtataagagacagcagctagttctatatttacagacagagac

SEQ ID NO: 532 - DNA, 60 bp, Artificial Sequence
  Synthetic oligonucleotide
tcgtcggcagcgtcagatgtgtataagagacagaatcctgtatctagtgccaatctagaa

SEQ ID NO: 533 - DNA, 60 bp, Artificial Sequence
  Synthetic oligonucleotide
tcgtcggcagcgtcagatgtgtataagagacagagtatctataatagtgcgtggcacata

SEQ ID NO: 534 - DNA, 49 bp, Artificial Sequence
  Synthetic oligonucleotide
tcgtcggcagcgtcagatgtgtataagagacagggaagacccggcggga

SEQ ID NO: 535 - DNA, 57 bp, Artificial Sequence
  Synthetic oligonucleotide
tcgtcggcagcgtcagatgtgtataagagacagtcctctcctgcttaatgtagtcac

SEQ ID NO: 536 - DNA, 69 bp, Artificial Sequence
  Synthetic oligonucleotide
tcgtcggcagcgtcagatgtgtataagagacagatccttccagagaatacacaaattatatgtatatat

SEQ ID NO: 537 - DNA, 60 bp, Artificial Sequence
  Synthetic oligonucleotide
tcgtcggcagcgtcagatgtgtataagagacagggtacatgaccataataaatcagcagg

SEQ ID NO: 538 - DNA, 64 bp, Artificial Sequence
  Synthetic oligonucleotide
tcgtcggcagcgtcagatgtgtataagagacagctctctctactgaattttgattttccatttc

SEQ ID NO: 539 - DNA, 64 bp, Artificial Sequence
  Synthetic oligonucleotide
tcgtcggcagcgtcagatgtgtataagagacagacactgagtattcccaatgtaaagaaataat

SEQ ID NO: 540 - DNA, 49 bp, Artificial Sequence
  Synthetic oligonucleotide
tcgtcggcagcgtcagatgtgtataagagacagcctgggccttcgccct

SEQ ID NO: 541 - DNA, 51 bp, Artificial Sequence
  Synthetic oligonucleotide
tcgtcggcagcgtcagatgtgtataagagacagggcccactgcactcacct

SEQ ID NO: 542 - DNA, 60 bp, Artificial Sequence
  Synthetic oligonucleotide
tcgtcggcagcgtcagatgtgtataagagacagcaggtagggaaagatttcttaagtgag

SEQ ID NO: 543 - DNA, 52 bp, Artificial Sequence
  Synthetic oligonucleotide
tcgtcggcagcgtcagatgtgtataagagacagcctgcagcccatccacaac

SEQ ID NO: 544 - DNA, 54 bp, Artificial Sequence
  Synthetic oligonucleotide
tcgtcggcagcgtcagatgtgtataagagacagactgtgagaggctcagaagga

SEQ ID NO: 545 - DNA, 50 bp, Artificial Sequence
  Synthetic oligonucleotide
tcgtcggcagcgtcagatgtgtataagagacagggggtcagcaggtggca

SEQ ID NO: 546 - DNA, 61 bp, Artificial Sequence
  Synthetic oligonucleotide
tcgtcggcagcgtcagatgtgtataagagacaggggagatgaaataagtaccaaaatgagt

SEQ ID NO: 547 - DNA, 54 bp, Artificial Sequence
  Synthetic oligonucleotide
tcgtcggcagcgtcagatgtgtataagagacaggcattgccactttggctttcg

SEQ ID NO: 548 - DNA, 66 bp, Artificial Sequence
  Synthetic oligonucleotide
tcgtcggcagcgtcagatgtgtataagagacagcctctctaaaacttgatgattttaacatgtaat

SEQ ID NO: 549 - DNA, 50 bp, Artificial Sequence
  Synthetic oligonucleotide
tcgtcggcagcgtcagatgtgtataagagacaggttcccacagccgtggt

SEQ ID NO: 550 - DNA, 61 bp, Artificial Sequence
  Synthetic oligonucleotide
tcgtcggcagcgtcagatgtgtataagagacagaaatatatagagccgcacaccaaaaata

SEQ ID NO: 551 - DNA, 56 bp, Artificial Sequence
  Synthetic oligonucleotide
tcgtcggcagcgtcagatgtgtataagagacagaaaaatggggcagaatgttgtca

SEQ ID NO: 552 - DNA, 52 bp, Artificial Sequence
  Synthetic oligonucleotide
tcgtcggcagcgtcagatgtgtataagagacaggagcaagttcggtctggct

SEQ ID NO: 553 - DNA, 64 bp, Artificial Sequence
  Synthetic oligonucleotide
tcgtcggcagcgtcagatgtgtataagagacagcccattgattaaacaaatattcactgagtac

SEQ ID NO: 554 - DNA, 50 bp, Artificial Sequence
  Synthetic oligonucleotide
tcgtcggcagcgtcagatgtgtataagagacagcttcgggcctctggacc

SEQ ID NO: 555 - DNA, 55 bp, Artificial Sequence
  Synthetic oligonucleotide
tcgtcggcagcgtcagatgtgtataagagacagatctcccgtctcatcctgaaac

SEQ ID NO: 556 - DNA, 56 bp, Artificial Sequence
  Synthetic oligonucleotide
tcgtcggcagcgtcagatgtgtataagagacaggtggaaggcatactgagtgaact

SEQ ID NO: 557 - DNA, 63 bp, Artificial Sequence
  Synthetic oligonucleotide
tcgtcggcagcgtcagatgtgtataagagacaggtgtctccattacattgcttgttttaattt

SEQ ID NO: 558 - DNA, 54 bp, Artificial Sequence
  Synthetic oligonucleotide
tcgtcggcagcgtcagatgtgtataagagacagtctgacatttcacagctggca

SEQ ID NO: 559 - DNA, 53 bp, Artificial Sequence
  Synthetic oligonucleotide
tcgtcggcagcgtcagatgtgtataagagacaggctcccaccagctactgtga

SEQ ID NO: 560 - DNA, 56 bp, Artificial Sequence
  Synthetic oligonucleotide
tcgtcggcagcgtcagatgtgtataagagacaggttcacccagaagtcattccgta

SEQ ID NO: 561 - DNA, 57 bp, Artificial Sequence
  Synthetic oligonucleotide
tcgtcggcagcgtcagatgtgtataagagacaggaggcacagtgctttgtatttgat

SEQ ID NO: 562 - DNA, 58 bp, Artificial Sequence
  Synthetic oligonucleotide
tcgtcggcagcgtcagatgtgtataagagacagttagccactacctttttggctacta

SEQ ID NO: 563 - DNA, 55 bp, Artificial Sequence
  Synthetic oligonucleotide
tcgtcggcagcgtcagatgtgtataagagacagcgttttacagcaagcgacagaa

SEQ ID NO: 564 - DNA, 57 bp, Artificial Sequence
  Synthetic oligonucleotide
tcgtcggcagcgtcagatgtgtataagagacagatgtgatgtgctctaggaaaatgc

SEQ ID NO: 565 - DNA, 61 bp, Artificial Sequence
  Synthetic oligonucleotide
tcgtcggcagcgtcagatgtgtataagagacaggccttgtgagaacagactaatacagata

SEQ ID NO: 566 - DNA, 53 bp, Artificial Sequence
  Synthetic oligonucleotide
tcgtcggcagcgtcagatgtgtataagagacagggtaggtgtggtcaggtcga

SEQ ID NO: 567 - DNA, 52 bp, Artificial Sequence
  Synthetic oligonucleotide
tcgtcggcagcgtcagatgtgtataagagacagacctccctcttgggatgca

SEQ ID NO: 568 - DNA, 64 bp, Artificial Sequence
  Synthetic oligonucleotide
tcgtcggcagcgtcagatgtgtataagagacagggagaataatagttaattaatccacgaagca

SEQ ID NO: 569 - DNA, 61 bp, Artificial Sequence
  Synthetic oligonucleotide
tcgtcggcagcgtcagatgtgtataagagacagagagtctcaattattgctcagttaggat

SEQ ID NO: 570 - DNA, 56 bp, Artificial Sequence
  Synthetic oligonucleotide
tcgtcggcagcgtcagatgtgtataagagacagggtgacctgcgttacttgcttat

SEQ ID NO: 571 - DNA, 55 bp, Artificial Sequence
  Synthetic oligonucleotide
gtctcgtgggctcggagatgtgtataagagacaggagacaggaaagggaaggagt

SEQ ID NO: 572 - DNA, 53 bp, Artificial Sequence
  Synthetic oligonucleotide
gtctcgtgggctcggagatgtgtataagagacagtgtctccaggagcagcttc

SEQ ID NO: 573 - DNA, 55 bp, Artificial Sequence
  Synthetic oligonucleotide
gtctcgtgggctcggagatgtgtataagagacagatcaacaacagggaccaggta

SEQ ID NO: 574 - DNA, 56 bp, Artificial Sequence
  Synthetic oligonucleotide
gtctcgtgggctcggagatgtgtataagagacagtgttctaacaggcaccagaagt

SEQ ID NO: 575 - DNA, 53 bp, Artificial Sequence
  Synthetic oligonucleotide
gtctcgtgggctcggagatgtgtataagagacagaccactctggctgcaaagt

SEQ ID NO: 576 - DNA, 65 bp, Artificial Sequence
  Synthetic oligonucleotide
gtctcgtgggctcggagatgtgtataagagacagaagttattgttattcttgatggttcttttga

SEQ ID NO: 577 - DNA, 61 bp, Artificial Sequence
  Synthetic oligonucleotide
gtctcgtgggctcggagatgtgtataagagacaggttccaatgaattcaattatgctgtca

SEQ ID NO: 578 - DNA, 55 bp, Artificial Sequence
  Synthetic oligonucleotide
gtctcgtgggctcggagatgtgtataagagacagcaaatgcgtgtcctcagagtt

SEQ ID NO: 579 - DNA, 57 bp, Artificial Sequence
  Synthetic oligonucleotide
gtctcgtgggctcggagatgtgtataagagacagtcctagtttcgttgattgcaagg

SEQ ID NO: 580 - DNA, 53 bp, Artificial Sequence
  Synthetic oligonucleotide
gtctcgtgggctcggagatgtgtataagagacagtccaccatgggaaacctgg

SEQ ID NO: 581 - DNA, 55 bp, Artificial Sequence
  Synthetic oligonucleotide
gtctcgtgggctcggagatgtgtataagagacagttcacaggggcatgttttagc

SEQ ID NO: 582 - DNA, 54 bp, Artificial Sequence
  Synthetic oligonucleotide
gtctcgtgggctcggagatgtgtataagagacagaaaggcaaagagggctttgg

SEQ ID NO: 583 - DNA, 63 bp, Artificial Sequence
  Synthetic oligonucleotide
gtctcgtgggctcggagatgtgtataagagacagctgatctatgattctaaattttgctgtca

SEQ ID NO: 584 - DNA, 58 bp, Artificial Sequence
  Synthetic oligonucleotide
gtctcgtgggctcggagatgtgtataagagacagaaccttggagataactctgaagga

SEQ ID NO: 585 - DNA, 54 bp, Artificial Sequence
  Synthetic oligonucleotide
gtctcgtgggctcggagatgtgtataagagacaggtctctggaaacagcccttc

SEQ ID NO: 586 - DNA, 57 bp, Artificial Sequence
  Synthetic oligonucleotide
gtctcgtgggctcggagatgtgtataagagacagagattgtgtttatgttcccagca

SEQ ID NO: 587 - DNA, 59 bp, Artificial Sequence
  Synthetic oligonucleotide
gtctcgtgggctcggagatgtgtataagagacagaacaacaacaacagaaaccagttag

SEQ ID NO: 588 - DNA, 56 bp, Artificial Sequence
  Synthetic oligonucleotide
gtctcgtgggctcggagatgtgtataagagacaggcacctttcacaatggttaagg

SEQ ID NO: 589 - DNA, 61 bp, Artificial Sequence
  Synthetic oligonucleotide
gtctcgtgggctcggagatgtgtataagagacagtgatagagtcggtaacaatcttgtaag

SEQ ID NO: 590 - DNA, 53 bp, Artificial Sequence
  Synthetic oligonucleotide
gtctcgtgggctcggagatgtgtataagagacagcccaatttgggccatgagt

SEQ ID NO: 591 - DNA, 52 bp, Artificial Sequence
  Synthetic oligonucleotide
gtctcgtgggctcggagatgtgtataagagacagcagttgtgtccctgacgg

SEQ ID NO: 592 - DNA, 58 bp, Artificial Sequence
  Synthetic oligonucleotide
gtctcgtgggctcggagatgtgtataagagacaggtttccttttactccctagaggtt

SEQ ID NO: 593 - DNA, 57 bp, Artificial Sequence
  Synthetic oligonucleotide
gtctcgtgggctcggagatgtgtataagagacagtcatggtttcatttgtccctaca

SEQ ID NO: 594 - DNA, 51 bp, Artificial Sequence
  Synthetic oligonucleotide
gtctcgtgggctcggagatgtgtataagagacagaggaagaggccgaggtg

SEQ ID NO: 595 - DNA, 56 bp, Artificial Sequence
  Synthetic oligonucleotide
gtctcgtgggctcggagatgtgtataagagacagcccagaacataacgactcaacc

SEQ ID NO: 596 - DNA, 55 bp, Artificial Sequence
  Synthetic oligonucleotide
gtctcgtgggctcggagatgtgtataagagacagcacagggggattatgcttcac

SEQ ID NO: 597 - DNA, 53 bp, Artificial Sequence
  Synthetic oligonucleotide
gtctcgtgggctcggagatgtgtataagagacagtgaaggaaggcctggagaa

SEQ ID NO: 598 - DNA, 56 bp, Artificial Sequence
  Synthetic oligonucleotide
gtctcgtgggctcggagatgtgtataagagacagcgtttctcactggtctcagatg

SEQ ID NO: 599 - DNA, 53 bp, Artificial Sequence
  Synthetic oligonucleotide
gtctcgtgggctcggagatgtgtataagagacagtgacccttgccctggtaga

SEQ ID NO: 600 - DNA, 52 bp, Artificial Sequence
  Synthetic oligonucleotide
gtctcgtgggctcggagatgtgtataagagacagctgccctggagccactag

SEQ ID NO: 601 - DNA, 53 bp, Artificial Sequence
  Synthetic oligonucleotide
gtctcgtgggctcggagatgtgtataagagacagaccacgaacagcagaagca

SEQ ID NO: 602 - DNA, 69 bp, Artificial Sequence
  Synthetic oligonucleotide
gtctcgtgggctcggagatgtgtataagagacagtgtgtatcatcatctctaatttaaagaaaaagtac

SEQ ID NO: 603 - DNA, 50 bp, Artificial Sequence
  Synthetic oligonucleotide
gtctcgtgggctcggagatgtgtataagagacagtggccagccaagggga

SEQ ID NO: 604 - DNA, 57 bp, Artificial Sequence
  Synthetic oligonucleotide
gtctcgtgggctcggagatgtgtataagagacagccgtgtgctccatcttacaatac

SEQ ID NO: 605 - DNA, 52 bp, Artificial Sequence
  Synthetic oligonucleotide
gtctcgtgggctcggagatgtgtataagagacagggcccagcgtgtgtatga

SEQ ID NO: 606 - DNA, 60 bp, Artificial Sequence
  Synthetic oligonucleotide
gtctcgtgggctcggagatgtgtataagagacagtctccatttgtagctgaattcttgtc

SEQ ID NO: 607 - DNA, 60 bp, Artificial Sequence
  Synthetic oligonucleotide
gtctcgtgggctcggagatgtgtataagagacagtgtttttgtttttttaccactggctc

SEQ ID NO: 608 - DNA, 59 bp, Artificial Sequence
  Synthetic oligonucleotide
gtctcgtgggctcggagatgtgtataagagacaggccaaacagtgttttgtagaccatt

SEQ ID NO: 609 - DNA, 54 bp, Artificial Sequence
  Synthetic oligonucleotide
gtctcgtgggctcggagatgtgtataagagacaggatcagggggcagaaggatg

SEQ ID NO: 610 - DNA, 52 bp, Artificial Sequence
  Synthetic oligonucleotide
gtctcgtgggctcggagatgtgtataagagacagccctgctctgacaccagg

SEQ ID NO: 611 - DNA, 65 bp, Artificial Sequence
  Synthetic oligonucleotide
gtctcgtgggctcggagatgtgtataagagacagtgtttactaccaaaataatcaaaagcacaaa

SEQ ID NO: 612 - DNA, 51 bp, Artificial Sequence
  Synthetic oligonucleotide
gtctcgtgggctcggagatgtgtataagagacagtccttggcagccgttcc

SEQ ID NO: 613 - DNA, 54 bp, Artificial Sequence
  Synthetic oligonucleotide
gtctcgtgggctcggagatgtgtataagagacagaacacacagacaggcaggtt

SEQ ID NO: 614 - DNA, 65 bp, Artificial Sequence
  Synthetic oligonucleotide
gtctcgtgggctcggagatgtgtataagagacagagtgctcaatagttaccataatgctatattg

SEQ ID NO: 615 - DNA, 57 bp, Artificial Sequence
  Synthetic oligonucleotide
gtctcgtgggctcggagatgtgtataagagacagcctcctctctgtgtccatagaac

SEQ ID NO: 616 - DNA, 66 bp, Artificial Sequence
  Synthetic oligonucleotide
gtctcgtgggctcggagatgtgtataagagacagtcaatggttttaccatttaaaaattccctatc

SEQ ID NO: 617 - DNA, 57 bp, Artificial Sequence
  Synthetic oligonucleotide
gtctcgtgggctcggagatgtgtataagagacagctcagttgctcagaacaatgtcc

SEQ ID NO: 618 - DNA, 57 bp, Artificial Sequence
  Synthetic oligonucleotide
gtctcgtgggctcggagatgtgtataagagacagggagtttcatcaccaagtccaca

SEQ ID NO: 619 - DNA, 63 bp, Artificial Sequence
  Synthetic oligonucleotide
gtctcgtgggctcggagatgtgtataagagacagcccttgctatcaatattcaaagagagaaa

SEQ ID NO: 620 - DNA, 53 bp, Artificial Sequence
  Synthetic oligonucleotide
gtctcgtgggctcggagatgtgtataagagacaggctcgtaggtgtgcaccat

SEQ ID NO: 621 - DNA, 68 bp, Artificial Sequence
  Synthetic oligonucleotide
gtctcgtgggctcggagatgtgtataagagacagagaacattcaatgatataaaaggaataagagaac

SEQ ID NO: 622 - DNA, 53 bp, Artificial Sequence
  Synthetic oligonucleotide
gtctcgtgggctcggagatgtgtataagagacagtccttggagctgacatggc

SEQ ID NO: 623 - DNA, 56 bp, Artificial Sequence
  Synthetic oligonucleotide
gtctcgtgggctcggagatgtgtataagagacaggggatctctcatctcaggcttg

SEQ ID NO: 624 - DNA, 51 bp, Artificial Sequence
  Synthetic oligonucleotide
gtctcgtgggctcggagatgtgtataagagacagtgtgtgccctcgaaccg

SEQ ID NO: 625 - DNA, 54 bp, Artificial Sequence
  Synthetic oligonucleotide
gtctcgtgggctcggagatgtgtataagagacagcgaccccatctctgagtcct

SEQ ID NO: 626 - DNA, 57 bp, Artificial Sequence
  Synthetic oligonucleotide
gtctcgtgggctcggagatgtgtataagagacagctaggagcagtcaggcttaagag

SEQ ID NO: 627 - DNA, 56 bp, Artificial Sequence
  Synthetic oligonucleotide
gtctcgtgggctcggagatgtgtataagagacagtgaagaacatgcttgccatagc

SEQ ID NO: 628 - DNA, 55 bp, Artificial Sequence
  Synthetic oligonucleotide
gtctcgtgggctcggagatgtgtataagagacaggcactattcaggcaaaggctc

SEQ ID NO: 629 - DNA, 57 bp, Artificial Sequence
  Synthetic oligonucleotide
gtctcgtgggctcggagatgtgtataagagacagcccagaggattaagagacatggc

SEQ ID NO: 630 - DNA, 62 bp, Artificial Sequence
  Synthetic oligonucleotide
gtctcgtgggctcggagatgtgtataagagacagaagctctagaaaaggcaaaactaaacta

SEQ ID NO: 631 - DNA, 64 bp, Artificial Sequence
  Synthetic oligonucleotide
gtctcgtgggctcggagatgtgtataagagacagtcttcctattcagcctataagtgtttctaa

SEQ ID NO: 632 - DNA, 66 bp, Artificial Sequence
  Synthetic oligonucleotide
gtctcgtgggctcggagatgtgtataagagacagaaaacgacttacacatacctaaaatgaaattt

SEQ ID NO: 633 - DNA, 59 bp, Artificial Sequence
  Synthetic oligonucleotide
gtctcgtgggctcggagatgtgtataagagacagtttgatttgggagcaaagaatgagt

SEQ ID NO: 634 - DNA, 59 bp, Artificial Sequence
  Synthetic oligonucleotide
gtctcgtgggctcggagatgtgtataagagacaggcatctctatgccaaactggtcata

SEQ ID NO: 635 - DNA, 63 bp, Artificial Sequence
  Synthetic oligonucleotide
gtctcgtgggctcggagatgtgtataagagacagccaaactacttcttttctaacagaaagca

SEQ ID NO: 636 - DNA, 54 bp, Artificial Sequence
  Synthetic oligonucleotide
gtctcgtgggctcggagatgtgtataagagacaggcatctcttgtgtcagccct

SEQ ID NO: 637 - DNA, 59 bp, Artificial Sequence
  Synthetic oligonucleotide
gtctcgtgggctcggagatgtgtataagagacagccagggaaaaaatatgttcgatgcc

SEQ ID NO: 638 - DNA, 70 bp, Artificial Sequence
  Synthetic oligonucleotide
gtctcgtgggctcggagatgtgtataagagacaggataacatagtaatgaatacatttctaaaaccgtaa

SEQ ID NO: 639 - DNA, 65 bp, Artificial Sequence
  Synthetic oligonucleotide
gtctcgtgggctcggagatgtgtataagagacagtccagaagattagttgaaaatttgagtacaa

SEQ ID NO: 640 - DNA, 64 bp, Artificial Sequence
  Synthetic oligonucleotide
gtctcgtgggctcggagatgtgtataagagacagcgttttgtcatttttgcagataaatgtagt

SEQ ID NO: 641 - DNA, 53 bp, Artificial Sequence
  Synthetic oligonucleotide
gtctcgtgggctcggagatgtgtataagagacagagcaaaaccgcaacccact

SEQ ID NO: 642 - DNA, 62 bp, Artificial Sequence
  Synthetic oligonucleotide
gtctcgtgggctcggagatgtgtataagagacagactcatatctcccaacacaaaactaaaa

SEQ ID NO: 643 - DNA, 55 bp, Artificial Sequence
  Synthetic oligonucleotide
gtctcgtgggctcggagatgtgtataagagacagtgaagcgtgaacttcctcagg

SEQ ID NO: 644 - DNA, 54 bp, Artificial Sequence
  Synthetic oligonucleotide
gtctcgtgggctcggagatgtgtataagagacagcacctgggaagagttggtgt

SEQ ID NO: 645 - DNA, 59 bp, Artificial Sequence
  Synthetic oligonucleotide
gtctcgtgggctcggagatgtgtataagagacagtgaatatgtcttcagtgcttagcct

SEQ ID NO: 646 - DNA, 68 bp, Artificial Sequence
  Synthetic oligonucleotide
gtctcgtgggctcggagatgtgtataagagacagcctaaaaatcgttacttctcctttattttttttc

SEQ ID NO: 647 - DNA, 65 bp, Artificial Sequence
  Synthetic oligonucleotide
gtctcgtgggctcggagatgtgtataagagacaggcctataacaatgtactagaaccaagtattt

SEQ ID NO: 648 - DNA, 51 bp, Artificial Sequence
  Synthetic oligonucleotide
gtctcgtgggctcggagatgtgtataagagacaggggtgtagggcaggggt

SEQ ID NO: 649 - DNA, 58 bp, Artificial Sequence
  Synthetic oligonucleotide
gtctcgtgggctcggagatgtgtataagagacagttcacaagcagttgttgaaagact

SEQ ID NO: 650 - DNA, 63 bp, Artificial Sequence
  Synthetic oligonucleotide
gtctcgtgggctcggagatgtgtataagagacagaaaagactgtcagtgatatcttaggtaga

SEQ ID NO: 651 - DNA, 61 bp, Artificial Sequence
  Synthetic oligonucleotide
gtctcgtgggctcggagatgtgtataagagacagggccaacattttgttttataatctgcg

SEQ ID NO: 652 - DNA, 64 bp, Artificial Sequence
  Synthetic oligonucleotide
gtctcgtgggctcggagatgtgtataagagacagctaggatcaaagaagaatagaaaaagtggt

SEQ ID NO: 653 - DNA, 70 bp, Artificial Sequence
  Synthetic oligonucleotide
gtctcgtgggctcggagatgtgtataagagacagacaataattgtacttatttatggagtacatagtgat

SEQ ID NO: 654 - DNA, 59 bp, Artificial Sequence
  Synthetic oligonucleotide
gtctcgtgggctcggagatgtgtataagagacagcctaggaggtgttcctcactaaaat

SEQ ID NO: 655 - DNA, 60 bp, Artificial Sequence
  Synthetic oligonucleotide
gtctcgtgggctcggagatgtgtataagagacagactatctacatcagtgcgagagaaag

SEQ ID NO: 656 - DNA, 52 bp, Artificial Sequence
  Synthetic oligonucleotide
gtctcgtgggctcggagatgtgtataagagacagtgtgcagagtcccccagg

SEQ ID NO: 657 - DNA, 50 bp, Artificial Sequence
  Synthetic oligonucleotide
gtctcgtgggctcggagatgtgtataagagacagggcctccagcacgctc

SEQ ID NO: 658 - DNA, 54 bp, Artificial Sequence
  Synthetic oligonucleotide
gtctcgtgggctcggagatgtgtataagagacaggggtagattccaggggctct

SEQ ID NO: 659 - DNA, 60 bp, Artificial Sequence
  Synthetic oligonucleotide
gtctcgtgggctcggagatgtgtataagagacagggaacactatctgaaatagaccctcg

SEQ ID NO: 660 - DNA, 56 bp, Artificial Sequence
  Synthetic oligonucleotide
gtctcgtgggctcggagatgtgtataagagacagcctgcaggtctttccgattctg

SEQ ID NO: 661 - DNA, 66 bp, Artificial Sequence
  Synthetic oligonucleotide
gtctcgtgggctcggagatgtgtataagagacaggattttaaaaccagagataattctgaaaggaa

SEQ ID NO: 662 - DNA, 54 bp, Artificial Sequence
  Synthetic oligonucleotide
gtctcgtgggctcggagatgtgtataagagacaggctggtcctcactgacatcc

SEQ ID NO: 663 - DNA, 53 bp, Artificial Sequence
  Synthetic oligonucleotide
gtctcgtgggctcggagatgtgtataagagacagtctaaccccgtcatgctgc

SEQ ID NO: 664 - DNA, 67 bp, Artificial Sequence
  Synthetic oligonucleotide
gtctcgtgggctcggagatgtgtataagagacaggtgcaatgttaactttattaattagttgacttc

SEQ ID NO: 665 - DNA, 58 bp, Artificial Sequence
  Synthetic oligonucleotide
gtctcgtgggctcggagatgtgtataagagacagttgctgttccacaaaacatagctt

SEQ ID NO: 666 - DNA, 61 bp, Artificial Sequence
  Synthetic oligonucleotide
gtctcgtgggctcggagatgtgtataagagacaggctgattaattaggtgtttgttagcag

SEQ ID NO: 667 - DNA, 53 bp, Artificial Sequence
  Synthetic oligonucleotide
gtctcgtgggctcggagatgtgtataagagacagcctcaccctccatccctca

SEQ ID NO: 668 - DNA, 50 bp, Artificial Sequence
  Synthetic oligonucleotide
gtctcgtgggctcggagatgtgtataagagacagcccaaggcgggcacct

SEQ ID NO: 669 - DNA, 50 bp, Artificial Sequence
  Synthetic oligonucleotide
gtctcgtgggctcggagatgtgtataagagacaggggtgctgcgcccaga

SEQ ID NO: 670 - DNA, 67 bp, Artificial Sequence
  Synthetic oligonucleotide
gtctcgtgggctcggagatgtgtataagagacagggataaactagtggttctttgatctttatcttt

SEQ ID NO: 671 - DNA, 65 bp, Artificial Sequence
  Synthetic oligonucleotide
gtctcgtgggctcggagatgtgtataagagacagaacatattgaacttatttttaaaagggggag

SEQ ID NO: 672 - DNA, 53 bp, Artificial Sequence
  Synthetic oligonucleotide
gtctcgtgggctcggagatgtgtataagagacaggggggatctgccatacagc

SEQ ID NO: 673 - DNA, 58 bp, Artificial Sequence
  Synthetic oligonucleotide
gtctcgtgggctcggagatgtgtataagagacagtcaagccttgacttttaaagcaca

SEQ ID NO: 674 - DNA, 67 bp, Artificial Sequence
  Synthetic oligonucleotide
gtctcgtgggctcggagatgtgtataagagacagttgttgttgtcagcttagaatagtatatcatat

SEQ ID NO: 675 - DNA, 68 bp, Artificial Sequence
  Synthetic oligonucleotide
gtctcgtgggctcggagatgtgtataagagacagcccttttataatcttgttacttttatgttgaact

SEQ ID NO: 676 - DNA, 57 bp, Artificial Sequence
  Synthetic oligonucleotide
gtctcgtgggctcggagatgtgtataagagacagggggagagaaaaccgtatgtgat

SEQ ID NO: 677 - DNA, 62 bp, Artificial Sequence
  Synthetic oligonucleotide
gtctcgtgggctcggagatgtgtataagagacagttttagatctctttcagttttggtttgc

SEQ ID NO: 678 - DNA, 65 bp, Artificial Sequence
  Synthetic oligonucleotide
gtctcgtgggctcggagatgtgtataagagacagggagttataaaaaagaacagaaggtcatgat

SEQ ID NO: 679 - DNA, 52 bp, Artificial Sequence
  Synthetic oligonucleotide
gtctcgtgggctcggagatgtgtataagagacaggcctccttcactcgcagt

SEQ ID NO: 680 - DNA, 52 bp, Artificial Sequence
  Synthetic oligonucleotide
gtctcgtgggctcggagatgtgtataagagacaggcacaagcggtcaacagc

SEQ ID NO: 681 - DNA, 57 bp, Artificial Sequence
  Synthetic oligonucleotide
gtctcgtgggctcggagatgtgtataagagacaggggtttatcaaaaggtttgctgc

SEQ ID NO: 682 - DNA, 62 bp, Artificial Sequence
  Synthetic oligonucleotide
gtctcgtgggctcggagatgtgtataagagacagcgctaaaaaaggaagaactaggaaagat

SEQ ID NO: 683 - DNA, 70 bp, Artificial Sequence
  Synthetic oligonucleotide
gtctcgtgggctcggagatgtgtataagagacagaatatctatccagagtatcgattaatcttcataaaa

SEQ ID NO: 684 - DNA, 65 bp, Artificial Sequence
  Synthetic oligonucleotide
gtctcgtgggctcggagatgtgtataagagacagttcctagtacgtcatttataatgaaaattgc

SEQ ID NO: 685 - DNA, 78 bp, Artificial Sequence
  Synthetic oligonucleotide
cagacataaacagaagctttagtagattatgctggtattacggtggaaggcaaggcaagatacagacaaaaatcaaaa

SEQ ID NO: 686 - DNA, 78 bp, Artificial Sequence
  Synthetic oligonucleotide
cagacataaacagaagctttagtagattatgctggtattagggtggaaggcaaggcaagatacagacaaaaatcaaaa

SEQ ID NO: 687 - DNA, 21 bp, Artificial Sequence
  Synthetic oligonucleotide
aaggcagagcaaatgtacagg

(19) United States
(12) Patent Application Publication
(10) Pub. No.: US 2022/0267848 A1
(43) Pub. Date: Aug. 25, 2022

(54) DETECTION AND QUANTIFICATION OF RARE VARIANTS WITH LOW-DEPTH SEQUENCING VIA SELECTIVE ALLELE ENRICHMENT OR DEPLETION

(71) Applicant: William Marsh Rice University, Houston, TX (US)

(72) Inventors: David ZHANG, Houston, TX (US); Juexiao WANG, Houston, TX (US)

(73) Assignee: William Marsh Rice University, Houston, TX (US)

(21) Appl. No.: 17/735,062

(22) Filed: May 2, 2022

Related U.S. Application Data
(63) Continuation of application No. 16/227,790, filed on Dec. 20, 2018, now abandoned.
(60) Provisional application No. 62/608,197, filed on Dec. 20, 2017.

(51) Int. Cl. C12Q 1/6876; C12Q 1/6869; C12Q 1/6886

(52) U.S. Cl. CPC C12Q 1/6876; C12Q 1/6869; C12Q 1/6886; C12Q 2600/156; C12Q 2600/16

(57) ABSTRACT

This disclosure describes methods for enabling accurate detection and quantitation of rare alleles within a DNA sample using low-depth sequencing, through the use of allele-specific enrichment and/or depletion hybridization probes. For example, methods are provided for using competitive probes to apply allele-specific enrichment or depletion to amplicons from multiplex PCR on a biological DNA sample.

### REFERENCE TO RELATED APPLICATIONS

The present application is a continuation of U.S. application Ser. No. 16/227,790, filed Dec. 20, 2018, which claims the priority benefit of U.S. Provisional Application No. 62/608,197, filed Dec. 20, 2017, the entire contents of each of which are incorporated herein by reference.

### STATEMENT REGARDING FEDERALLY SPONSORED RESEARCH

This invention was made with government support under Grant No. R01 HG008752 awarded by the National Institutes of Health. The government has certain rights in the invention.

### BACKGROUND

### 1. Field

The present disclosure relates generally to the field of molecular biology. More particularly, it concerns methods of enhancing detection of sequence variants by selective allele enrichment or depletion prior to sequencing by next-generation sequencing.

### 2. Description of Related Art

In biological samples, such as cell-free DNA from peripheral blood, rare DNA sequence variants, such as cancer driver mutations, are present at less than 1% allele frequency, but can nonetheless provide important therapy guidance or patient stratification information. Additionally, there is a need to simultaneously analyze many potential mutations to achieve high clinical sensitivity. Next-generation sequencing has been applied to detection and quantitation of rare DNA variants through deep sequencing with molecular barcodes. However, these methods are inherently inefficient and expensive due to the large number of NGS reads wasted on sequencing wildtype (i.e., healthy) DNA.

### SUMMARY

The disclosure describes a class of methods to allow low-throughput detection and quantification of rare variants, such as somatic cancer mutations in peripheral blood plasma. The large number of reads needed for liquid biopsy applications prevents the detection and quantification of rare events by low-throughput NGS. However, allelic enrichment/depletion enables low-throughput NGS instruments, such as the Illumina MiSeq, the Qiagen GeneReader, and the Thermo Fisher Proton systems, to perform liquid biopsy detection of cancer mutations (see FIG. 6).

In one embodiment, provided herein are methods of detecting the presence of rare sequence variants within a DNA region of interest, the method comprising: (a) amplifying one or more region of interest using polymerase chain reaction (PCR) with primers, each primer comprising a 5′ sequence-adaptor region and a 3′ gene-specific region, thereby generating double-stranded amplicons; (b) denaturing the double-stranded amplicons, thereby generating single-stranded amplicons; (c) hybridizing the single-stranded amplicons to a mixture of negative-selection Sinks; (d) removing the single-stranded amplicons bound to Sinks; (e) amplifying the remaining single-stranded amplicons by PCR using primers comprising sequencing adaptor sequences; and (f) performing high-throughput DNA sequencing. In some aspects, the rare variant is of unknown sequence identity. In some aspects, the rare variant is of known sequence identity.

In some aspects, step (c) further comprises hybridizing the single-stranded amplicons to a mixture of positive-selection Probes. In certain aspects, the Probes comprise toehold probes, fine-tuned probes, or X-probes. In certain aspects, the Probes and Sinks are thermodynamically competitive. In some aspects, there is one Probe and one Sink for each rare sequence variant. In some aspects, there is one Probe for each rare sequence variant. In some aspects, two or more rare sequence variants may use the same Sink. In some aspects, the Probes comprise Probes having paired probe complement and probe protector oligonucleotides of Table 1. In some aspects, the Sink comprise Sinks having paired sink complement and sink protector oligonucleotides of Table 2. In certain aspects, step (d) further comprises collecting amplicons bound to Probes. In certain aspects, step (d) is performed via streptavidin-coated magnetic beads, collecting is performed using a magnet, and the Probes in step (c) are either directly functionalized with a biotin or hybridized to a universal oligonucleotide functionalized with a biotin. In certain aspects, step (d) is performed via streptavidin-coated agarose beads, collection is performed using centrifugal force, and the Probes in step (c) are either directly functionalized with a biotin or hybridized to a universal oligonucleotide functionalized with a biotin. In certain aspects, removing the single-stranded amplicons bound to Sinks occurs by way of collecting amplicons bound to Probes.

In some aspects, the hybridization in step (c) is performed at a temperature of between about 15° C. and about 75° C. In some aspects, the hybridization in step (c) is performed in a buffer with a monovalent cation concentration of between about 50 mM and about 5 M. In certain aspects, the monovalent cation is sodium. In some aspects, the hybridization in step (c) is performed in a buffer with a divalent cation concentration of between about 3 mM and about 30 mM. In certain aspects, the divalent cation is magnesium.

In some aspects, the PCR of step (a) is multiplex PCR when amplifying more than one region of interest. In some aspects, the PCR of step (a) is carried out for 4-20 cycles. In some aspects, the PCR of step (a) is carried out for no more than 4, 5, 6, 7, 8, 9, 10, 11, 12, 13, 14, 15, 16, 17, 18, 19, or 20 cycles.

In some aspects, step (b) is performed via heat denaturation. In certain aspects, heat denaturation comprises heating the amplicon mixture to at least 80° C. for at least 2 minutes. In some aspects, step (b) is performed via DNAse activity and wherein one of the primers in step (a) is modified with either a 5′ phosphate functionalization to encourage degradation or a 5′ functionalization to inhibit degradation. In certain aspects, the 5′ primer functionalization comprises a phosphorothioate, a 2′-O-methyl group, or a non-natural nucleotide.

In some aspects, the Sinks in step (c) comprise toehold probes, fine-tuned probes, or X-probes. In some aspects, the removing in step (d) is performed via solid-phase separation. In some aspects, step (d) is performed via streptavidin-coated magnetic beads, removing is performed using a magnet, and the Sinks in step (c) are either directly functionalized with a biotin or hybridized to a universal oligonucleotide functionalized with a biotin. In some aspects, step (d) is performed via streptavidin-coated agarose beads, removing is performed using centrifugal force, and the Sinks in step (c) are either directly functionalized with a biotin or hybridized to a universal oligonucleotide functionalized with a biotin.

In some aspects, the primers in step (e) are universal primers. In some aspects, the primers in step (e) further comprise a sample barcode or index sequence. In some aspects, the sequencing in step (f) is sequencing-by-synthesis. In some aspects, the sequencing in step (f) is nanopore sequencing. In some aspects, the sequencing in step (f) is sequencing-by-hybridization (e.g., Nanostring).

In some aspects, the method further comprises (g) analyzing the DNA sequencing data to calculate the ratio of reads observed for variant sequences as compared to wild-type sequences. In some aspects, the sequencing in step (f) is paired-end sequencing. In some aspects, the analysis in step (g) does not consider any sequencing read in which the forward read and the reverse read do not perfectly agree on the sequence of the amplicon insert. In certain aspects, the analysis in step (g) does not consider any sequencing reading in which a read quality score is below 30. In certain aspects, the read quality score is a threshold FASTQ score.

In some aspects, the method is further defined as a method of quantifying the presence of rare sequence variants within a DNA region of interest.

As used herein, “essentially free,” in terms of a specified component, means that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1: Example experimental workflow for enriching suspected genomic variants using positive-selection Probes and negative-selection Sinks. The concentration of gene-specific primers introduced in step 1 range between 5 and 80 nM. In step 2, the PCR protocol consists of an initial 98° C. denaturation for 10 minutes, followed by 5 cycles of (98° C. for 20 sec, 62° C. for 4 min, and 72° C. for 1 min) using the KAPA High Fidelity DNA polymerase. In step 4, the variant-specific Probes and wildtype-specific Sinks are introduced; the Probes are pre-hybridized to a universal biotinylated oligo whereas the Sinks are not. Both the Probes and the Sinks are double-stranded “toehold” or “fine-tuned” probes that each includes an auxiliary “Protector” oligonucleotide, which hybridizes to the Probe's complement strand. The stoichiometry of the Probe's protector varies between 1.5× and 9× relative to the Probe's complement strand. The stoichiometry of the Sink's protector varies between 2× and 11× relative to the Sink's complement strand. In step 7, the number of PCR cycles varies between 22 and 27 depending on sample input quantity and variant allele frequency, as well as prior expectations on the panel's fold enrichment. This PCR protocol consists of an initial 98° C. denaturation for 10 minutes, followed by 5 cycles of (98° C. for 20 sec, 62° C. for 15 sec, and 72° C. for 30 sec) using the KAPA High Fidelity DNA polymerase.

FIG. 2: Example of the computational workflow for analyzing sequencing results. This workflow is conservative in the sense that reads with any indication of error will be discarded, and only perfect reads will be used for assessing the allelic fraction of the Variant. Conservative analysis workflows tend to produce higher confidence on the allelic fraction at the cost of lower fraction of usable reads and sequencing depth.

FIGS. 3A-B: Experimental results for a 114-plex Variant enrichment panel using both positive and negative selection. The genomic DNA input sample consisted of 498.5 ng NA18537 cell line DNA and 1.5 ng NA18562 cell line DNA. The sample is thus 0.3% allele frequency in all single nucleotide polymorphisms (SNPs) in which both NA18537 and NA18562 are homozygous but differ from each other. The FIG. 3A graph shows that in a 2.2M read library, with roughly 10,000× depth per locus, there are roughly 30 variant reads per locus, as expected for the 0.3% allele frequency sample. For enrichment, Probes were designed to NA18562 SNP alleles and Sinks were designed to NA18537 alleles. The FIG. 3B graph shows that a 63 k read library produces similar reads for the variant for each locus, while the sequencing depth has been reduced 36-fold. Thus, sequencing cost can be reduced 36-fold while attaining similar information on rare mutations. See Tables 1-3 for sequences of the primers and probe oligonucleotides used to generate these data.

FIG. 4: Distribution of fold-enrichment per locus for the 114-plex panel summarized in FIG. 3. Median fold-enrichment observed was 52, and 90% of the Variants were enriched 8-fold or more. Fold-enrichment can be improved through empirical optimization of Probe or Sink sequence, or of Probe protector and Sink protector stoichiometry.

FIG. 5: Sequences for the Variant (SEQ ID NO: 685), Wild-type (SEQ ID NO: 686), Probe (SEQ ID NO: 6), Probe Protector (SEQ ID NO: 120), Universal Oligonucleotide Functionalized with Biotin (SEQ ID NO: 687), Sink (SEQ ID NO: 234), and Sink Protector (SEQ ID NO: 348) for one locus in the 114-plex panel. See Tables 1-3 for the full sequence list used for the 114-plex panel.

FIGS. 6A-B: Allele-selective enrichment sequencing (ASES). (FIG. 6A) Profiling rare mutations in cell-free DNA (cfDNA) requires extremely high sequencing depth as well as unique molecular identifier (UMI) barcodes. (FIG. 6B) ASES uses highly sequence-selective hybridization probes to enrich the variant allele fraction, allowing rare mutation profiling using low-depth sequencing.

FIGS. 7A-E: ASES sources of error and VAF limit of detection. (FIG. 7A) False positives arise from either PCR errors due to limited enzyme fidelity (e1) or NGS sequencing error (e0). (FIG. 7B) When using a pure human gDNA, all cancer mutations in the panel should have a VAF of 0%; non-zero VRF thus corresponds to the false positive error rate. (FIG. 7D) Overall distribution of ASES error rate in (FIG. 7C). (FIG. 7E) Per-locus error rate comparison between ASES and deep-sequencing. Error bar shows the standard deviation of the mean. Traces were ranked by mean per-locus error rate of ASES.

FIGS. 8A-D: Demonstration of ASES on an 118-plex non-pathogenic SNP panel. (FIG. 8A) Library preparation workflow. (FIG. 8B) NGS reads mapped to variant SNP alleles vs. wildtype alleles using standard deep sequencing. (FIG. 8C) NGS reads mapped to the variant SNP alleles vs. wildtype alleles using ASES. (FIG. 8D) Distribution of variant read fraction (VRF, variant reads divided by total reads mapped to each SNP locus) for standard deep sequencing vs. ASES.

FIGS. 9A-G: Estimation of sample VAF based on ASES VRF. (FIG. 9A) Consistent and predictable relationship between VRF, VAF, and fold-enrichment E. The dots represent experimental results from 7 different NGS libraries, where the input sample had known VAFs of 0.1%, 0.2%, 0.3%, 0.5%, 0.7%, 1%, 2%. The solid lines show theoretical curves for different values of E best-fitted to the three shown SNPs. The right panel plots VAF and VRF under non-linear transformations. (FIG. 9B) Distribution of r² for the 118 SNP loci in the non-pathogenic ASES panel. One outlier SNP locus with r2<0 was omitted from the plot. (FIG. 9C) Distribution of best-t E. Error bars show the root mean square error (RMSE) of the linear t to the 7 data points for each SNP. Asterisk represents the one SNP in which the 0.1% VAF library did not have enough reads in the SNP locus to allow VRF quantitation; that SNP fitted E using the other 6 data points. (FIG. 9D) Distribution of fitted E for different base substitution types. One-way ANOVA indicates a p-value of 0.21, indicating that there is likely little to no sequence-based bias in E. (FIG. 9E) Accuracy of inferred VAF based on observed VRF and fitted E. (FIG. 9F) VAF quantitation based on VRF for standard deep sequencing. (FIG. 9G) NGS read uniformity across the 118 different amplicons, visualized by the cumulative distribution plots of NGS reads vs. number of loci (Lorenz curve).

FIGS. 10A-E: ASES actionable cancer mutation panel. (FIG. 10A) The distribution of mutations by corresponding cancer type, and the number of mutations profiled on each gene. (FIG. 10B) Distribution of potential cancer mutations on amplicons. (FIG. 10C) NGS reads mapped to cancer mutations vs. wildtype using standard deep sequencing (left) and ASES (right). (FIG. 10D) Distribution of observed fold-enrichment E, both in aggregate (left) and sorted by colocalization type (right). (FIG. 10E) Validation of cancer mutation panel on Horizon cfDNA reference samples (HD780 Multiplex I).

FIGS. 11A-B: Validation of the ASES cancer mutation panel on clinical cfDNA samples. (FIG. 11A) Summary of called mutations in 6 samples by deep sequencing, and in 64 samples by ASES. (FIG. 11B) Side-by-side comparison of ASES and deep sequencing on equal size aliquots of the same clinical cfDNA samples.

### DESCRIPTION OF ILLUSTRATIVE EMBODIMENTS

The present disclosure describes methods for using toehold probes, fine-tune probes, or X-probes to apply allele-specific enrichment or depletion to amplicons from multiplex PCR on a biological DNA sample. Due to the high sequence specificity of the probes, a large majority of the wild-type sequences are removed, and the allele frequency of mutations are significantly increased. Consequently, low-depth sequencing becomes sufficient to detect and quantitate rare mutations. Thus, the industrial applicability of this disclosure is to significantly reduce sequencing costs for analyzing rare mutations.

Although these allele-specific enrichment and depletion probes have been previously demonstrated on DNA targets, integration with NGS is non-trivial. For example, direct application of toehold probes to biological DNA is undesirable because low probe capture yield and limited sample input quantity may result in false negatives, in which rare mutations are present in the original DNA sample but not captured by probes and consequently not represented in NGS data.

Furthermore, the current dominant method of NGS analysis of biological DNA is to end-repair fragmented genomic DNA and subsequently ligate to sequencing adaptors. However, end-repair and ligation are both low-yield enzymatic processes, and likewise can result in false negatives due to losing the few DNA molecules that bear a rare mutation.

Yet another possible but ultimately undesirable method is to perform many cycles of multiplexed amplification of gene regions of interest, and perform toehold probe enrichment or depletion on the final product. The drawback of this approach is that with high cycle multiplexed PCR, primer dimers become dominant, practically preventing this approach from scaling to more than 20 genetic loci.

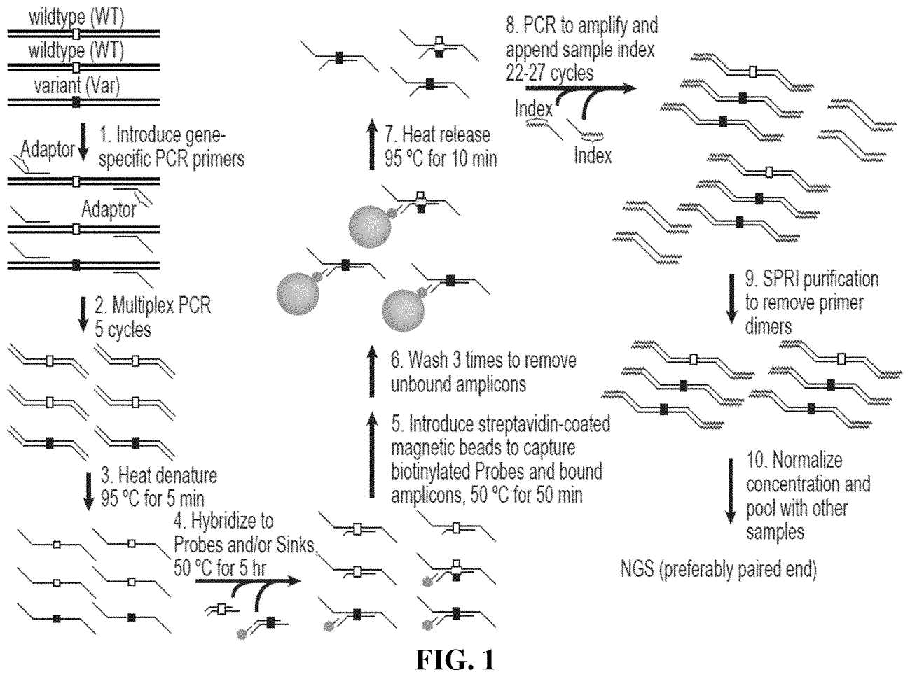

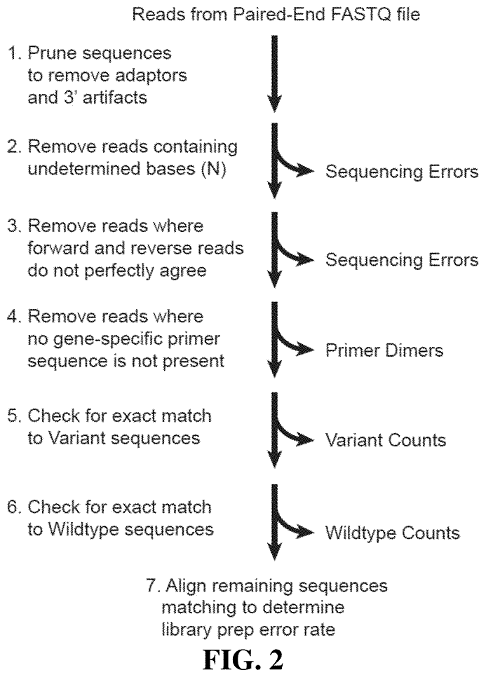

The present disclosure thus describes the approach of performing low-cycle (e.g., 5) multiplex PCR to pre-amplify gene regions of interest by roughly 10- to 30-fold to counter probe binding loss, while simultaneously remaining scalable to high multiplexing due to the unlikelihood of accumulating high concentrations of primer dimers within only a few PCR cycles (see FIG. 1).

In some embodiments, both positive selection of the known rare variant is performed in combination with negative selection of the corresponding wild-type allele. In other embodiments, negative selection of wild-type alleles may be formed without concurrent positive selection, which allows for the detection of rare variants of unknown sequence.

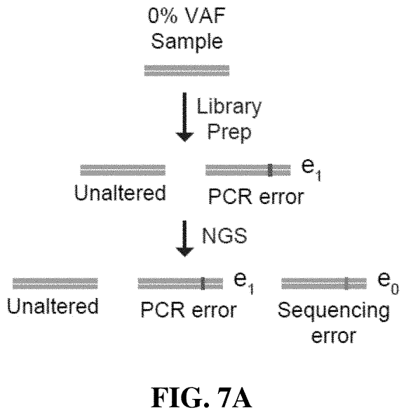

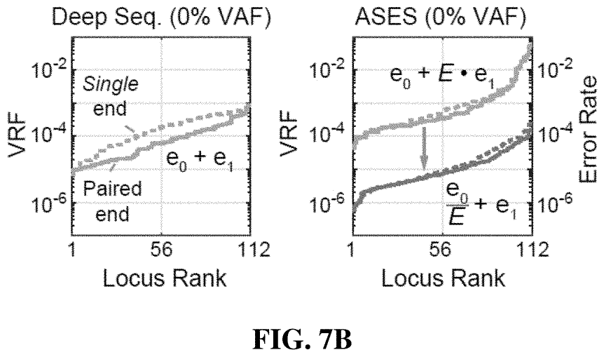

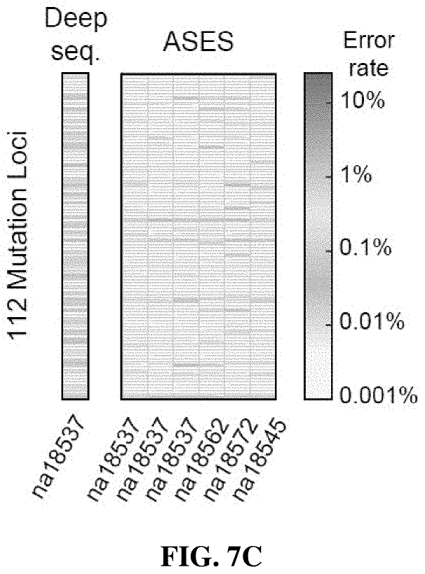

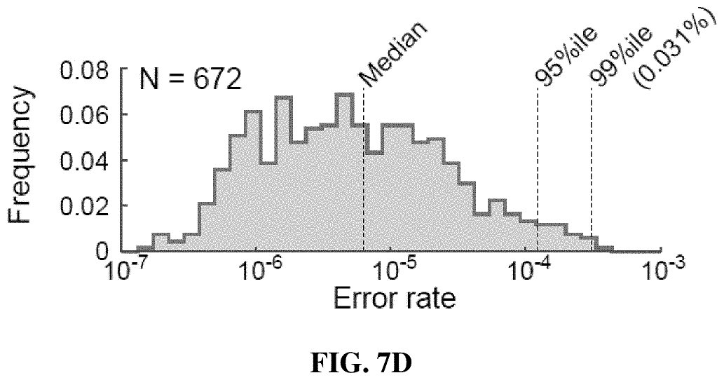

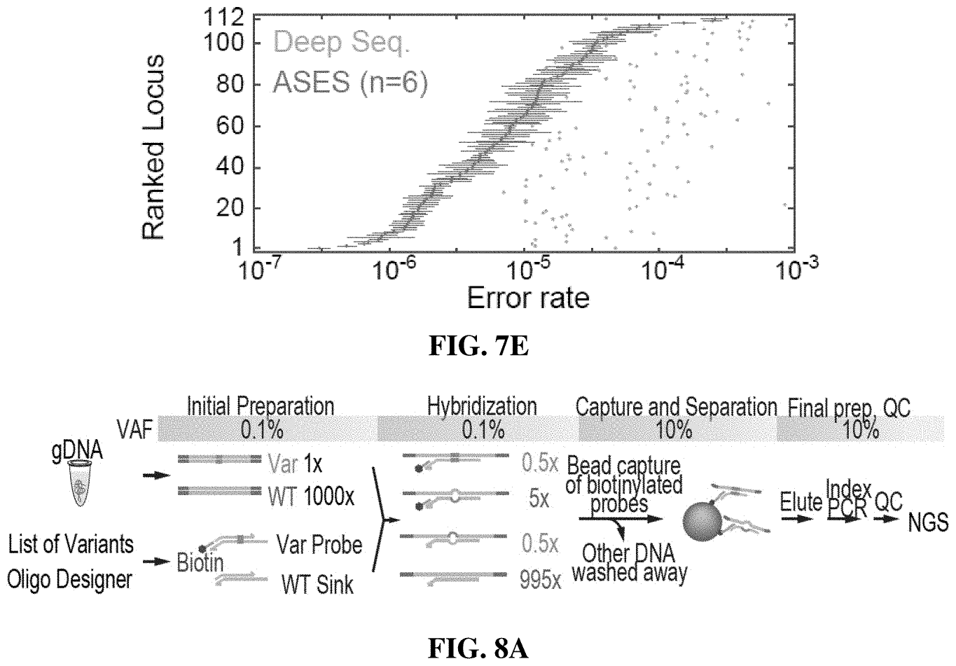

ASES sources of error and VAF limit of detection. False positives arise from either PCR errors due to limited enzyme fidelity (e1) or NGS sequencing error (e0) (FIG. 7A). For example, when using a pure human gDNA, all cancer mutations in the panel should have a VAF of 0%; non-zero VRF thus corresponds to the false positive error rate (FIG. 7B). Median error rate of ASES was 0.610□5, representing an almost 10-fold reduction in errors as compared to deep sequencing (FIGS. 7C-D). A per-locus error rate comparison between ASES and deep-sequencing is shown in FIG. 7E.

### I. NUCLEIC ACID PROBES

In some embodiments, the present disclosure provides synthetic oligonucleotide probes for use in allele-specific enrichment and/or depletion. In particular embodiments, the oligonucleotide probes are toehold probes, X-probes, or fine-tune probes. The oligonucleotide probes can have a length of 30 to 200 nucleotides, particularly 50 to 100 nucleotides, such as between 60 and 70 nucleotides. Further, the oligonucleotide probes can comprise part or all of sequencing primer sequences or their binding sites, such as index sequencing primers for particular sequencing platforms (e.g., Illumina index primers).

The molecular specificity of the enrichment and depletion probes is beneficial to the accurate inference of genomic DNA variants. Nonspecific binding of variant enrichment probes to wild-type loci would defeat the purpose of enrichment. Likewise, nonspecific binding of wild-type depletion probes to variant loci would result in the desired target being lost from the sample. Toehold probes with protector oligonucleotides can be employed to enhance the molecular specificity of the Probes and Sinks. In some aspects, the toehold probes may be fine-tune probes as described in U.S. Pat. Publn. No. 2016/0340727, which is incorporated herein by reference in its entirety. In some aspects, the toehold probes may be X-probes as described in U.S. Pat. Publn. No. 2016/0326600, which is incorporated herein by reference in its entirety.

In some embodiments, a protector oligonucleotide comprising a region that is partially complementary to the target complementarity region is introduced. Importantly, at least five continuous nucleotides on the target complementarity region are not bound by the protector, i.e., form a toehold, in order to allow initiation of hybridization between the target and the Probe/Sink. This protector oligonucleotide can improve the specificity of hybridization reactions (see Zhang et al., 2012, Wang and Zhang, 2015, U.S. Pat. No. 9,284,602, and U.S. Pat. Publn. No. 2016/0340727, each of which is incorporated herein by reference in its entirety), and maintains high sequence selectivity across a large range of temperatures and buffer conditions. In some aspects, the protector oligonucleotide is present in molar excess.

In some embodiments, the nucleic acid probes are rationally designed so that the standard free energy for hybridization (e.g., theoretical standard free energy) between the specific target nucleic acid molecule and the target complementarity region is close to zero, while the standard free energy for hybridization between a spurious target (even one differing from the specific (actual) target by as little as a single nucleotide) and the probe is high enough to make their binding unfavorable by comparison.

The “toehold” region is present in the target complementarity region, is complementary to a target sequence and not complementary to the protector oligonucleotide. The sequences of the complementary regions are rationally designed to achieve this matching under desired conditions of temperature and probe concentration. As a result, the equilibrium for the actual target and Probe/Sink rapidly approaches 50% target:probe::protector:probe (or whatever ratio is desired), while equilibrium for the spurious target and primer greatly favors protector:probe.

Mechanistically, it is thought that hybridization to a target begins at the toehold and continues along the length of the target complementarity region until the probe is no longer “double-stranded.” This assumes complementarity between the target and the target complementarity region. When nucleotide mismatches exist between a spurious target and the target complementarity region, displacement of the second strand (i.e., the protector oligonucleotide) is thermodynamically unfavorable and the association between the target complementarity region and the spurious target is reversed.

Because the standard free energy favors a complete match (fully complementary) between the target sequence of the nucleic acid and toehold regions of the probe rather than a mismatch (e.g., single nucleotide change), the target complementarity region of the probe will bind stably to a target in the absence of a mismatch but not in the presence of a mismatch. If a mismatch exists between the target complementarity region of the probe and the target, the probe duplex prefers to reform. In this way, the frequency of producing a ligation product when a target sequence is not present is reduced. This type of discrimination is typically not possible using the standard single-stranded probes because in those reactions there is no competing nucleic acid strand (such as the protector oligonucleotide) to which a mismatched probe strand would prefer to bind. In some aspects, the thermodynamics of the Probes and Sinks are designed to satisfy that of a Competitive Composition. See, U.S. Pat. Publn. No. US2017/0029875, which is incorporated herein by reference in its entirety.

In some aspects, the Sinks are functionalized with, for example, a biotin group to enable the removal of any target nucleic acids that are bound by the Sinks. In other aspects, the Probes are functionalized but the Sinks are not, thereby allowing any target nucleic acids bound by the Probes to be collected; in this aspect, the Sinks serve to compete with the Probes for binding to the non-desired targets to increase the specificity of the hybridization of the Probes.

In some embodiments, the sequence of the functionalized strand is decoupled from the sequence of the target-specific strand, such as, for example, in the case of X-probes. See, e.g., U.S. Pat. Publn. No. 2016/0326600, which is incorporated by reference herein in its entirety. In this embodiment, the probe system comprises a universal component and a target-specific component. The universal component comprises at least a first universal oligonucleotide/strand, which comprises at least one region. The sequence of the universal strand is not target specific and therefore can be used with any target-specific component. The target-specific component comprises a protector strand and a target-specific/complement strand. The target-specific strand (i.e. complement strand) comprises at least two regions. At least one of the regions of the target-specific strand is fully or partially complementary to the at least one region of the first universal strand, which gives rise to a first double-stranded region. In some instances, the protector strand has a region that is at least partially complementary (and in some instances fully complementary) to all or a portion of the target-specific strand, which gives rise to a second double-stranded region. The target-specific strand contains a toehold region that is not hybridized to any other strand of the probe, but is complementary to a portion of the target sequence. To be clear, the region of the target-specific strand that is complementary to a region of the protector strand is also complementary to the target sequence. In some embodiments, the first universal strand comprises a functionalization conjugated thereto.

Upon hybridization of the probe to the target nucleic acid, the protector strand and any universal strand hybridized thereto dissociates from the target-specific strand leaving the target-specific strand, along with any universal strand hybridized thereto, hybridized to the target nucleic acid. Thus, the probes of the present disclosure permit the use of functionalized universal components with a variety of target-specific components, thereby eliminating the expense of synthesizing a different functionalized probe for each desired target sequence.

In some aspects, the universal strands on the Sinks are functionalized with, for example, a biotin group to enable the removal of any target nucleic acids that are bound by the Sinks. In other aspects, the universal strands on the Probes are functionalized but the universal strands on the Sinks are not, thereby allowing any target nucleic acids bound by the Probes to be collected; in this aspect, the Sinks serve to compete with the Probes for binding to the non-desired targets to increase the specificity of the hybridization of the Probes.

### II. FURTHER PROCESSING OF TARGET NUCLEIC ACIDS

A. Target Nucleic Acid Molecules

A nucleic acid molecule of interest can be a single nucleic acid molecule or a plurality of nucleic acid molecules. Also, a nucleic acid molecule of interest can be of biological or synthetic origin. Examples of nucleic acid molecules include genomic DNA, cDNA, RNA, amplified DNA, a pre-existing nucleic acid library, etc.

Nucleic acids in a nucleic acid sample being analyzed (or processed) in accordance with the present disclosure can be from any nucleic acid source. As such, nucleic acids in a nucleic acid sample can be from virtually any nucleic acid source, including but not limited to genomic DNA, complementary DNA (cDNA), RNA (e.g., messenger RNA, ribosomal RNA, short interfering RNA, microRNA, etc.), plasmid DNA, mitochondrial DNA, etc. Furthermore, as any organism can be used as a source of nucleic acids to be processed in accordance with the present disclosure, no limitation in that regard is intended. Exemplary organisms include, but are not limited to, plants, animals (e.g., reptiles, mammals, insects, worms, fish, etc.), bacteria, fungi (e.g., yeast), viruses, etc. In certain embodiments, the nucleic acids in the nucleic acid sample are derived from a mammal, where in certain embodiments the mammal is a human. A nucleic acid molecule of interest can be a single nucleic acid molecule or a plurality of nucleic acid molecules. Also, a nucleic acid molecule of interest can be of biological or synthetic origin. Examples of nucleic acid molecules include genomic DNA, cDNA, cell-free DNA (cfDNA), RNA, amplified DNA, a pre-existing nucleic acid library, etc. In some aspects, the target nucleic acid is a double-stranded DNA molecule, such as, for example, human genomic DNA.

A nucleic acid molecule of interest may be subjected to various treatments, such as repair treatments and fragmenting treatments. Fragmenting treatments include mechanical, sonic, chemical, enzymatic, degradation over time, etc. Repair treatments include nick repair via extension and/or ligation, polishing to create blunt ends, removal of damaged bases such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

An RNA molecule may be obtained from a sample, such as a sample comprising total cellular RNA, a transcriptome, or both; the sample may be obtained from one or more viruses; from one or more bacteria; or from a mixture of animal cells, bacteria, and/or viruses, for example. The sample may comprise mRNA, such as mRNA that is obtained by affinity capture. Obtaining nucleic acid molecules may comprise generation of the cDNA molecule by reverse transcribing the mRNA molecule with a reverse transcriptase, such as, for example Tth DNA polymerase, HIV Reverse Transcriptase, AMV Reverse Transcriptase, MMLV Reverse Transcriptase, or a mixture thereof.

B. Amplification of Nucleic Acids

A number of template-dependent processes are available to amplify the target nucleic acids present in a given sample. One of the best known amplification methods is the polymerase chain reaction (referred to as PCR™) which is described in detail in U.S. Pat. Nos. 4,683,195, 4,683,202, and 4,800,159, each of which is incorporated herein by reference in its entirety. Briefly, two synthetic oligonucleotide primers, which are complementary to two regions of the template DNA (one for each strand) to be amplified, are added to the template DNA (that need not be pure), in the presence of excess deoxynucleotides (dNTP's) and a thermostable polymerase, such as, for example, Taq (Thermus aquaticus) DNA polymerase. In a series (typically 30-35) of temperature cycles, the target DNA is repeatedly denatured (around 90° C.), annealed to the primers (typically at 50-60° C.) and a daughter strand extended from the primers (72° C.). As the daughter strands are created they act as templates in subsequent cycles. Thus, the template region between the two primers is amplified exponentially, rather than linearly.

A barcode, such as a sample barcode, may be added to the target nucleic acid molecules during amplification. One method involves annealing a primer to the target nucleic acid molecule, the primer including a first portion complementary to the target nucleic acid molecule and a second portion including a barcode; and extending the annealed primer to form a barcoded nucleic acid molecule. Thus, the primer may include a 3′ portion and a 5′ portion, where the 3′ portion may anneal to a portion of the target nucleic acid molecule and the 5′ portion comprises the barcode.

C. Sequencing of Nucleic Acids

Methods are also provided for the sequencing of the library of nucleic acid molecules. Any technique for sequencing nucleic acids known to those skilled in the art can be used in the methods of the present disclosure. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD sequencing.

The nucleic acid library may be generated with an approach compatible with Illumina sequencing such as a Nextera™ DNA sample prep kit, and additional approaches for generating Illumina next-generation sequencing library preparation are described, e.g., in Oyola et al. (2012). In other embodiments, a nucleic acid library is generated with a method compatible with a SOLiD™ or Ion Torrent sequencing method (e.g., a SOLiD® Fragment Library Construction Kit, a SOLiD® Mate-Paired Library Construction Kit, SOLiD® ChIP-Seq Kit, a SOLiD® Total RNA-Seq Kit, a SOLiD® SAGE™ Kit, a Ambion® RNA-Seq Library Construction Kit, etc.). Additional methods for next-generation sequencing methods, including various methods for library construction that may be used with embodiments of the present disclosure are described, e.g., in Pareek (2011) and Thudi (2012).

In particular aspects, the sequencing technologies used in the methods of the present disclosure include the HiSeq™ system (e.g., HiSeq™ 2000 and HiSeq™ 1000) and the MiSeq™ system from Illumina, Inc. The HiSeq™ system is based on massively parallel sequencing of millions of fragments using attachment of randomly fragmented genomic DNA to a planar, optically transparent surface and solid phase amplification to create a high density sequencing flow cell with millions of clusters, each containing about 1,000 copies of template per sq. cm. These templates are sequenced using four-color DNA sequencing-by-synthesis technology. The MiSeq™ system uses TruSeq™, Illumina's reversible terminator-based sequencing-by-synthesis.

Another example of a DNA sequencing platform is the QIAGEN GeneReader platform—a next generation sequencing (NGS) platform utilizing proprietary modified nucleotides whose 3′ OH groups are reversely terminated by a small moiety to perform sequencing-by-synthesis (SBS) in a massively parallel manner. Briefly, the sequencing templates are first clonally amplified on a solid surface (such as beads) to generate hundreds of thousands of identical copies for each individual sequencing template, denaturized to generate single-stranded sequencing templates, hybridized with sequencing primer, and then immobilized on the flow cell. The immobilized sequencing templates are then subjected to a nucleotide incorporation reaction in a reaction mix that includes modified nucleotides with a cleavable 3′ blocking group that enables the incorporation and detection of only one specific nucleotide onto each sequencing template in each cycle. See U.S. Pat. Nos. 6,664,079; 8,612,161; and 8,623,598, each of which is incorporated by reference herein.

Another example of a DNA sequencing platform is the Ion Torrent PGM™ sequencer (Thermo Fisher) and the Ion Torrent Proton™ Sequencer (Thermo Fisher), which are ion-based sequencing systems that sequence nucleic acid templates by detecting ions produced as a byproduct of nucleotide incorporation. Typically, hydrogen ions are released as byproducts of nucleotide incorporations occurring during template-dependent nucleic acid synthesis by a polymerase. The Ion Torrent PGM™ sequencer and Ion Proton™ Sequencer detect the nucleotide incorporations by detecting the hydrogen ion byproducts of the nucleotide incorporations. The Ion Torrent PGM™ sequencer and Ion Torrent Proton™ sequencer include a plurality of nucleic acid templates to be sequenced, each template disposed within a respective sequencing reaction well in an array. The wells of the array are each coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor provides output signals indicative of nucleotide incorporation, which can be represented as voltage changes whose magnitude correlates with the H+ ion concentration in a respective well or reaction chamber. Different nucleotide types are flowed serially into the reaction chamber, and are incorporated by the polymerase into an extending primer (or polymerization site) in an order determined by the sequence of the template. Each nucleotide incorporation is accompanied by the release of H+ ions in the reaction well, along with a concomitant change in the localized pH. The release of H+ ions is registered by the FET of the sensor, which produces signals indicating the occurrence of the nucleotide incorporation. Nucleotides that are not incorporated during a particular nucleotide flow will not produce signals. The amplitude of the signals from the FET may also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers permit the instrument to resolve the sequence of many nucleic acid templates simultaneously. Further details regarding the compositions, design and operation of the Ion Torrent PGM™ sequencer can be found, for example, in U.S. Pat. Publn. Nos. 2009/0026082; 2010/0137143; and 2010/0282617, all of which are incorporated by reference herein in their entireties.

Another example of a DNA sequencing technique that can be used in the methods of the present disclosure is 454 sequencing (Roche) (Margulies et al., 2005). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Another example of a DNA sequencing technique that can be used in the methods of the present disclosure is SOLiD technology (Life Technologies, Inc.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

Another example of a DNA sequencing technique that can be used in the methods of the present disclosure is the IonTorrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection—no scanning, no cameras, no light—each nucleotide incorporation is recorded in seconds.

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

A further sequencing platform includes the CGA Platform (Complete Genomics). The CGA technology is based on preparation of circular DNA libraries and rolling circle amplification (RCA) to generate DNA nanoballs that are arrayed on a solid support (Drmanac et al. 2010). Complete genomics' CGA Platform uses a novel strategy called combinatorial probe anchor ligation (cPAL) for sequencing. The process begins by hybridization between an anchor molecule and one of the unique adapters. Four degenerate 9-mer oligonucleotides are labeled with specific fluorophores that correspond to a specific nucleotide (A, C, G, or T) in the first position of the probe. Sequence determination occurs in a reaction where the correct matching probe is hybridized to a template and ligated to the anchor using T4 DNA ligase. After imaging of the ligated products, the ligated anchor-probe molecules are denatured. The process of hybridization, ligation, imaging, and denaturing is repeated five times using new sets of fluorescently labeled 9-mer probes that contain known bases at the n+1, n+2, n+3, and n+4 positions.

A further sequencing platform includes nanopore sequencing (Oxford Nanopore). Nanopore detection arrays are described in US2011/0177498; US2011/0229877; US2012/0133354; WO2012/042226; WO2012/107778, and have been used for nucleic acid sequencing as described in US2012/0058468; US2012/0064599; US2012/0322679 and WO2012/164270, all of which are hereby incorporated by reference. A single molecule of DNA can be sequenced directly using a nanopore, without the need for an intervening PCR amplification step or a chemical labelling step or the need for optical instrumentation to identify the chemical label. Commercially available nanopore nucleic acid sequencing units are developed by Oxford Nanopore (Oxford, United Kingdom). The GridION™ system and miniaturised MinION™ device are designed to provide novel qualities in molecular sensing such as real-time data streaming, improved simplicity, efficiency and scalability of workflows and direct analysis of the molecule of interest. Using the Oxford Nanopore nanopore sequencing platform, an ionic current is passed through the nanopore by setting a voltage across this membrane. If an analyte passes through the pore or near its aperture, this event creates a characteristic disruption in current. Measurement of that current makes it possible to identify the molecule in question. For example, this system can be used to distinguish between the four standard DNA bases G, A, T and C, and also modified bases. It can be used to identify target proteins, small molecules, or to gain rich molecular information, for example to distinguish between the enantiomers of ibuprofen or study molecular binding dynamics. These nanopore arrays are useful for scientific applications specific for each analyte type; for example when sequencing DNA, the technology may be used for resequencing, de novo sequencing, and epigenetics.

### III. DEFINITIONS

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

As used herein, a nucleic acid “region” or “domain” is a consecutive stretch of nucleotides of any length.

“Incorporating,” as used herein, means becoming part of a nucleic acid polymer.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

The term “nucleic acid” or “polynucleotide” will generally refer to at least one molecule or strand of DNA, RNA, DNA-RNA chimera or a derivative or analog thereof, comprising at least one nucleobase, such as, for example, a naturally occurring purine or pyrimidine base found in DNA (e.g., adenine “A,” guanine “G,” thymine “T” and cytosine “C”) or RNA (e.g. A, G, uracil “U” and C). The term “nucleic acid” encompasses the terms “oligonucleotide” and “polynucleotide.” “Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adaptor” may also be used interchangeably with the terms “oligonucleotide” and “polynucleotide.” In addition, the term “adaptor” can indicate a linear adaptor (either single stranded or double stranded) or a stem-loop adaptor. These definitions generally refer to at least one single-stranded molecule, but in specific embodiments will also encompass at least one additional strand that is partially, substantially, or fully complementary to at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule. As used herein, a single stranded nucleic acid may be denoted by the prefix “ss,” a double-stranded nucleic acid by the prefix “ds,” and a triple stranded nucleic acid by the prefix “ts.”

A “nucleic acid molecule” or “nucleic acid target molecule” refers to any single-stranded or double-stranded nucleic acid molecule including standard canonical bases, hypermodified bases, non-natural bases, or any combination of the bases thereof. For example and without limitation, the nucleic acid molecule contains the four canonical DNA bases—adenine, cytosine, guanine, and thymine, and/or the four canonical RNA bases—adenine, cytosine, guanine, and uracil. Uracil can be substituted for thymine when the nucleoside contains a 2′-deoxyribose group. The nucleic acid molecule can be transformed from RNA into DNA and from DNA into RNA. For example, and without limitation, mRNA can be created into complementary DNA (cDNA) using reverse transcriptase and DNA can be created into RNA using RNA polymerase. A nucleic acid molecule can be of biological or synthetic origin. Examples of nucleic acid molecules include genomic DNA, cDNA, RNA, a DNA/RNA hybrid, amplified DNA, a pre-existing nucleic acid library, etc. A nucleic acid may be obtained from a human sample, such as blood, serum, plasma, cerebrospinal fluid, cheek scrapings, biopsy, semen, urine, feces, saliva, sweat, etc. A nucleic acid molecule may be subjected to various treatments, such as repair treatments and fragmenting treatments. Fragmenting treatments include mechanical, sonic, and hydrodynamic shearing. Repair treatments include nick repair via extension and/or ligation, polishing to create blunt ends, removal of damaged bases, such as deaminated, derivatized, abasic, or crosslinked nucleotides, etc. A nucleic acid molecule of interest may also be subjected to chemical modification (e.g., bisulfite conversion, methylation/demethylation), extension, amplification (e.g., PCR, isothermal, etc.), etc.

Nucleic acid(s) that are “complementary” or “complement(s)” are those that are capable of base-pairing according to the standard Watson-Crick, Hoogsteen or reverse Hoogsteen binding complementarity rules. As used herein, the term “complementary” or “complement(s)” may refer to nucleic acid(s) that are substantially complementary, as may be assessed by the same nucleotide comparison set forth above. The term “substantially complementary” may refer to a nucleic acid comprising at least one sequence of consecutive nucleobases, or semiconsecutive nucleobases if one or more nucleobase moieties are not present in the molecule, are capable of hybridizing to at least one nucleic acid strand or duplex even if less than all nucleobases do not base pair with a counterpart nucleobase. In certain embodiments, a “substantially complementary” nucleic acid contains at least one sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range therein, of the nucleobase sequence is capable of base-pairing with at least one single or double-stranded nucleic acid molecule during hybridization. In certain embodiments, the term “substantially complementary” refers to at least one nucleic acid that may hybridize to at least one nucleic acid strand or duplex in stringent conditions. In certain embodiments, a “partially complementary” nucleic acid comprises at least one sequence that may hybridize in low stringency conditions to at least one single or double-stranded nucleic acid, or contains at least one sequence in which less than about 70% of the nucleobase sequence is capable of base-pairing with at least one single or double-stranded nucleic acid molecule during hybridization.

The term “non-complementary” refers to nucleic acid sequence that lacks the ability to form at least one Watson-Crick base pair through specific hydrogen bonds.

The term “ligase” as used herein refers to an enzyme that is capable of joining the 3′ hydroxyl terminus of one nucleic acid molecule to a 5′ phosphate terminus of a second nucleic acid molecule to form a single molecule. The ligase may be a DNA ligase or RNA ligase. Examples of DNA ligases include E. coli DNA ligase, T4 DNA ligase, and mammalian DNA ligases.

“Sample” means a material obtained or isolated from a fresh or preserved biological sample or synthetically-created source that contains nucleic acids of interest. In certain embodiments, a sample is the biological material that contains the variable immune region(s) for which data or information are sought. Samples can include at least one cell, fetal cell, cell culture, tissue specimen, blood, serum, plasma, saliva, urine, tear, vaginal secretion, sweat, lymph fluid, cerebrospinal fluid, mucosa secretion, peritoneal fluid, ascites fluid, fecal matter, body exudates, umbilical cord blood, chorionic villi, amniotic fluid, embryonic tissue, multicellular embryo, lysate, extract, solution, or reaction mixture suspected of containing immune nucleic acids of interest. Samples can also include non-human sources, such as non-human primates, rodents and other mammals, other animals, plants, fungi, bacteria, and viruses.

As used herein in relation to a nucleotide sequence, “substantially known” refers to having sufficient sequence information in order to permit preparation of a nucleic acid molecule, including its amplification. This will typically be about 100%, although in some embodiments some portion of an adaptor sequence is random or degenerate. Thus, in specific embodiments, substantially known refers to about 50% to about 100%, about 60% to about 100%, about 70% to about 100%, about 80% to about 100%, about 90% to about 100%, about 95% to about 100%, about 97% to about 100%, about 98% to about 100%, or about 99% to about 100%.

### IV. KITS

The technology herein includes kits for creating libraries of target nucleic acids in a sample. A “kit” refers to a combination of physical elements. For example, a kit may include, for example, one or more components, such as Sinks and Probes, either with or without protector oligonucleotides, as well as specific primers, enzymes, reaction buffers, an instruction sheet, and other elements useful to practice the technology described herein. These physical elements can be arranged in any way suitable for carrying out the disclosure.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present disclosure also will typically include a means for containing the nucleic acids, and any other reagent containers in close confinement for commercial sale. Such containers may include injection or blow molded plastic containers into which the desired vials are retained.

A kit will also include instructions for employing the kit components as well the use of any other reagent not included in the kit. Instructions may include variations that can be implemented. It is contemplated that such reagents are embodiments of kits of the disclosure. Such kits, however, are not limited to the particular items identified above.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Allele Selective Enrichment Sequencing with 114-plex Non-pathogenic Panel

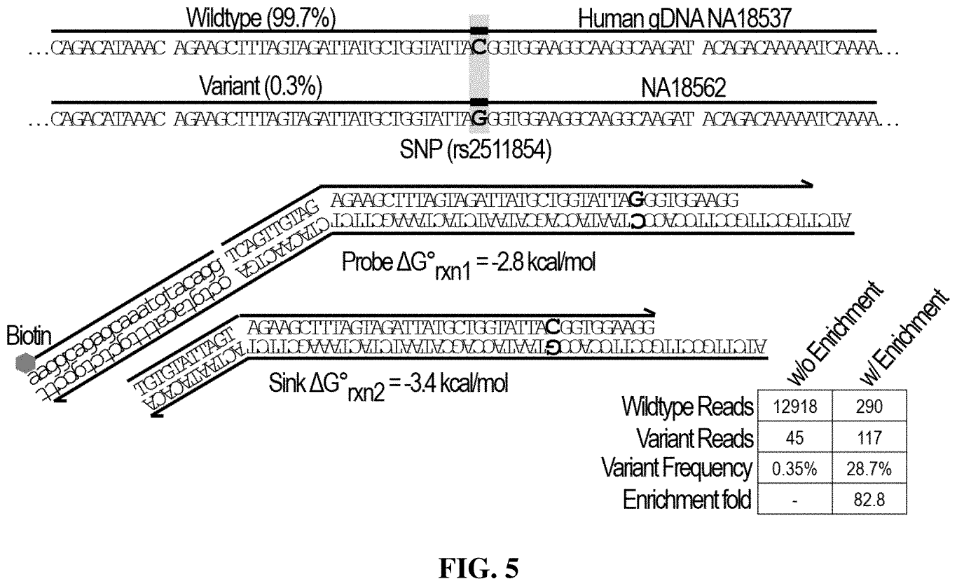

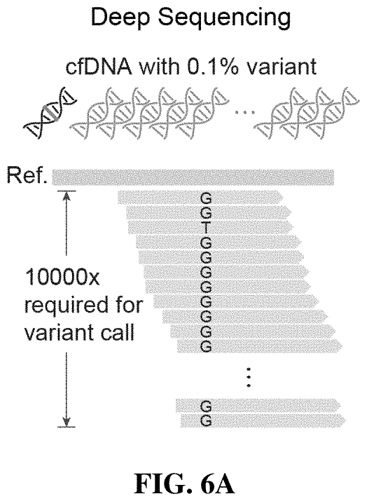

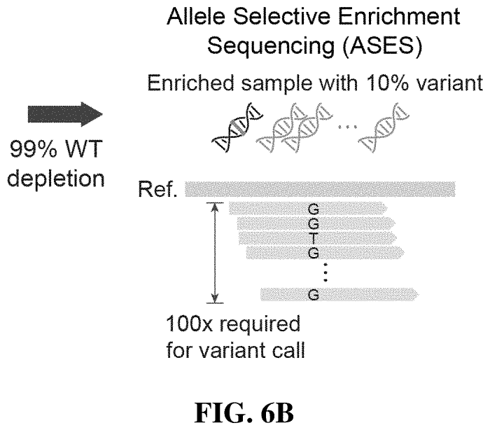

A 114-plex non-pathogenic panel has been completed using both positive and negative selection. See Tables 1-3 for the full sequence list used for the 114-plex panel. See FIG. 5 for an illustration of the sequences for the Variant, Wild-type, Probe, and Sink for one locus in the panel.

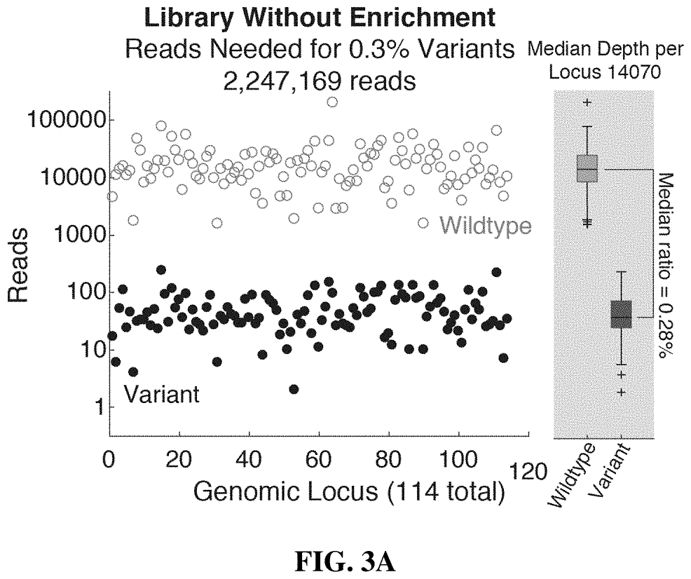

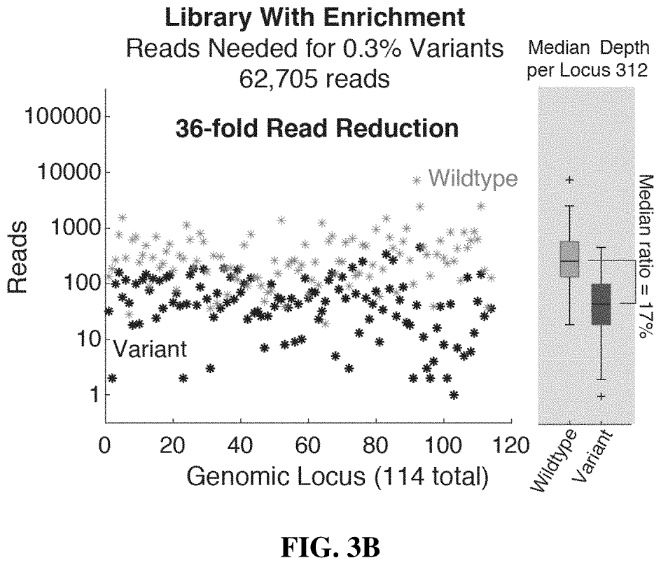

The genomic DNA input sample consisted of 498.5 ng NA18537 cell line DNA and 1.5 ng NA18562 cell line DNA. The sample had a 0.3% allele frequency in all single nucleotide polymorphisms (SNPs) in which both NA18537 and NA18562 are homozygous but differ from each other. In a 2.2M read library, with roughly 10,000× depth per locus, there are roughly 30 variant reads per locus, as expected for the 0.3% allele frequency sample (FIG. 3A). For enrichment, Probes were designed to NA18562 SNP alleles and Sinks were designed to NA18537 alleles. A 63 k read library produces similar reads for the variant for each locus, while the sequencing depth has been reduced 36-fold (FIG. 3B). Thus, sequencing cost can be reduced 36-fold while attaining similar information on rare mutations.

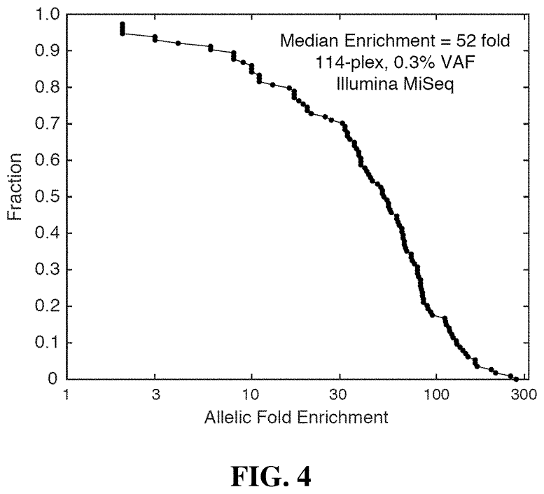

The distribution of fold-enrichment per locus for the 114-plex panel was analyzed. Median fold-enrichment observed was 52, and 90% of the Variants were enriched 8-fold or more (FIG. 4). Fold-enrichment can be improved through empirical optimization of Probe or Sink sequence, or of Probe protector and Sink protector stoichiometry.

In addition, panels using only negative selection, in which only wild-type alleles are depleted and thus the sequence of the variant need not be known, is also contemplated.

### Example 2—Allele Selective Enrichment Sequencing with 118-plex Non-pathogenic Panel

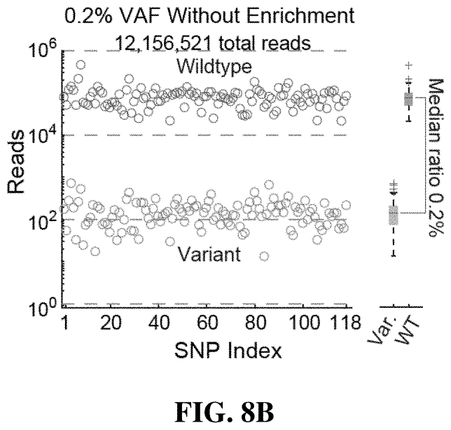

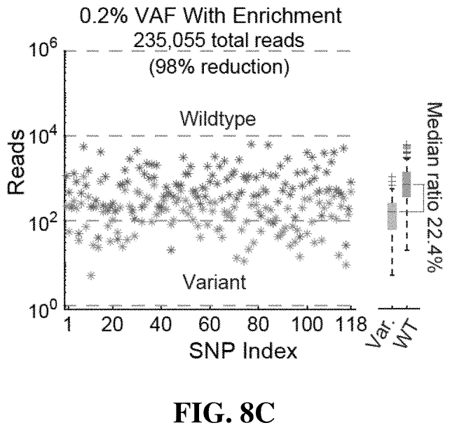

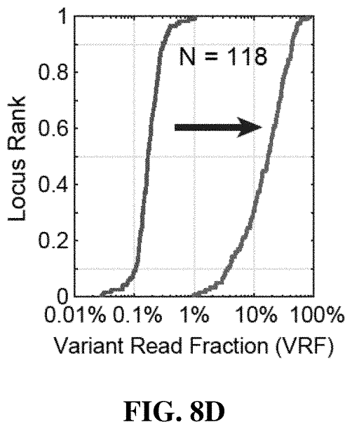

Amplicons were hybridized to variant-specific probes and wildtype-specific sinks to enrich variants of interest (FIG. 8A). Probes and sinks were implemented as partially double-stranded toehold probes, following thermodynamics and kinetics principles described in (Zhang, 2012). Probes were hybridized to universal biotinylated oligos to allow magnetic bead-based separation of amplicons bound to the probes. NGS reads were mapped to variant SNP alleles vs. wildtype alleles using standard deep sequencing. The input was 30 ng of a 0.2%:99.8% mixture of two human genomic DNA (gDNA) samples, NA18562 and NA18537. For each of the 118 SNPs in the panel, NA18562 and NA18537 are homozygous for different alleles; here the NA18537 alleles were considered the wildtype alleles, so the sample was 0.2% variant allele frequency (VAF) for all 118 SNPs. NGS results were consistent with expectations (FIG. 8B). NGS reads were mapped to the variant SNP alleles vs. wildtype alleles using ASES. The median ratio of variant reads to wildtype reads was 22.4%, indicating minor allele enrichment by a factor of roughly 100 over standard deep sequencing shown in FIG. 8B (FIG. 8C). FIG. 8D provides the distribution of variant read fraction (VRF, variant reads divided by total reads mapped to each SNP locus) for standard deep sequencing vs. ASES.

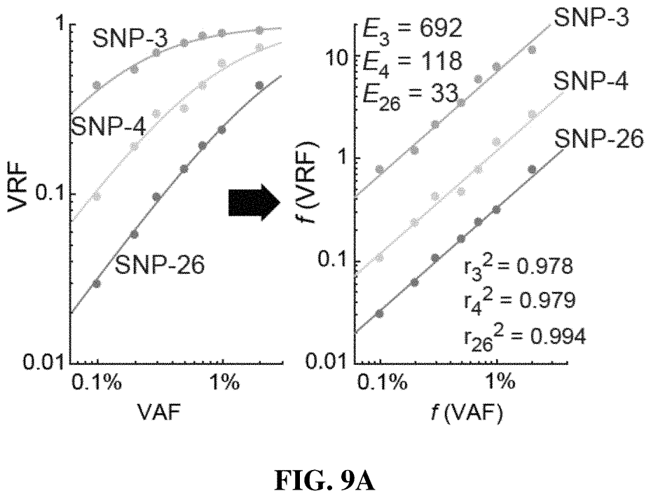

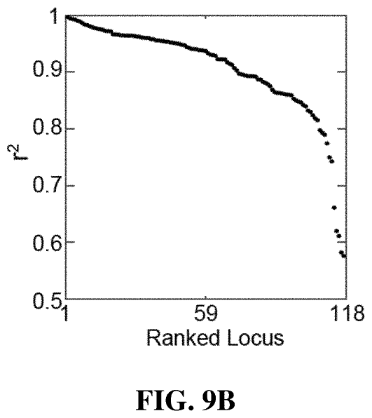

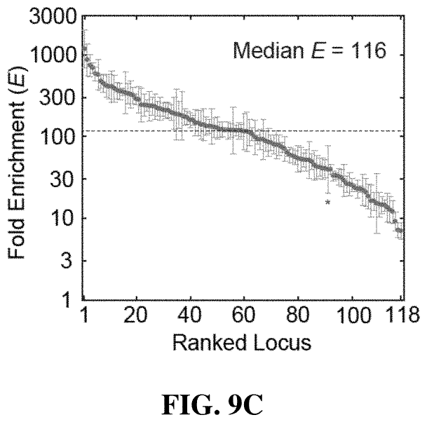

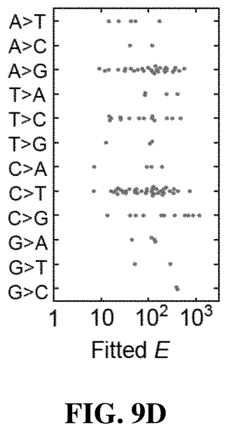

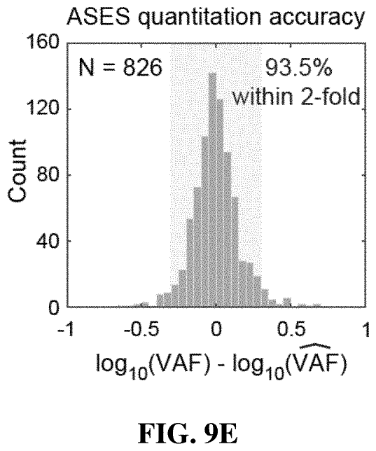

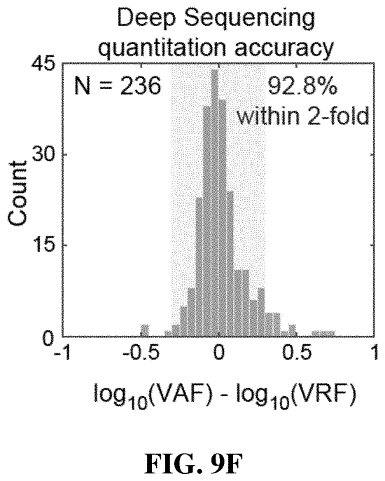

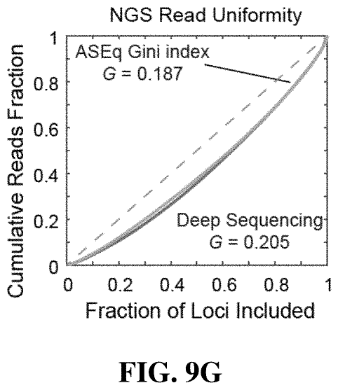

Estimation of sample VAF based on ASES VRF. FIG. 9A shows the consistent and predictable relationship between VRF, VAF, and fold-enrichment E. The dots represent experimental results from 7 different NGS libraries, where the input sample had known VAFs of 0.1%, 0.2%, 0.3%, 0.5%, 0.7%, 1%, 2%. The solid lines show theoretical curves for different values of E best-fitted to the three shown SNPs. The right panel plots VAF and VRF under non-linear transformations. The linear correlation constant r² of log₁₀(f(VRF)) vs. log₁₀(f(VAF)) indicates both confidence in the fitted parameter E and accuracy in inferring VAF from VRF. FIG. 9B shows the distribution of r² for the 118 SNP loci in the non-pathogenic ASES panel. One outlier SNP locus with r²<0 was omitted from the plot. FIG. 9C shows the distribution of best-t E. Error bars show the root mean square error (RMSE) of the linear t to the 7 data points for each SNP. Asterisk represents the one SNP in which the 0.1% VAF library did not have enough reads in the SNP locus to allow VRF quantitation; that SNP fitted E using the other 6 data points. FIG. 9D shows the distribution of fitted E for different base substitution types. One-way ANOVA indicates a p-value of 0.21, indicating that there is likely little to no sequence-based bias in E. FIG. 9E looks at the accuracy of inferred VAF based on observed VRF and fitted E. Here, leave-one-out was performed: E was tted to 6 out of 7 data points each SNP, and used to calculate the VAF of the last data point based on the VRF. VAF quantitation based on VRF for standard deep sequencing is shown in FIG. 9F. VAF quantitation accuracy was similar for deep sequencing and ASES. FIG. 9G shows the NGS read uniformity across the 118 different amplicons, visualized by the cumulative distribution plots of NGS reads vs. number of loci (Lorenz curve). The Gini index G denotes double the area between the cumulative distribution plot and the perfect equality dotted line (G=0 indicates perfect equality and G=1 indicates perfect inequality where all reads are in a single locus). The value of G for deep sequencing and ASES were observed to be similar.

### Example 3—Allele Selective Enrichment Sequencing with 112-plex Cancer Mutation Panel

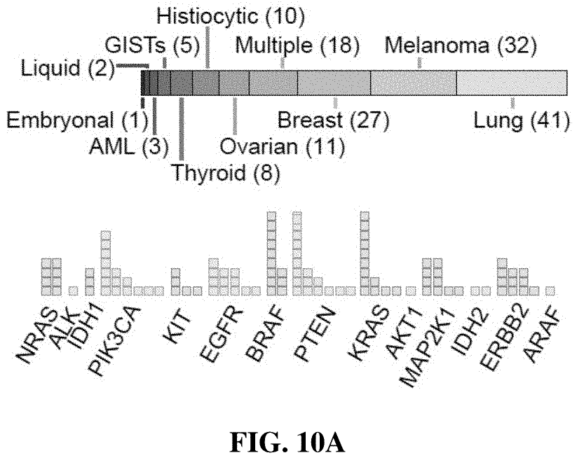

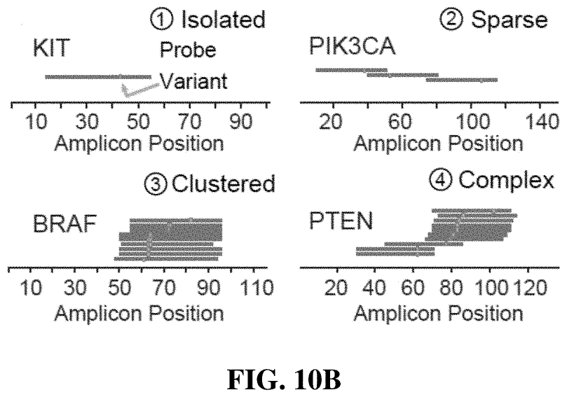

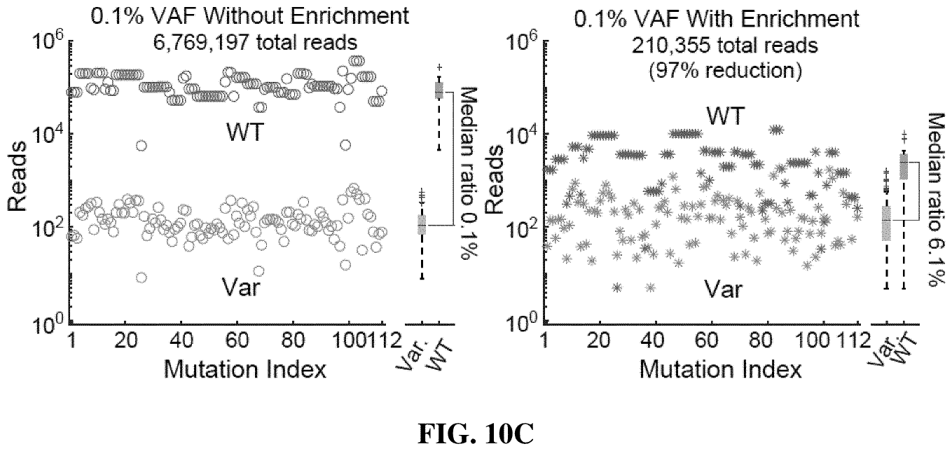

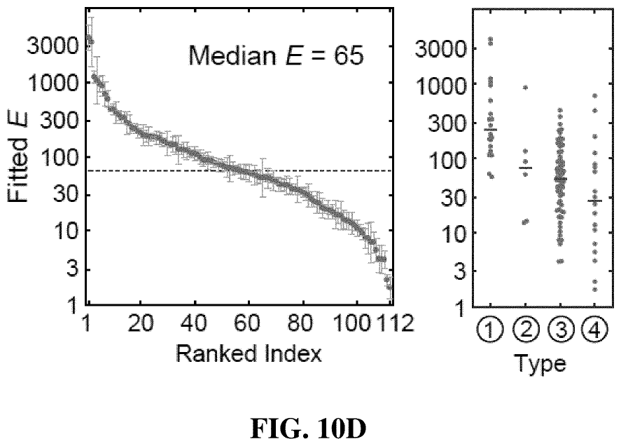

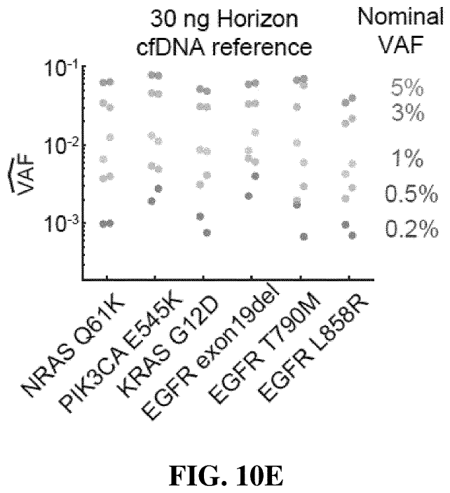

A cancer mutation panel, based on the National Comprehensive Cancer Network (NCCN) guidelines of actionable mutations, was generated. The panel covers 112 actionable mutations distributed across 42 amplicons (FIG. 10A). Shown are the distribution of mutations by corresponding cancer type, and the number of mutations profiled on each gene. FIG. 10B shows the distribution of potential cancer mutations on amplicons. Type 1, isolated mutations, are similar to the SNPs profiled in the NGS panel shown in FIGS. 9A-G. Type 2, sparse mutations, have multiple mutations colocalized on the same amplicon, but each mutation is sufficiently far from other mutations that we do not expect any interference between probes and sinks to each mutation. Type 3, clustered mutations, have multiple mutations all colocalized within a small window, so that the same wildtype sink can be applied to all of the mutations in the cluster. Type 4, complex groups, have clusters of mutations that are not sufficiently close to each other to be covered by the same wildtype sink, and require more complex and suboptimal design of probes and sinks. FIG. 10C shows NGS reads mapped to cancer mutations vs. wildtype using standard deep sequencing (left) and ASES (right). Here, the input was 30 ng of human genomic DNA (NA18537), spiked in with synthetic DNA molecules bearing each of the profiled mutations at 0.1% VAF. Note that because multiple hotspot mutations share the same wildtype, the same wildtype reads are repeatedly plotted at multiple different mutation loci; these wildtype reads are counted only once when considering total reads. FIG. 10D shows the distribution of observed fold-enrichment E, both in aggregate (left) and sorted by colocalization type (right). The mutation colocalization type appears to significantly affect E, with type 1 being the best performers and type 4 being the worst performers. FIG. 10E shows the validation of the cancer mutation panel on Horizon cfDNA reference samples (HD780 Multiplex I). Inferred VAF from ASES generally agreed with nominal VAFs provided by Horizon product insert.

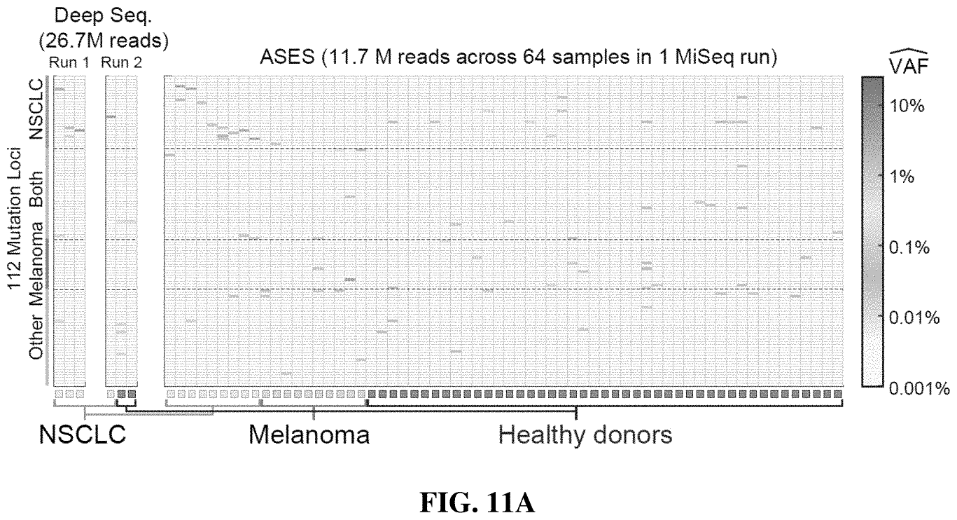

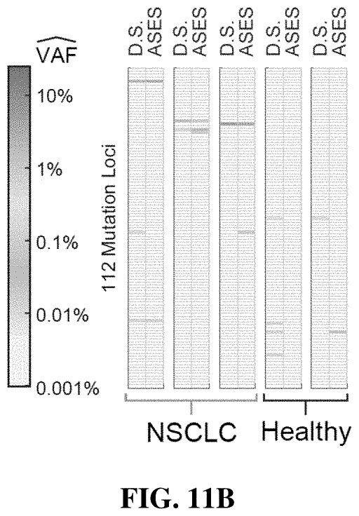

Validation of the ASES cancer mutation panel on clinical cfDNA samples is shown in FIGS. 11A-B. FIG. 11A provides a summary of called mutations in 6 samples by deep sequencing, and in 64 samples by ASES. Due to the reduced NGS reads required, all 64 samples for ASES were profiled using a single MiSeq run, while the 6 samples for deep sequencing required 2 separate MiSeq chips. Mutations were called for the ASES panel if the inferred VAF was more than 3 standard deviations greater than the median error rate, on a per-locus basis. Mutations were called for the deep sequencing panel if the inferred VAF was more than double the error rate, on a per-locus basis. A significant number of cancer mutations were found to be present even in cfDNA from healthy donors at between 0.01% and 1% inferred VAF. FIG. 11B provides a side-by-side comparison of ASES and deep sequencing on equal size aliquots of the same clinical cfDNA samples.

TABLE 1
Exemplary Probes

| SNP | Probe Complement | Probe Protector |
|---|---|---|
| rs2246745 | AAATAATCAGGAGAAGGAGATGGCATGTTTGTTGG | TGACTGAGAGAGCTCCTTGGAATCACCAACAAACA |
| | TGATTCCAAGGAGCTCTCTCAGTCATGATcctgtacatttg | TGCCATCTCCT (SEQ ID NO: 115) |
| | ctctgcctt (SEQ ID NO: 1) | |
| rs1805105 | GGAGCAGCGTCTCTGCCATCGTCCTCGTCCATGTCC | AAGTAGTTAATCTGGAAGTGTGACCAGGACATGGA |
| | TGGTCACACTTCCAGATTAACTACTTCGAAcctgtacattt | CGAGGACGATGGCAGAGA (SEQ ID NO: 116) |
| | gctctgcctt (SEQ ID NO: 2) | |
| rs3789806 | AGGTAAATATTTACCACCTCTTGGTGTTTATTTTAC | cGGTGCTCTTGTATATAGACGGTAAAATAAACACCA |
| | CGTCTATATACAAGAGCACCGCAAcctgtacatttgctctgcct | AGAGGT (SEQ ID NO: 117) |
| | t (SEQ ID NO: 3) | |
| rs9648696 | CTTTCAGTCAGATGTATATGCATTTGGGATTGTTCT | GCACATACTCATCAATTCATACAGAACAATCCCAA |
| | GTATGAATTGATGAGTATGTGCGGTTcctgtacatttgctctg | ATGCATATACA (SEQ ID NO: 118) |
| | cctt (SEQ ID NO: 4) | |
| rs116952709 | TATCTGCTAAGAAACAGACATCCATATACAGAGAT | TGATGTATCAAAAATCATCATTTTCATCTCTGTATA |
| | GAAAATGATGATTTTTGATACATCAACTGcctgtacatttg | TGGATGTCTGT (SEQ ID NO: 119) |
| | ctctgcctt (SEQ ID NO: 5) | |
| rs2511854 | ATCTTGCCTTGCCTTCCACCCTAATACCAGCATAAT | TCAGTTGTAGAGAAGCTTTAGTAGATTATGCTGGTA |
| | CTACTAAAGCTTCTCTACAACTGATACGcctgtacatttgct | TTAGGGTGGAAGG (SEQ ID NO: 120) |
| | ctgcctt (SEQ ID NO: 6) | |
| rs2510152 | TGTCCAGTGATATGGTTATAATGTGAAACAAAACT | TCTGCAGTAAAAGTGACCCAGGTGAGTTTTGTTTCA |
| | CACCTGGGTCACTTTTACTGCAGACCTTcctgtacatttgct | CATTATAACCA (SEQ ID NO: 121) |
| | ctgcctt (SEQ ID NO: 7) | |
| rs2066827 | ATTAAAGGCGCCGCCGGGCGGCTCCCGCTGCCATC | TCTCAGTGCGCAGGAGAGCCAGGATGGCAGCGGGA |
| | CTGGCTCTCCTGCGCACTGAGAGATAAcctgtacatttgctc | GCCGCCCGGCG (SEQ ID NO: 122) |
| | tgcctt (SEQ ID NO: 8) | |
| rs129974 | GATTTGCGTTCTGCACTATGACATAATTTGGCCTTC | ATATATATCGGAAAACAACTCACCCTGAAGGCCAA |
| | AGGGTGAGTTGTTTTCCGATATATATGTCTcctgtacattt | ATTATGTCATAGTGCA (SEQ ID NO: 123) |
| | gctctgcctt (SEQ ID NO: 9) | |
| rs2228422 | AGACTTCACCTTGTGATCTGCAGGGACTGACCTTAG | AGACTATTGCCAACACAACAACACTAAGGTCAGTC |
| | TGTTGTTGTGTTGGCAATAGTCTTGGAcctgtacatttgctct | CCTGCAGATCACA (SEQ ID NO: 124) |
| | gcctt (SEQ ID NO: 10) | |
| rs3738807 | AGAGATCTCCAAAGACACTCCACGGAATGAGGGCT | TGATATTAACATAAAGACAAGGGCAACAAGCCCTC |
| | TGTTGCCCTTGTCTTTATGTTAATATCAACAGcctgtac | ATTCCGTGGAGTGTCT (SEQ ID NO: 125) |
| | atttgctctgcctt (SEQ ID NO: 11) | |
| rs2294976 | CCTATACAATTGAGATGTTGGGGGAACCACAACAT | GCTCGTCACAGTCTGAATGAGTTATGTTGTGGTTCC |
| | AACTCATTCAGACTGTGACGAGCTTGAcctgtacatttgctc | CCCAACAT (SEQ ID NO: 126) |
| | tgcctt (SEQ ID NO: 12) | |
| rs2305351 | TTTTTATTGTATATGCATGCACATCCCAAGGACCAA | CACATGCGGTGTAGCTGGTCTCTTGGTCCTTGGGAT |
| | GAGACCAGCTACACCGCATGTGACGAcctgtacatttgctct | GTGCATG (SEQ ID NO: 127) |
| | gcctt (SEQ ID NO: 13) | |
| rs1630312 | GCAGGCATCAAAGTGCAGGACGTCCGGCTGAATGG | TGGCAGCTTTGGCCAGCTGCGGAGCCATTCAGCCG |
| | CTCCGCAGCTGGCCAAAGCTGCCATTCactgtacatttgct | GACGTCCTGCACTT (SEQ ID NO: 128) |
| | ctgcctt (SEQ ID NO: 14) | |
| rs10873531 | TCTGCATTCCCTGTCACTGCGTCACTGGCCTTCAGA | TAGCAATCAAGCACCTTGGCTCTGTCTGAAGGCCA |
| | CAGAGCCAAGGTGCTTGATTGCTACAATcctgtacatttgc | GTGACGCAGTGACAG (SEQ ID NO: 129) |
| | tctgcctt (SEQ ID NO: 15) | |
| rs8005905 | GCCCAAGTGTTTCTCTGGCATCTGTTGGTGTCTGGA | GAAGCACCAGTAGAGTGGTGGATCCAGACACCAAC |
| | TCCACCACTCTACTGGTGCTTCGTCAcctgtacatttgctctgc | AGATGCCAGAGAA (SEQ ID NO: 130) |
| | ctt (SEQ ID NO: 16) | |
| rs117396186 | GTTCAGATTTCACTGCCTCATGTTGATATTTCTTTCC | GTGAGAGATTAAACAGTCGTCATCTGGAAAGAAAT |
| | AGATGACGACTGTTTAATCTCTCACACCTcctgtacatttg | ATCAACATGAGGCA (SEQ ID NO: 131) |
| | ctctgcctt (SEQ ID NO: 17) | |
| rs34937835 | TAGACACATTGTCATCATGGACAGGCGGTGGATAC | ATAGTTAGGTATCAGCTAGACGGCACGTATCCACC |
| | GTGCCGTCTAGCTGATACCTAACTATAGAAcctgtacatt | GCCTGTCCATGATG (SEQ ID NO: 132) |
| | tgctctgcctt (SEQ ID NO: 18) | |
| rs17224367 | GCTTGTCTTTGAAACTTCTTGGCAAATCGGTTAAGA | AGGTATGAACCGTCGATTCCCAGATCTTAACCGATT |
| | TCTGGGAATCGACGGTTCATACCTCAATcctgtacatttgct | TGCCAAGAAGTTT (SEQ ID NO: 133) |
| | ctgcctt (SEQ ID NO: 19) | |
| rs2303428 | TACCTCCCATATTGGGGCCTACAGAACAAATTATAT | CGGTATCAATCTTGCTTTCTGATATAATTTGTTCTGT |
| | CAGAAAGCAAGATTGATACCGTTGAcctgtacatttgctctgc | AGGCCCCA (SEQ ID NO: 134) |
| | ctt (SEQ ID NO: 20) | |
| rs2229910 | TGCCAATGACCACAGTGTCGGGCCCCGCATCCAGT | GTTCTCCCACCACGCCCTCGTCACTGGATGCGGGGC |
| | GACGAGGGCGTGGTGGGAGAACCAGAcctgtacatttgctct | CCGACACTGTG (SEQ ID NO: 135) |
| | gcctt (SEQ ID NO: 21) | |
| rs200267496 | ATCCCCCTTAAAATCACGCTCACTTGCCGCGCATAG | tTCGTCGTCAACATCGAGATGGCCTATGCGCGGCAA |
| | GCCATCTCGATGTTGACGACGAACAGcctgtacatttgctctg | GTGAGCGTGAT (SEQ ID NO: 136) |
| | cctt (SEQ ID NO: 22) | |
| rs17334387 | GGGGAGGTGAAGCTGTCTATCTCCTACAAAAACAA | TTGACGTTAATATGATGAAGAGTTTATTGTTTTTGT |
| | TAAACTCTTCATCATATTAACGTCAATAAAcctgtacattt | AGGAGATAGACAGCTT (SEQ ID NO: 137) |
| | gctctgcctt (SEQ ID NO: 23) | |
| rs706713 | CGAGATTTTTTTCCTTCCAATATATTCTACATAAGT | TAGATCCATTCTGGGACTTTCCGGGAACTTATGTAG |
| | TCCCGGAAAGTCCCAGAATGGATCTAAAAGcctgtacat | AATATATTGGAAG (SEQ ID NO: 138) |
| | ttgctctgcctt (SEQ ID NO: 24) | |
| rs706714 | CTTTTCCTTAAAAAGAAAAAGAAAGGGAGTCATTA | ATCGGTGATACGTGGTTGCTTAATGACTCCCTTTCT |
| | AGCAACCACGTATCACCGATCATTGTcctgtacatttgctctg | TTTTC (SEQ ID NO: 139) |
| | cctt (SEQ ID NO: 25) | |
| rs290223 | TGTGTGTTATGATTTCTGTTGCAGAGTTGTGAAAAC | ACCTCTGGTGTTTTCACAGCCACGGTTTTCACAACT |
| | CGTGGCTGTGAAAACACCAGAGGTCTTCcctgtacatttgc | CTGCAACAGAA (SEQ ID NO: 140) |
| | tctgcctt (SEQ ID NO: 26) | |
| rs1230345 | GGCCCTGCAAATGCCCTCATCAGAAGCCCCGTTGC | TCTTCGAGTGCTCACTCCAGGAGGGCAACGGGGCT |
| | CCTCCTGGAGTGAGCACTCGAAGATCGAcctgtacatttgc | TCTGATGAGGGCATTT (SEQ ID NO: 141) |
| | tctgcctt (SEQ ID NO: 27) | |
| rs16754 | GCTACTCCAGGCACACGCCGCACATCCTGCAGGCA | CTGTATCGACTTCCTCTTACTCTCTGCCTGCAGGAT |
| | GAGAGTAAGAGGAAGTCGATACAGCCTAcctgtacatttg | GTGCGGCGTGTGC (SEQ ID NO: 142) |
| | ctctgcctt (SEQ ID NO: 28) | |
| rs6667687 | GATCTCTCCTGAGTCCTCACTAACAACAGGGGGTA | ACTTGGCTTGAAAACAATAAATCTACCCCCTGTTGT |
| | GATTTATTGTTTTCAAGCCAAGTATCAcctgtacatttgctct | TAGTGAGGAC (SEQ ID NO: 143) |
| | gcctt (SEQ ID NO: 29) | |
| rs3737639 | CCACTAGCCCTGGTTCAGGTCAGGGATGCCATGTC | AAGATTTGCCTGGGCCCCGACGACATGGCATCCCT |
| | GTCGGGGCCCAGGCAAATCTTTTCGcctgtacatttgctctgc | GACCTGAACCA (SEQ ID NO: 144) |
| | ctt (SEQ ID NO: 30) | |
| rs880724 | TGGCAGCCTCACTGTGCGGAGCATGGAGCCACACA | AATAACTTAACTGTGCCTACACCATGTGTGGCTCCA |
| | TGGTGTAGGCACAGTTAAGTTATTTATAcctgtacatttgct | TGCTCCGCACAGTG (SEQ ID NO: 145) |
| | ctgcctt (SEQ ID NO: 31) | |
| rs12475610 | AAGTACCCCAAAGTGTGAGGGCCTTCCCTCTGCCG | ACCGGACCGTTCTCGATGATGTGCGGCAGAGGGAA |
| | CACATCATCGAGAACGGTCCGGTCGCTcctgtacatttgctc | GGCCCTCACAC (SEQ ID NO: 146) |
| | tgcctt (SEQ ID NO: 32) | |
| rs867983 | GTGCTTCTGAAACTGTTATCTTCCCAGGAGCAATCT | CACACTAATATGCTTTCTATCCCCAGATTGCTCCTG |
| | GGGGATAGAAAGCATATTAGTGTGGAGAcctgtacatttg | GGAAGATAACAG (SEQ ID NO: 147) |
| | ctctgcctt (SEQ ID NO: 33) | |
| rs10207910 | GGGCCAGTCTTTAAATGCTTCCTGGAAAATGTTACT | ACTGCCAGAAGTTTATTTCATAGGTAGTAACATTTT |
| | ACCTATGAAATAAACTTCTGGCAGTggaccctgtacatttgct | CCAGGAAGCATTTAA (SEQ ID NO: 148) |
| | ctgcctt (SEQ ID NO: 34) | |
| rs1990856 | AAGGAAGCAGCGTGCAGTGCCATTCCTTCCTCCAC | TGACTGGATCCTAACGAGGCTTACGTGGAGGAAGG |
| | GTAAGCCTCGTTAGGATCCAGTCAACTTcctgtacatttgct | AATGGCACTGCAC (SEQ ID NO: 149) |
| | ctgcctt (SEQ ID NO: 35) | |
| rs73000450 | ATTGGGGGTATACTGGAAAAGTATTTTTGGTGTTGA | AAGGCTCGCTCACAGCCCAAACTTCAACACCAAAA |
| | AGTTTGGGCTGTGAGCGAGCCTTGTCAcctgtacatttgact | ATACTTTTCCAG (SEQ ID NO: 150) |
| | gcctt (SEQ ID NO: 36) | |
| rs75059082 | GGGATGTTTCTTGTCCTCGCTCAAGACAGAATTCGA | TCCACCGCCACTGGCTCACTCTCGAATTCTGTCTTG |
| | GAGTGAGCCAGTGGCGGTGGAaggacctgtacatttgactgcct | AGCGAGGAC (SEQ ID NO: 151) |
| | t (SEQ ID NO: 37) | |
| rs7648926 | TTTGACACCAATAAAACGGAGTGCCACTGAAGGGT | TCACTGGACTCTCCTCAGCTCAAAACCCTTCAGTGG |
| | TTTGAGCTGAGGAGAGTCCAGTGAACTAcctgtacatttgc | CACTCCGTT (SEQ ID NO: 152) |
| | tagcctt (SEQ ID NO: 38) | |
| rs2306253 | ACCGTACCTCTCCCCGACGTGGGCAGGCGTGAGTT | GATTATGTTGTTCTCTAAACTGACAACTCACGCCTG |
| | GTCAGTTTAGAGAACAACATAATCGTAGcctgtacatttgc | CCCACGTCGGGG (SEQ ID NO: 153) |
| | tagcctt (SEQ ID NO: 39) | |
| rs1316732 | CTGCTTCTAGGGTTGGGATCTCCCAGGGAAGACCG | GGCCTCGTTGCACATGGCAAGCCCGGTCTTCCCTGG |
| | GGCTTGCCATGTGCAACGAGGCCTTCTcctgtacatttgact | GAGATCCCAAC (SEQ ID NO: 154) |
| | gcctt (SEQ ID NO: 40) | |
| rs2672761 | GTCATTTTGCTGTTTGTTTTCTATATGCAGTATAAC | GCTGATTACATATTAAGAGACAAAAATGTTATACT |
| | ATTTTTGTCTCTTAATATGTAATCAGCTGATcctgtacatt | GCATATAGAAAACAAA (SEQ ID NO: 155) |
| | tgctagcctt (SEQ ID NO: 41) | |
| rs6882848 | TAGGAGACAGAGAATGTTCTGTGGGACCACAACCG | CCGGCATTAGAGCTCTTCTGTCTCGGTTGTGGTCCC |
| | AGACAGAAGAGCTCTAATGCCGGccGCcagtacatttgact | ACAGAACATT (SEQ ID NO: 156) |
| | gcctt (SEQ ID NO: 42) | |
| rs1465127 | CCTGTAACACACGCCCACAGGGGCTTCAGGAACTA | TCCAAGAGAAAAGGGTGAATGTTTATAGTTCCTGA |
| | TAAACATTCACCCTTTTCTCTTGGAtATGcctgtacatttgct | AGCCCCTGTGGGC (SEQ ID NO: 157) |
| | ctgcctt (SEQ ID NO: 43) | |
| rs1161899 | TATTTGATAAATTAACCCTAGAACAACTATCTGCAC | TGCGTTCCGTATGTGTTTCTGAGTGCAGATAGTTGT |
| | TCAGAAACACATACGGAACGCATTAGcctgtacatttgact | TCTAG (SEQ ID NO: 158) |
| | gcctt (SEQ ID NO: 44) | |
| rs4615440 | GAGCATCCTGAAGCAATTCTGTTTGTAATCCTGGAA | TGAGTATAGGTTCCCAGTTACTACTTCCAGGATTAC |
| | GTAGTAACTGGGAACCTATACTCACCAGcctgtacatttgc | AAACAGAATTGCT (SEQ ID NO: 159) |
| | tagcctt (SEQ ID NO: 45) | |
| rs9501710 | TGAATTATTTTTCTTCCCCTTCATTTTTGTTTAAGCT | TCGAATGGTACAAAAAACAATAGAGCTTAAACAAA |
| | CTATTGTTTTTTGTACCATTCGAgcCacctgtacatttgctagc | AATGAAGGG (SEQ ID NO: 160) |
| | ctt (SEQ ID NO: 46) | |
| rs6925983 | AGAACAATGTCCACATGTTTCCTCTGTGCCATTACT | CCGCCAATGGTAAGGGGACCATCTTAGTAATGGCA |
| | AAGATGGTCCCCTTACCATTGGCGGAAGCcctgtacattt | CAGAGGAAACATGT (SEQ ID NO: 161) |
| | gctctgcctt (SEQ ID NO: 47) | |
| rs2972171 | CATCCCACCCTGTCTCACTGGAGCCAGGATCCATG | TCTGAAGCCTAGCTCACGGGACCTCATGGATCCTG |
| | AGGTCCCGTGAGCTAGGCTTCAGAAGGCcctgtacatttgc | GCTCCAGTGAGACA (SEQ ID NO: 162) |
| | tctgcctt (SEQ ID NO: 48) | |
| rs62477557 | TCCATCCTAAAGGACTTACAGTTTCTTAGAATAACA | TCACTCCGAAAAATCACTCCATGTTATTCTAAGAAA |
| | TGGAGTGATTTTTCGGAGTGAcTGTcctgtacatttgctctgcct | CTGTAAGTC (SEQ ID NO: 163) |
| | t (SEQ ID NO: 49) | |
| rs4876049 | GAAAACAGTCAAAATGGCTGTCGACAATGAAATGG | ATTGCCTGAGTCTCAACTGATGTATCCATTTCATTG |
| | ATACATCAGTTGAGACTCAGGCAATGTGAcctgtacattt | TCGACAGCCA (SEQ ID NO: 164) |
| | gctctgcctt (SEQ ID NO: 50) | |
| rs1509186 | GAAAGACTAATAATTTTGCCCATGATCACCTCACC | AGAGCCGCCATTTCAGAGTGAGATGGTGAGGTGAT |
| | ATCTCACTCTGAAATGGCGGCTCTCAGGcctgtacatttgct | CATGGGC (SEQ ID NO: 165) |
| | ctgcctt (SEQ ID NO: 51) | |
| rs1876904 | AGTAGTCGGCATGGTGCTGAGCACCCTCCGGGAAC | TCGGTAGCCTTTCAGGTAGGGACGGTTCCCGGAGG |
| | CGTCCCTACCTGAAAGGCTACCGAgccgcctgtacatttgctct | GTGCTCAGCACCAT (SEQ ID NO: 166) |
| | gcctt (SEQ ID NO: 52) | |
| rs4880811 | AATTCTAGCTCCAAAATCTGGGCTCCTGACCACAAT | CACACTAATGCAAGGCACCTCTAACATTGTGGTCA |
| | GTTAGAGGTGCCTTGCATTAGTGTGGAGAcctgtacatttg | GGAGCCCAGATTT (SEQ ID NO: 167) |
| | ctctgcctt (SEQ ID NO: 53) | |
| rs75196694 | GAACCGTCACCAGGTCCTTTATTGCCTCTTCCAACA | ATCTTGCGATGGATAATTTCTATTGTTGGAAGAGGC |
| | ATAGAAATTATCCATCGCAAGATCGTGcctgtacatttgctc | AATAAAGGACCTG (SEQ ID NO: 168) |
| | tgcctt (SEQ ID NO: 54) | |
| rs2075545 | AGTCCTAACCTAGGTTACAGCCCATCACAGCTGGA | TGTATCAGGTTCTAAGCATCACCTGCTCCAGCTGTG |
| | GCAGGTGATGCTTAGAACCTGATACACCATcctgtacatt | ATGGGCTGTAAC (SEQ ID NO: 169) |
| | tgctctgcctt (SEQ ID NO: 55) | |
| rs60326265 | GTCAGGCTTAAGAGGCAGGGCCACCTAAACGTCTA | CGCCAATACCCTGTGTTCTCAGTAGACGTTTAGGTG |
| | CTGAGAACACAGGGTATTGGCGagcccctgtacatttgctctgc | GCCCTGCCT (SEQ ID NO: 170) |
| | ctt (SEQ ID NO: 56) | |
| rs953385 | TTCAGATATGACTAGGGAATGTTTAGAAAGTACAC | TCGCCTAGTCATGAAGCATGTGGCGTGTACTTTCTA |
| | GCCACATGCTTCATGACTAGGCGAggtccctgtacatttgctct | AACATTCC (SEQ ID NO: 171) |
| | gcctt (SEQ ID NO: 57) | |
| rs77983336 | CTCCAGGTATAGATGCAAGTAGGCTGGTAGATTTA | GAACTGAACGAGTTTGTCTCCTCATTAAATCTACCA |
| | ATGAGGAGACAAACTCGTTCAGTTCAGCCcctgtacattt | GCCTACTTGCA (SEQ ID NO: 172) |
| | gctctgcctt (SEQ ID NO: 58) | |
| rs1547149 | CTGGGTGTAAAGTTTCTGTGCAAACCTTTGCTACAG | CGGATGTCTTTGACTCGGCACGCACTGTAGCAAAG |
| | TGCGTGCCGAGTCAAAGACATCCGCGAGcctgtacatttgc | GTTTGCACAGAAA (SEQ ID NO: 173) |
| | tctgcctt (SEQ ID NO: 59) | |
| rs3117978 | GACCTGTAGTCACAAGTGTAGAGAGTTTGAGCTTC | TCGTGGTGTGCCTTTCTAAGTCGAAGCTCAAACTCT |
| | GACTTAGAAAGGCACACCACGATAcTcctgtacatttgactg | CTACACTTG (SEQ ID NO: 174) |
| | cat (SEQ ID NO: 60) | |
| rs9509962 | TTCACTGGCGATCAACAGTAACCAATAAAATTCAC | CCGGAAGGACTGTTGATTCATGAGTGAATTTTATTG |
| | TCATGAATCAACAGTCCTTCCGGTAGGcctgtacatttgctct | GTTACTGTTGAT (SEQ ID NO: 175) |
| | gcctt (SEQ ID NO: 61) | |
| rs7139530 | TCTCTGTAGTCAATTTGATTTTTATCAAGTTGCACT | AGTGATAGGGTCTTAAAATATTTAGTGCAACTTGAT |
| | AAATATTTTAAGACCCTATCACTAGAGcctgtacatttgact | AAAAATCAAA (SEQ ID NO: 176) |
| | gcctt (SEQ ID NO: 62) | |
| rs292476 | CAGCCTGTGTTCAGGATCTCACAAAGTCTCTCATGA | CATCGTATAGGTGCCCACAACTATTTTCATGAGAG |
| | AAATAGTTGTGGGCACCTATACGATGAATCcagtacatt | ACTTTGTGAGATCCTG (SEQ ID NO: 177) |
| | tgctctgcctt (SEQ ID NO: 63) | |
| rs3000029 | TTGTTCTCATCTCTCAGAAGCCCTTCTGTGGCCCAA | GTCTGGCGATGGTCAATAATGTTTGGGCCACAGAA |
| | ACATTATTGACCATCGCCAGACTTGCcagtacatttgactg | GGGCTTCTGA (SEQ ID NO: 178) |
| | cat (SEQ ID NO: 64) | |
| rs12434992 | GAAGCCTAGGTATGTAAATTACAGGCTTGCAGAAG | TCATGGCGAGCCACCTGCATTTACTTCTGCAAGCCT |
| | TAAATGCAGGTGGCTCGCCATGATGTCcagtacatttgctc | GTAATTTAC (SEQ ID NO: 179) |
| | tgcctt (SEQ ID NO: 65) | |
| rs1760904 | GGACGAGCCCCAGAAAAGTGGAAGAAGACTAATG | AACTATCATCGGGGCCAAGGTGGCATCATTAGTCT |
| | ATGCCACCTTGGCCCCGATGATAGTTCCAGcctgtacatt | TCTTCCACTTTTCTGG (SEQ ID NO: 180) |
| | tgctctgcctt (SEQ ID NO: 66) | |
| rs35567022 | TGCCCTCGTCCCTACTGGTAAGAGGCATAAGGTGG | TGGTGAGCACTTAGGCCCTTCCCCACCTTATGCCTC |
| | GGAAGGGCCTAAGTGCTCACCAGTCTcctgtacatttgctctg | TTACCAGTAGGG (SEQ ID NO: 181) |
| | cctt (SEQ ID NO: 67) | |
| rs12910624 | CTTAAAACTAAAACAGGAAAAAAAAATCAAAACC | CACGTAACATCGTGATTACTGATTTGTTACGGTTTT |
| | GTAACAAATCAGTAATCACGATGTTACGTGGCTGcct | GATTTTTTTTTC (SEQ ID NO: 182) |
| | gtacatttgctagcctt (SEQ ID NO: 68) | |
| rs34714665 | AAATCAGTAAAATGTTTACAAGCAATATCTTTTACG | GATGGTCGTCTTAGTTTTAAGATCGTAAAAGATATT |
| | ATCTTAAAACTAAGACGACCATCCTCAcctgtacatttgctc | GCTTGTAA (SEQ ID NO: 183) |
| | tgcctt (SEQ ID NO: 69) | |
| rs6576457 | CTACATAACAGAATTCAGTATGCAGTCATGATACA | TGGCTCGATAACTTTGCTGAGAGTATGTATCATGAC |
| | TACTCTCAGCAAAGTTATCGAGCCAttGGcctgtacatttgct | TGCATACTG (SEQ ID NO: 184) |
| | ctgcctt (SEQ ID NO: 70) | |
| rs2239669 | TCCTCTCAGTCTCTGAGCTCTGTAGAGGAGCCTCAG | CTTCTTCGGTTGCATCTGCCCCTGAGGCTCCTCTAC |
| | GGGCAGATGCAACCGAAGAAGGTATcctgtacatttgctctg | AGAGCTCAG (SEQ ID NO: 185) |
| | cctt (SEQ ID NO: 71) | |
| rs1698232 | ACACAAAACTAAAAGCACTTTTAATATTTCTTCAAA | TCTCTCGTTGGAATTGAAAGAAGTTTTGAAGAAAT |
| | ACTTCTTTCAATTCCAACGAGAGAGCGAcctgtacatttgct | ATTAAAAGTGC (SEQ ID NO: 186) |
| | ctgcctt (SEQ ID NO: 72) | |
| rs670962 | AGCCACTCCACTCCTAGGTATCTGCCCGAGAGACA | GACCGATGGTCCTTGTGCTTTCATGTCTCTCGGGCA |
| | TGAAAGCACAAGGACCATCGGTCGTCGcctgtacatttgct | GATACCTAGGAG (SEQ ID NO: 187) |
| | ctgcctt (SEQ ID NO: 73) | |
| rs58445115 | TGATCCCCAACAGAGAGAGGTACCCGGGATCTTCT | GATGCGCACATGAACCACGTCAGAAGATCCCGGGT |
| | GACGTGGTTCATGTGCGCATCGTTTcctgtacatttgctctgcc | ACCTCTCTCT (SEQ ID NO: 188) |
| | tt (SEQ ID NO: 74) | |
| rs59061318 | TCCGAATTCTCCAACTTTCCTCCCAGCACGGGTCTG | TGACTGAAGCCCGAGTCCCAAGGGCAGACCCGTGC |
| | CCCTTGGGACTCGGGCTTCAGTCATGTTcctgtacatttgct | TGGGAGGAAAGTT (SEQ ID NO: 189) |
| | ctgcctt (SEQ ID NO: 75) | |
| rs6506015 | TTTCCTTTCTTTCTTCCAAACTCCTCTTAATATTGGT | TGGCGTTCTAGGAGGTCAAAATACCAATATTAAGA |
| | ATTTTGACCTCCTAGAACGCCAGGGAcctgtacatttgctctg | GGAGTTTGGA (SEQ ID NO: 190) |
| | cctt (SEQ ID NO: 76) | |
| rs72634353 | ACATAGAAGGTGTTCAGTAAATATTTCCTGACAGT | TCCATACCTGCATTCATCAACTCCTACTGTCAGGAA |
| | AGGAGTTGATGAATGCAGGTATGGAgCAGcctgtacattt | ATATTTACTGA (SEQ ID NO: 191) |
| | gctctgcctt (SEQ ID NO: 77) | |
| rs55677929 | TCATGGCCGGTGGCCGGTTCTCACCCCTTTTGCTCC | TGTTAGGCTCTGCGTGTCTGTTAGGAGCAAAAGGG |
| | TAACAGACACGCAGAGCCTAACAcTGCcctgtacatttgctc | GTGAGAACCGGCCAC (SEQ ID NO: 192) |
| | tgcctt (SEQ ID NO: 78) | |
| rs6135141 | GGAGATACTGACAATTGCAAGTTGGGCTGATATGC | GGTTCTCGATATTTTCTGTTTTCATGCATATCAGCC |
| | ATGAAAACAGAAAATATCGAGAACCCGCAcctgtacattt | CAACTTGCAAT (SEQ ID NO: 193) |
| | gctctgcctt (SEQ ID NO: 79) | |
| rs2050980 | TAACAAAGACTAGCTTATACTACCCACACTTTCCTG | TGGAACTACCGAAAAGAAAAATGACAGGAAAGTG |
| | TCATTTTTCTTTTCGGTAGTTCCAATTGcctgtacatttgctct | TGGGTAGTATAA (SEQ ID NO: 194) |
| | gcctt (SEQ ID NO: 80) | |
| rs4815580 | AACATTTTGTTTTATAATCTGCGTCTGATAATACCG | CTCCATTGCAGAGTTTGTATATCGGTATTATCAGAC |
| | ATATACAAACTCTGCAATGGAGtTGGcctgtacatttgctctg | GCAGA (SEQ ID NO: 195) |
| | cctt (SEQ ID NO: 81) | |
| rs463397 | AGATGGTGAAGTAAAGATGAATAACATGAAGCAC | AGAATCAAGGCCAATAGCATTCAAATGTGCTTCAT |
| | ATTTGAATGCTATTGGCCTTGATTCTCGTTcctgtacattt | GTTATTCATCTT (SEQ ID NO: 196) |
| | gctctgcctt (SEQ ID NO: 82) | |
| rs7279689 | TAGTGATATTTCAATACATATAATGTATAGTGATCA | AATAATCGTACATTATGCTAATTACACTGATCACTA |
| | GTGTAATTAGCATAATGTACGATTATTAGACcctgtaca | TACATTATATG (SEQ ID NO: 197) |
| | tttgctctgcctt (SEQ ID NO: 83) | |
| rs5748211 | CTTTCTCTAGGTGCCGTACATGTTAGTGGGAGCTCC | ACCTACCTCTCCAATCCAGGAAATAAGGAGCTCCC |
| | TTATTTCCTGGATTGGAGAGGTAGGTCTtGcctgtacattt | ACTAACATGTACGG (SEQ ID NO: 198) |
| | gctctgcctt (SEQ ID NO: 84) | |
| rs79114187 | AACTCTCAGTTTGGGCCACTGCTCTCCAGTTGCCTG | TCACAGGTCGTAAGACTTAAAACTCCAGGCAACTG |
| | GAGTTTTAAGTCTTACGACCTGTGATAGGcctgtacatttg | GAGAGCAGTGGCC (SEQ ID NO: 199) |
| | ctctgcctt (SEQ ID NO: 85) | |
| rs13164 | ATGGCCAAGCCTTGGCTGTTGAGTAGGCACTGCCC | TGGCTCAACCATACAGCACAACTGGGCAGTGCCTA |
| | AGTTGTGCTGTATGGTTGAGCCATGGtcctgtacatttgctct | CTCAACAGCCAAG (SEQ ID NO: 200) |
| | gcctt (SEQ ID NO: 86) | |
| rs4633 | TCCCGGGCTCCGCATGCTGCAGCACATGGTTCAGG | AGTAAGGAATCAAGGAGCAGCGCATCCTGAACCAT |
| | ATGCGCTGCTCCTTGATTCCTTACTCCTAcctgtacatttgc | GTGCTGCAGCATGCGGA (SEQ ID NO: 201) |
| | tctgcctt (SEQ ID NO: 87) | |
| rs13303106 | GAAGGACCCCAGCTCCACCAACCAACAAAGGCACA | atAAGGTGGGTGGGACGGACTGTGCCTTTGTTGGTT |
| | GTCCGTCCCACCCACCTTATTGcctgtacatttgctctgcctt | GGTGGAGCT (SEQ ID NO: 202) |
| | (SEQ ID NO: 88) | |
| rs35273536 | GAAATAGACCCTCGACAGACCCAAAGGGGCCCACG | TACTTCTCTAACGTCACCACCGCATCACGTGGGCCC |
| | TGATGCGGTGGTGACGTTAGAGAAGTAAGGAcctgtac | CTTTGGGTCTGTC (SEQ ID NO: 203) |
| | atttgctctgcctt (SEQ ID NO: 89) | |
| rs77129670 | CCCAGATTTTGCTATTCCATACAGTTGACTGGACAT | GTACGCCACCAAAATGAGTTCATGTCCAGTCAACT |
| | GAACTCATTTTGGTGGCGTACGGAGGcctgtacatttgctctg | GTATGGAATA (SEQ ID NO: 204) |
| | cctt (SEQ ID NO: 90) | |
| rs17133064 | ATTCTGAAAGGAATGAAAATGGGGTTTAAATGTCC | GAAGATGCCCTCTGGACCTTAAGGACATTTAAACC |
| | TTAAGGTCCAGAGGGCATCTTCCAGTTcctgtacatttgctct | CCATTTTC (SEQ ID NO: 205) |
| | gcctt (SEQ ID NO: 91) | |
| rs1161901 | ATTCTGAAGATTTATCGTGAAAAAAAAAGAATGTA | TGTTGGATTCGATATTAATAAAAGATTGTACATTCT |
| | CAATCTTTTATTAATATCGAATCCAACAtactcctgtacattt | TTTTTTTTCAC (SEQ ID NO: 206) |
| | gctctgcctt (SEQ ID NO: 92) | |
| rs77474447 | CAACCTGCCCCTCCCTGACCCGGGGCCCCCTTTCCT | CAGGCGAGACTGGGCCCTGGAGGAAAGGGGGCCC |
| | CCAGGGCCCAGTCTCGCCTGCTGAAcctgtacatttgctctgc | CGGGTCAGGGAG (SEQ ID NO: 207) |
| | ctt (SEQ ID NO: 93) | |
| rs17756915 | GTTGACTTCTTTTAAAATATGATCTTCACAATTACC | CAGCTTCTCACAATTTGATTGGATGGTAATTGTGAA |
| | ATCCAATCAAATTGTGAGAAGCTGCGTTcctgtacatttgct | GATCATATT (SEQ ID NO: 208) |
| | ctgcctt (SEQ ID NO: 94) | |
| rs341697 | ATAGCTTTACCATTTTACCTTGCTCAATACGCACCC | gGAGATATCACGTGTCTCTTTGTCTGGGGTGCGTAT |
| | CAGACAAAGAGACACGTGATATCTCCCAAcctgtacattt | TGAGCAAGGTAA (SEQ ID NO: 209) |
| | gctctgcctt (SEQ ID NO: 95) | |
| rs10976019 | TTTGTTAGCAGGGTTGGATCTAACCAGTGATGTGCG | TGTAAGCTCACACTGACATGCCGCACATCACTGGTT |
| | GCATGTCAGTGTGAGCTTACATtTccctgtacatttgctctgcctt | AGATCCAAC (SEQ ID NO: 210) |
| | (SEQ ID NO: 96) | |
| rs76408959 | CCTCGTTACCTGCTTCTCATCTGTGATGCTCCCCAG | CTTATACCTCGGCAAATGTTGCAGAGATCTGGGGA |
| | ATCTCTGCAACATTTGCCGAGGTATAAGcggAcctgtac | GCATCACAGATGAGAAGC (SEQ ID NO: 211) |
| | atttgctctgcctt (SEQ ID NO: 97) | |
| rs9734804 | GCCTGGGGCCGGGCGGCAGGGGCGCGCAGGGTGG | CTACTAATAGAGGCCTCTGGGCCGCCACCCTGCGC |
| | CGGCCCAGAGGCCTCTATTAGTAGTAGACcctgtacattt | GCCCCTGCCGCCCGG (SEQ ID NO: 212) |
| | gctctgcctt (SEQ ID NO: 98) | |
| rs12792188 | GAGAGAGGGTGCTAGGCTGCTGGCCCAGCAAGGCC | GTCCCTGCTTCCCTTGAGGCCTTGCTGGGCCAGCAG |
| | TCAAGGGAAGCAGGGACATTAGTcctgtacatttgctctgcctt | CCTAG (SEQ ID NO: 213) |
| | (SEQ ID NO: 99) | |
| rs11611246 | GGGGTTGGGGGGGTGGTGTTGAGGTATGTGTAAGT | TAATTGGTGGCTATCATGAGCAATAGACTTACACA |
| | CTATTGCTCATGATAGCCACCAATTATCGAcctgtacattt | TACCTCAACACCACCCC (SEQ ID NO: 214) |
| | gctctgcctt (SEQ ID NO: 100) | |
| rs79782920 | AGGCGGGAACATAAACTAACAAAAAAGTATGTCAC | GTACTGCCAGAAACATGTCACTGTGCTGTGACATA |
| | AGCACAGTGACATGTTTCTGGCAGTACATCTcctgtaca | CTTTTTTGTTAGTTTATG (SEQ ID NO: 215) |
| | tttgctctgcctt (SEQ ID NO: 101) | |
| rs7989876 | TGATGGGAGCACACCCCCCAATGACCCTGCCCCCA | TATAAGGGCTTTTGCAGGTGTGGGGGCAGGGTCAT |
| | CACCTGCAAAAGCCCTTATAGACTcctgtacatttgctctgcctt | TGGGGGGTGT (SEQ ID NO: 216) |
| | (SEQ ID NO: 102) | |
| rs7982082 | TTAAAGCACATTAAAGCTCATTAGCCACTATGTCG | GGATGGACTTGATTAGATAAGGCCTCGACATAGTG |
| | AGGCCTTATCTAATCAAGTCCATCCTGACcctgtacatttg | GCTAATGAGC (SEQ ID NO: 217) |
| | ctctgcctt (SEQ ID NO: 103) | |
| rs77905703 | TAGTATATCATATAAAAATAAAGACATCACCCAAC | CACCTCGAAGGGTGATGTTTTTATGTTGGGTGATGT |
| | ATAAAAACATCACCCTTCGAGGTGTtggccctgtacatttgct | CTT (SEQ ID NO: 218) |
| | ctgcctt (SEQ ID NO: 104) | |
| rs59329234 | ATGTTGAACTCTTTTGTCAAAAGCCCCTTGTTGGAA | GTCATGCGAAGTCTCTAGACTTTTCCAACAAGGGG |
| | AAGTCTAGAGACTTCGCATGACATCTTcctgtacatttgctct | CTTTTGACA (SEQ ID NO: 219) |
| | gcctt (SEQ ID NO: 105) | |
| rs150926 | AAACCGTATGTGATCTAGCAATGGAGGAGAGGGTC | CTCTATGCAAGGTACTGGGGACTTCTGACCCTCTCC |
| | AGAAGTCCCCAGTACCTTGCATAGAGAaaatcctgtacattt | TCCATTGCTAGAT (SEQ ID NO: 220) |
| | gctctgcctt (SEQ ID NO: 106) | |
| rs12450330 | TCAAATTTCCCGTGATCATTACTGCCCATTTCCCAA | GCTTAAGATGTGCAATGAGATATTTTGGGAAATGG |
| | AATATCTCATTGCACATCTTAAGCCACGGcctgtacatttg | GCAGTAATGATCA (SEQ ID NO: 221) |
| | ctctgcctt (SEQ ID NO: 107) | |
| rs16948415 | GGTCATGATAAGTAAGCAGTGAAACAAAGTAGACA | AGGTAACGTTTTACTGATTCATATGTCTACTTTGTT |
| | TATGAATCAGTAAAACGTTACCTCTAGAcctgtacatttgct | TCACTGCT (SEQ ID NO: 222) |
| | ctgcctt (SEQ ID NO: 108) | |
| rs11878153 | CTTCACTCGCAGTAAATGTCTATTTCTCCTGTTTCAT | GTGTGTAGTTAACTCAACCTTTTTAATGAAACAGGA |
| | TAAAAAGGTTGAGTTAACTACACACAAGGcctgtacattt | GAAATAGACATTTAC (SEQ ID NO: 223) |
| | gctctgcctt (SEQ ID NO: 109) | |
| rs2279796 | CTCTGCCCACGGTATACCTGGGAGAGTGCAGGTCC | GACCGAACTCACCTTTCTGAAGGACCTGCACTCTCC |
| | TTCAGAAAGGTGAGTTCGGTCATCCTTcctgtacatttgctct | CAGGTATACC (SEQ ID NO: 224) |
| | gcctt (SEQ ID NO: 110) | |
| rs6074167 | TGATCATATGGTTTTTGTTTTTAATTCTGTTTATATG | TTATCGGACTGTAAGTGTGATTCACCATATAAACA |
| | GTGAATCACACTTACAGTCCGATAACACCGcctgtacatt | GAATTAAAAACAA (SEQ ID NO: 225) |
| | tgctctgcctt (SEQ ID NO: 111) | |
| rs2823170 | AGATAGATGACTTAGAGGCCCTTGGGTGTAACAGA | AGATGATTCTATTTGGGAAGACTGACTCTCTGTTAC |
| | GAGTCAGTCTTCCCAAATAGAATCATCTCATGAcctgt | ACCCAAGGGCCT (SEQ ID NO: 226) |
| | acatttgctctgcctt (SEQ ID NO: 112) | |
| rs9984697 | AATCTTCATAAAACCTCAGTGAATACTCTTTTTTCC | TTATAATTCGTTATTATTAAGATTTTTTTAACAGGA |
| | TGTTAAAAAAATCTTAATAATAACGAATTATAACC | AAAAAGAGTATTCACTGA (SEQ ID NO: 227) |
| | GGcctgtacatttgctctgcctt (SEQ ID NO: 113) | |
| rs17809319 | CTTGCTTATGAACACTAATTTCATATATAAAACAAA | GCAGTGGTTATCACAATAAATTTTTTGTTTTATATA |
| | AAATTTATTGTGATAACCACTGCATATGcctgtacatttgct | TGAAATTAGT (SEQ ID NO: 228) |
| | ctgcctt (SEQ ID NO: 114) | |

TABLE 2
Exemplary Sinks

| SNP | Sink Complement | Sink Protector |
|---|---|---|
| rs2246745 | AAATAATCAGGAGAAGGAGAAGGCATGTTTGTTGG | TGCTTCGAGCTCCTTGGAATCACCAACAAACATGC |
| | TGATTCCAAGGAGCTCGAAGCATAGG (SEQ ID NO: | CTTCTCCT (SEQ ID NO: 343) |
| | 229) | |
| rs1805105 | GGAGCAGCGTCTCTGCCATCGTCCTCATCCATGTCC | TACACTCTTGGAAGTGTGACCAGGACATGGATGAG |
| | TGGTCACACTTCCAAGAGTGTATTTAGT (SEQ ID | GACGATGGCAGAGA (SEQ ID NO: 344) |
| | NO: 230) | |
| rs3789806 | AGGTAAATATTTACCACGTCTTGGTGTTTATTTTAC | TATGATCTTGTATATAGACGGTAAAATAAACACCA |
| | CGTCTATATACAAGATCATACATTAC (SEQ ID NO: | AGACGT (SEQ ID NO: 345) |
| | 231) | |
| rs9648696 | CTTTCAGTCAGATGTATATGCATTTGGAATTGTTCT | TCCGTTTCATCAATTCATACAGAACAATTCCAAATG |
| | GTATGAATTGATGAAACGGAGAAT (SEQ ID NO: | CATATACA (SEQ ID NO: 346) |
| | 232) | |
| rs116952709 | TATCTGCTAAGAAACAGGCATCCATATACAGAGAT | GGTTATCAAAATCATCATTTTCATCTCTGTATATGG |
| | GAAAATGATGATTTTGATAACCTATA (SEQ ID NO: | ATGCCTGT (SEQ ID NO: 347) |
| | 233) | |
| rs2511854 | ATCTTGCCTTGCCTTCCACCGTAATACCAGCATAAT | TGTGTATTAGTAGAAGCTTTAGTAGATTATGCTGGT |
| | CTACTAAAGCTTCTACTAATACACACATC (SEQ ID | ATTACGGTGGAAGG (SEQ ID NO: 348) |
| | NO: 234) | |
| rs2510152 | TGTCCAGTGATATGGTTATCATGTGAAACAAAACT | TAGTCGAAAGTGACCCAGGTGAGTTTTGTTTCACAT |
| | CACCTGGGTCACTTTCGACTAGAAG (SEQ ID NO: | GATAACCA (SEQ ID NO: 349) |
| | 235) | |
| rs2066827 | ATTAAAGGCGCCGCCGGGCGGCTCCCGCTGACATC | CGAAGCGCAGGAGAGCCAGGATGTCAGCGGGAGC |
| | CTGGCTCTCCTGCGCTTCGCGCGAATG (SEQ ID NO: | CGCCCGGCG (SEQ ID NO: 350) |
| | 236) | |
| rs129974 | GATTTGCGTTCTGCACTATGACATCATTTGGCCTTC | TGCAATAAACAACTCACCCTGAAGGCCAAATGATG |
| | AGGGTGAGTTGTTTATTGCAGTAT (SEQ ID NO: 237) | TCATAGTGCA (SEQ ID NO: 351) |
| rs2228422 | AGACTTCACCTTGTGATCTGCAGGGACTGACCTTG | TCGGCCAACACAACAACACCAAGGTCAGTCCCTGC |
| | GTGTTGTTGTGTTGGCCGAAACC (SEQ ID NO: 238) | AGATCACA (SEQ ID NO: 352) |
| rs3738807 | AGAGATCTCCAAAGACACTCCACGGAATGAGGGCT | ACGCAAGACAAGGGCAACGAGCCCTCATTCCGTGG |
| | CGTTGCCCTTGTCTTGCGTGATT (SEQ ID NO: 239) | AGTGTCT (SEQ ID NO: 353) |
| rs2294976 | CCTATACAATTGAGATGGTGGGGGAACCACAACAT | TGTCCGCAGTCTGAATGAGTTATGTTGTGGTTCCCC |
| | AACTCATTCAGACTGCGGACAATTC (SEQ ID NO: | CACCAT (SEQ ID NO: 354) |
| | 240) | |
| rs2305351 | TTTTTATTGTATATGCATGCGCATCCCAAGGACCAA | AAGCGGTGTAGCTGGTCTCTTGGTCCTTGGGATGCG |
| | GAGACCAGCTACACCGCTTAGGT (SEQ ID NO: 241) | CATG (SEQ ID NO: 355) |
| rs1630312 | GCAGGCATCAAAGTGCACGACGTCCGGCTGAATGG | TACATTGGCCAGCTGCGGAGCCATTCAGCCGGACG |
| | CTCCGCAGCTGGCCAATGTACTTAGT (SEQ ID NO: | TCGTGCACTT (SEQ ID NO: 356) |
| | 242) | |
| rs10873531 | TCTGCATTCCCTGTCACCGCGTCACTGGCCTTCAGA | AGCAGGCACCTTGGCTCTGTCTGAAGGCCAGTGAC |
| | CAGAGCCAAGGTGCCTGCTTGTG (SEQ ID NO: 243) | GCGGTGACAG (SEQ ID NO: 357) |
| rs8005905 | GCCCAAGTGTTTCTCTGGCATCTGATGGTGTCTGGA | TCAAGCATAGTAGAGTGGTGGATCCAGACACCATC |
| | TCCACCACTCTACTATGCTTGAAATC (SEQ ID NO: | AGATGCCAGAGAA (SEQ ID NO: 358) |
| | 244) | |
| rs117396186 | GTTCAGATTTCACTGCCTCATGTTGATGTTTCTTTCC | CATGTATTAAGACAGTCGTCATCTGGAAAGAAACA |
| | AGATGACGACTGTCTTAATACATGGTTC (SEQ ID | TCAACATGAGGCA (SEQ ID NO: 359) |
| | NO: 245) | |
| rs34937835 | TAGACACATTGTCATCATGGACGGGCGGTGGATAC | GAAGACTCAGCTAGACGGCACGTATCCACCGCCCG |
| | GTGCCGTCTAGCTGAGTCTTCATAAT (SEQ ID NO: | TCCATGATG (SEQ ID NO: 360) |
| | 246) | |
| rs17224367 | GCTTGTCTTTGAAACTTCTTGGCAAGTCGGTTAAGA | CAATATCCGTCGATTCCCAGATCTTAACCGACTTGC |
| | TCTGGGAATCGACGGATATTGGAAAA (SEQ ID NO: | CAAGAAGTTT (SEQ ID NO: 361) |
| | 247) | |
| rs2303428 | TACCTCCCATATTGGGGCCTACAAAACAAATTATA | ATCATAATCTTGCTTTCTGATATAATTTGTTTTGTAG |
| | TCAGAAAGCAAGATTATGATCACAAA (SEQ ID NO: | GCCCCA (SEQ ID NO: 362) |
| | 248) | |
| rs2229910 | TGCCAATGACCACAGTGTCGGGCCCGGCATCCAGT | GCCCACCACGCCCTCGTCACTGGATGCCGGGCCCG |
| | GACGAGGGCGTGGTGGGCTCGATT (SEQ ID NO: | ACACTGTG (SEQ ID NO: 363) |
| | 249) | |
| rs200267496 | ATCCCCCTTAAAATCACACTCACTTGCCGCGCATAG | CAGCCAACATCGAGATGGCCTATGCGCGGCAAGTG |
| | GCCATCTCGATGTTGGCTGGACTA (SEQ ID NO: 250) | AGTGTGAT (SEQ ID NO: 364) |
| rs17334387 | GGGGAGGTGAAGCTGTCCATCTCCTACAAAAACAA | AGAATATGATGAAGAGTTTATTGTTTTTGTAGGAG |
| | TAAACTCTTCATCATATTCTCAACT (SEQ ID NO: | ATGGACAGCTT (SEQ ID NO: 365) |
| | 251) | |
| rs706713 | CGAGATTTTTTTCCTTCCAATATATTCTACGTAAGT | TCGATCATAGGGACTTTCCGGGAACTTACGTAGAA |
| | TCCCGGAAAGTCCCTATGATCGAAATC (SEQ ID NO: | TATATTGGAAG (SEQ ID NO: 366) |
| | 252) | |
| rs706714 | CTTTTCCTTAAAAAGAAAAAGAAAGGGAGTCATTA | CGTGTGATAAGTGGTTGCTTAATGACTCCCTTTCTT |
| | AGCAACCACTTATCACACGTATG (SEQ ID NO: 253) | TTTC (SEQ ID NO: 367) |
| rs290223 | TGTGTGTTATGATTTCTCTTGCAGAGTTGTGAAAAC | GAGCTCTTTTCACAGCCACGGTTTTCACAACTCTGC |
| | CGTGGCTGTGAAAAGAGCTCTGTA (SEQ ID NO: 254) | AAGAGAA (SEQ ID NO: 368) |
| rs1230345 | GGCCCTGCAAATGCCCTCAGCAGAAGCCCCGTTGC | CTTGCTTGCTCACTCCAGGAGGGCAACGGGGCTTCT |
| | CCTCCTGGAGTGAGCAAGCAAGAGTC (SEQ ID NO: | GCTGAGGGCATTT (SEQ ID NO: 369) |
| | 255) | |
| rs16754 | GCTACTCCAGGCACACGTCGCACATCCTGCAGGCA | GCGTGACTTCCTCTTACTCTCTGCCTGCAGGATGTG |
| | GAGAGTAAGAGGAAGTCACGCAAAC (SEQ ID NO: | CGACGTGTGC (SEQ ID NO: 370) |
| | 256) | |
| rs6667687 | GATCTCTCCTGAGTCCTCACTAACAACAGGGGGTT | GCTCTTGAAAACAATAAATCAACCCCCTGTTGTTAG |
| | GATTTATTGTTTTCAAGAGC (SEQ ID NO: 257) | TGAGGAC (SEQ ID NO: 371) |
| rs3737639 | CCACTAGCCCTGGTTCAGGTCAGGGATGCCATGTT | TATTTGCCTGGGCCCCGACAACATGGCATCCCTGAC |
| | GTCGGGGCCCAGGCAAATA (SEQ ID NO: 258) | CTGAACCA (SEQ ID NO: 372) |
| rs880724 | TGGCAGCCTCACTGTGCGGAGCATGGAGCCACACG | ACCACTGTGCCTACACCACGTGTGGCTCCATGCTCC |
| | TGGTGTAGGCACAGTGGT (SEQ ID NO: 259) | GCACAGTG (SEQ ID NO: 373) |
| rs12475610 | AAGTACCCCAAAGTGTGAGGGCCTTCCCTCTGCCA | AGCCATAAGTTCTCGATGATGTGTGGCAGAGGGAA |
| | CACATCATCGAGAACTTATGGCT (SEQ ID NO: 260) | GGCCCTCACAC (SEQ ID NO: 374) |
| rs867983 | GTGCTTCTGAAACTGTTATCTTCCCAGGAGCAATTT | ACTGATGCTTTCTATCCCCAAATTGCTCCTGGGAAG |
| | GGGGATAGAAAGCATCAGT (SEQ ID NO: 261) | ATAACAG (SEQ ID NO: 375) |
| rs10207910 | GGGCCAGTCTTTAAATGCTTCCTGGAAAATGTTGCT | ACAAGGTTTATTTCATAGGTAGCAACATTTTCCAGG |
| | ACCTATGAAATAAACCTTGT (SEQ ID NO: 262) | AAGCATTTAA (SEQ ID NO: 376) |
| rs1990856 | AAGGAAGCAGCGTGCAGTGCCATTCCTTCCTCCAG | ACCTAACCCTAACGAGGCTTACCTGGAGGAAGGAA |
| | GTAAGCCTCGTTAGGGTTAGGT (SEQ ID NO: 263) | TGGCACTGCAC (SEQ ID NO: 377) |
| rs73000450 | ATTGGGGGTATATTGGAAAAGTATTTTTGGTGTTGA | AGCTTGGCTCACAGCCCAAACTTCAACACCAAAAA |
| | AGTTTGGGCTGTGAGCCAAGCT (SEQ ID NO: 264) | TACTTTTCCA (SEQ ID NO: 378) |
| rs75059082 | GGGATGTTTCTTGTCCTCGTTCAAGACAGAATTCGA | GACTCCCCACTGGCTCACTCTCGAATTCTGTCTTGA |
| | GAGTGAGCCAGTGGGGAGTC (SEQ ID NO: 265) | ACGAGGAC (SEQ ID NO: 379) |
| rs7648926 | TTTGACACCAATAAAATGGAGTGCCACTGAAGGGT | TTCGTTCTCTCCTCAGCTCAAAACCCTTCAGTGGCA |
| | TTTGAGCTGAGGAGAGAACGAA (SEQ ID NO: 266) | CTCCATT (SEQ ID NO: 380) |
| rs2306253 | ACCGTACCTCTGCCCGACGTGGGCAGGCGTGAGTT | CTCTAATGTTCTCTAAACTGACAACTCACGCCTGCC |
| | GTCAGTTTAGAGAACATTAGAG (SEQ ID NO: 267) | CACGTCGGGC (SEQ ID NO: 381) |
| rs1316732 | CTGCTTCTAGGGTTGGGAACTCCCAGGGAAGACCG | TGTCGTGCACATGGCAAGCCCGGTCTTCCCTGGGA |
| | GGCTTGCCATGTGCACGACA (SEQ ID NO: 268) | GTTCCCAAC (SEQ ID NO: 382) |
| rs2672761 | GTCATTTTGCTGTTTGTTTTCTATATGCGGTATAAC | TCTCTCCTAAGAGACAAAAATGTTATACCGCATAT |
| | ATTTTTGTCTCTTAGGAGAGA (SEQ ID NO: 269) | AGAAAACAAA (SEQ ID NO: 383) |
| rs6882848 | TAGGAGACAGAGAATGTTCTGTGGGACCACAACCA | ACGGAGAGCTCTTCTGTCTTGGTTGTGGTCCCACAG |
| | AGACAGAAGAGCTCTCCGT (SEQ ID NO: 270) | AACATT (SEQ ID NO: 384) |
| rs1465127 | CCTGTAACACACGCCCACAGGGGCTTCAGGAACTG | CTGACTATAAAGGGTGAATGTTTACAGTTCCTGAA |
| | TAAACATTCACCCTTTATAGTCAG (SEQ ID NO: 271) | GCCCCTGTGGGC (SEQ ID NO: 385) |
| rs1161899 | TATTTGATAAATTAACCCTAGAACAACTATCTGCGC | GAGGATGGTATGTGTTTCTGAGCGCAGATAGTTGTT |
| | TCAGAAACACATACCATCCTC (SEQ ID NO: 272) | CTAG (SEQ ID NO: 386) |
| rs4615440 | GAGCATCCTGAAGCAATTCTGTTTGTAATCCTGGG | AATCTCATTCCCAGTTACTACTCCCAGGATTACAAA |
| | AGTAGTAACTGGGAATGAGATT (SEQ ID NO: 273) | CAGAATTGCT (SEQ ID NO: 387) |
| rs9501710 | TGAATTATTTTTCTTCCCTTTCATTTTTGTTTAAGCT | TCCATAACAAAAAACAATAGAGCTTAAACAAAAAT |
| | CTATTGTTTTTTGTTATGGA (SEQ ID NO: 274) | GAAAGG (SEQ ID NO: 388) |
| rs6925983 | AGAACAATGTCCACATGTTTCCTCTGTGCCATTATT | GTGATGGCAAGGGGACCATCTTAATAATGGCACAG |
| | AAGATGGTCCCCTTGCCATCAC (SEQ ID NO: 275) | AGGAAACATGT (SEQ ID NO: 389) |
| rs2972171 | CATCCCACCCTGTCTCACTGGAGCCAGGATCCATA | CAAGCTAGCTCACGGGACCTTATGGATCCTGGCTC |
| | AGGTCCCGTGAGCTAGCTTG (SEQ ID NO: 276) | CAGTGAGACA (SEQ ID NO: 390) |
| rs62477557 | TCCATCCTAAAGGACTTACGGTTTCTTAGAATAACA | TACTCTGAAAAATCACTCCATGTTATTCTAAGAAAC |
| | TGGAGTGATTTTTCAGAGTA (SEQ ID NO: 277) | CGTAAGTC (SEQ ID NO: 391) |
| rs4876049 | GAAAACAGTCAAAATGGCTGTCAACAATGAAATGG | ATGGTCTCTCAACTGATGTATCCATTTCATTGTTGA |
| | ATACATCAGTTGAGAGACCAT (SEQ ID NO: 278) | CAGCCA (SEQ ID NO: 392) |
| rs1509186 | GAAAGACTAATAATTTTGCCCATGATCACCTCACT | CTGTGCCATTTCAGAGTGAGATAGTGAGGTGATCA |
| | ATCTCACTCTGAAATGGCACAG (SEQ ID NO: 279) | TGGGC (SEQ ID NO: 393) |
| rs1876904 | AGTAGTCGGCATGGTGCTGAGCATCCTCCGGGAAC | TGCCTTTCAGGTAGGGACGGTTCCCGGAGGATGCT |
| | CGTCCCTACCTGAAAGGCA (SEQ ID NO: 280) | CAGCACCAT (SEQ ID NO: 394) |
| rs4880811 | AATTCTAGCTCCAAAATCTGGGCTCCTGACCACAG | CAATCGTAAAGGCACCTCTAACACTGTGGTCAGGA |
| | TGTTAGAGGTGCCTTTACGATTG (SEQ ID NO: 281) | GCCCAGATTT (SEQ ID NO: 395) |
| rs75196694 | GAACCGTCACCAGGTCCTTTATTGCCTCTTCCAATA | CACGGATGGATAATTTCTATTATTGGAAGAGGCAA |
| | ATAGAAATTATCCATCCGTG (SEQ ID NO: 282) | TAAAGGACCTG (SEQ ID NO: 396) |
| rs2075545 | AGTCCTAACCTAGGTTACAGCCCATCACAGCTGGG | ATTACATAACTAAGCATCACCTGCCCCAGCTGTGAT |
| | GCAGGTGATGCTTAGTTATGTAAT (SEQ ID NO: 283) | GGGCTGTAAC (SEQ ID NO: 397) |
| rs60326265 | GTCAGGCTTAAGAGGCAGGGCCACCTAAACGTCTG | AACCCTGTGTTCTCAGCAGACGTTTAGGTGGCCCTG |
| | CTGAGAACACAGGGTT (SEQ ID NO: 284) | CCT (SEQ ID NO: 398) |
| rs953385 | TTCAGATATGACTAGGGAATGTTTAGAAAGTACAG | TACTACTCATGAAGCATGTGGCCTGTACTTTCTAAA |
| | GCCACATGCTTCATGAGTAGTA (SEQ ID NO: 285) | CATTCC (SEQ ID NO: 399) |
| rs77983336 | CTCCAGGTATAGATGCAAGTAGGCTGGTAGATTTG | TCCGAAGTTTGTCTCCTCATCAAATCTACCAGCCTA |
| | ATGAGGAGACAAACTTCGGA (SEQ ID NO: 286) | CTTGCA (SEQ ID NO: 400) |
| rs1547149 | CTGGGTGTAAAGTTTCTGTGCAAACCTTTGCTACGG | ACGCGTGACTCGGCACGCACCGTAGCAAAGGTTTG |
| | TGCGTGCCGAGTCACGCGT (SEQ ID NO: 287) | CACAGAAA (SEQ ID NO: 401) |
| rs3117978 | GACCTGTAGTCACAAGTGTAGAGAGTTTGAGCTTT | TAGACTAATGTGCCTTTCTAAGTCAAAGCTCAAACT |
| | GACTTAGAAAGGCACATTAGTCTA (SEQ ID NO: 288) | CTCTACACTTG (SEQ ID NO: 402) |
| rs9509962 | TTCACTGGCGATCAACAGTAACTAATAAAATTCAC | CTGCATGTACTGTTGATTCATGAGTGAATTTTATTA |
| | TCATGAATCAACAGTACATGCAG (SEQ ID NO: 289) | GTTACTGTTGAT (SEQ ID NO: 403) |
| rs7139530 | TCTCTGTAGTCAATTTGATTTTTATCAAGTTGCATT | CTACCGTCTTAAAATATTTAATGCAACTTGATAAAA |
| | AAATATTTTAAGACGGTAG (SEQ ID NO: 290) | ATCAAA (SEQ ID NO: 404) |
| rs292476 | CAGCCTGTGTTCAGGATCTCACAGAGTCTCTCATGA | TGGCTTATGCCCACAACTATTTTCATGAGAGACTCT |
| | AAATAGTTGTGGGCATAAGCCA (SEQ ID NO: 291) | GTGAGATCCTG (SEQ ID NO: 405) |
| rs3000029 | TTGTTCTCATCTCTCAGATGCCCTTCTGTGGCCCAA | TTAGGATATGGTCAATAATGTTTGGGCCACAGAAG |
| | ACATTATTGACCATATCCTAA (SEQ ID NO: 292) | GGCATCTGA (SEQ ID NO: 406) |
| rs12434992 | GAAGCCTAGGTATGTAAATTATAGGCTTGCAGAAG | AATCGAAAGCCACCTGCATTTACTTCTGCAAGCCTA |
| | TAAATGCAGGTGGCTTTCGATT (SEQ ID NO: 293) | TAATTTAC (SEQ ID NO: 407) |
| rs1760904 | GGACGAGCCCCAGAAAAGTGGAAGAAGACTAATG | AAGAGTATGGGGGCCAAGGTGGCACCATTAGTCTT |
| | GTGCCACCTTGGCCCCCATACTCTT (SEQ ID NO: | CTTCCACTTTTCTGG (SEQ ID NO: 408) |
| | 294) | |
| rs35567022 | TGCCCTCGGCCCTACTGGTAAGAGGCATAAGGTGG | ATGAATAATTCACTTAGGCCCTTCCCCACCTTATGC |
| | GGAAGGGCCTAAGTGAATTATTCAT (SEQ ID NO: | CTCTTACCAGTAGGG (SEQ ID NO: 409) |
| | 295) | |
| rs12910624 | CTTAAAACTAAAACAGGAAAAAAAAATCAAAACC | TGGATCCAATTACTGATTTGTTATGGTTTTGATTTTT |
| | ATAACAAATCAGTAATTGGATCCA (SEQ ID NO: 296) | TTTTC (SEQ ID NO: 410) |
| rs34714665 | AAATCAGTAAAATGTTTACAAGCAATATCTTTTATG | AGCGCTCTTAGTTTTAAGATCATAAAAGATATTGCT |
| | ATCTTAAAACTAAGAGCGCT (SEQ ID NO: 297) | TGTAA (SEQ ID NO: 411) |
| rs6576457 | CTACATAACAGAATTCAGTATGCAGTCATGATACG | GAACTCAACTTTGCTGAGAGTACGTATCATGACTG |
| | TACTCTCAGCAAAGTTGAGTTC (SEQ ID NO: 298) | CATACTG (SEQ ID NO: 412) |
| rs2239669 | TCCTCTCAGTCTCTGAGCTCTGTAGAGGAGCCTCGG | TCTAAGGTTGCATCTGCCCCCGAGGCTCCTCTACAG |
| | GGGCAGATGCAACCTTAGA (SEQ ID NO: 299) | AGCTCAG (SEQ ID NO: 413) |
| rs1698232 | ACACAAAACTAAAAGCACTTTTAATATTTCTTCAG | TCTCCGAATTGAAAGAAGTTCTGAAGAAATATTAA |
| | AACTTCTTTCAATTCGGAGA (SEQ ID NO: 300) | AAGTGC (SEQ ID NO: 414) |
| rs670962 | AGCCACTCCACTCCTAGGTATCTGCCCAAGAGACA | CCAGTGTCCTTGTGCTTTCATGTCTCTTGGGCAGAT |
| | TGAAAGCACAAGGACACTGG (SEQ ID NO: 301) | ACCTAGGAG (SEQ ID NO: 415) |
| rs58445115 | TGATCCCCAACAGAGAGAGGTACCTGGGATCTTCT | TGACACACATGAACCACGTCAGAAGATCCCAGGTA |
| | GACGTGGTTCATGTGTGTCA (SEQ ID NO: 302) | CCTCTCTCT (SEQ ID NO: 416) |
| rs59061318 | TCCGAATTCTCCAACTTTCCTCCCAGCAAGGGTCTG | TCATCGCCCGAGTCCCAAGGGCAGACCCTTGCTGG |
| | CCCTTGGGACTCGGGCGATGA (SEQ ID NO: 303) | GAGGAAAGTT (SEQ ID NO: 417) |
| rs6506015 | TTTCCTTTCTTTCTTCCAAACTCCTGTTAATATTGGT | CCACAGTTAGGAGGTCAAAATACCAATATTAACAG |
| | ATTTTGACCTCCTAACTGTGG (SEQ ID NO: 304) | GAGTTTGGA (SEQ ID NO: 418) |
| rs72634353 | ACATAGAAGGTGTTCAGTAAATATTTCCTGACTGT | ACGCACATTCATCAACTCCTACAGTCAGGAAATAT |
| | AGGAGTTGATGAATGTGCGT (SEQ ID NO: 305) | TTACTGA (SEQ ID NO: 419) |
| rs55677929 | TCATGGCCGGTGGCCGGTTCTCACCCCTTTTGCTTC | TCGCTCTGCGTGTCTGTTAGAAGCAAAAGGGGTGA |
| | TAACAGACACGCAGAGCGA (SEQ ID NO: 306) | GAACCGGCCAC (SEQ ID NO: 420) |
| rs6135141 | GGAGATACTGACAATTGCAAGTTGGGCTGATATGT | TGCCGTATTTTCTGTTTTCATACATATCAGCCCAAC |
| | ATGAAAACAGAAAATACGGCA (SEQ ID NO: 307) | TTGCAAT (SEQ ID NO: 421) |
| rs2050980 | TAACAAAGACTAGCTTATACTACCCACGCTTTCCTG | TGAACCGAAAAGAAAAATGACAGGAAAGCGTGGG |
| | TCATTTTTCTTTTCGGTTCA (SEQ ID NO: 308) | TAGTATAA (SEQ ID NO: 422) |
| rs4815580 | AACATTTTGTTTTATAATCTGCGTCTGATAATACTG | CTAGAGCAGAGTTTGTATATCAGTATTATCAGACG |
| | ATATACAAACTCTGCTCTAG (SEQ ID NO: 309) | CAGA (SEQ ID NO: 423) |
| rs463397 | AGATGGTGAAGTAAAGATGAATAACATGAAGCAC | TCAGAAGCCAATAGCATTCAAACGTGCTTCATGTT |
| | GTTTGAATGCTATTGGCTTCTGA (SEQ ID NO: 310) | ATTCATCTT (SEQ ID NO: 424) |
| rs7279689 | TAGTGATATTTCAATACATATAATGTATAGTTATCA | TGTACTTTATGCTAATTACACTGATAACTATACATT |
| | GTGTAATTAGCATAAAGTACA (SEQ ID NO: 311) | ATATG (SEQ ID NO: 425) |
| rs5748211 | CTTTCTCTAGGTGCCGTACATGTTAGTGGGGGCTCC | GAGCTTCAATCCAGGAAATAAGGAGCCCCCACTAA |
| | TTATTTCCTGGATTGAAGCTC (SEQ ID NO: 312) | CATGTACGG (SEQ ID NO: 426) |
| rs79114187 | AACTCTCAGTTTGGGCCGCTGCTCTCCAGTTGCCTG | GTCGTCTAAGACTTAAAACTCCAGGCAACTGGAGA |
| | GAGTTTTAAGTCTTAGACGAC (SEQ ID NO: 313) | GCAGCGGCC (SEQ ID NO: 427) |
| rs13164 | ATGGCCAAGCCTTGGCTGTTGAGTAGGCAGTGCCC | TCCAACCATACAGCACAACTGGGCACTGCCTACTC |
| | AGTTGTGCTGTATGGTTGGA (SEQ ID NO: 314) | AACAGCCAAG (SEQ ID NO: 428) |
| rs4633 | TCCCGGGCTCCGCATGCTGCAGCACGTGGTTCAGG | TGAATCAAGGAGCAGCGCATCCTGAACCACGTGCT |
| | ATGCGCTGCTCCTTGATTCA (SEQ ID NO: 315) | GCAGCATGCGGA (SEQ ID NO: 429) |
| rs13303106 | GAAGGACCCCAGCTCCACCAACCAACAAAGGCAC | GGTGGGTGGGACGGACCGTGCCTTTGTTGGTTGGT |
| | GGTCCGTCCCACCCACC (SEQ ID NO: 316) | GGAGCT (SEQ ID NO: 430) |
| rs35273536 | GAAATAGACCCTCGACAGACCCAAAGGGGCCCAC | TCTAACGTCACCACCGCATCATGTGGGCCCCTTTGG |
| | ATGATGCGGTGGTGACGTTAGA (SEQ ID NO: 317) | GTCTGTC (SEQ ID NO: 431) |
| rs77129670 | CCCAGATTTTGCTAATCCATACAGTTGACTGGACAT | gTGTCTCACCAAAATGAGTTCATGTCCAGTCAACTG |
| | GAACTCATTTTGGTGAGACACATA (SEQ ID NO: 318) | TATGGATTA (SEQ ID NO: 432) |
| rs17133064 | ATTCTGAAAGGAATGAAAATGGGGTTTAAATGTCT | gtATAGTTACCCCTCTGGACCTTAAAGACATTTAAA |
| | TTAAGGTCCAGAGGGGTAACTATACTT (SEQ ID NO: | CCCCATTTTC (SEQ ID NO: 433) |
| | 319) | |
| rs1161901 | ATTCTGAAGATTTATCATGAAAAAAAAAGAATGTA | GACGGTTATTAATAAAAGATTGTACATTCTTTTTTT |
| | CAATCTTTTATTAATAACCGTC (SEQ ID NO: 320) | TTCAT (SEQ ID NO: 434) |
| rs77474447 | CAACCTGCCCCTCCCTGACCCGGGGCCCCCTTTTCT | TTCCATTCAAGACTGGGCCCTGGAGAAAAGGGGGC |
| | CCAGGGCCCAGTCTTGAATGGAA (SEQ ID NO: 321) | CCCGGGTCAGGGAG (SEQ ID NO: 435) |
| rs17756915 | GTTGACTTCTTTTAAAATATGATCTTCACAATTATC | caTCGTCTAACAATTTGATTGGATGATAATTGTGAA |
| | ATCCAATCAAATTGTTAGACGATGCT (SEQ ID NO: | GATCATATT (SEQ ID NO: 436) |
| | 322) | |
| rs341697 | ATAGCTTTACCATTTTACCTTGCTCAATACACACCC | GAGTGTGTCTCTTTGTCTGGGGTGTGTATTGAGCAA |
| | CAGACAAAGAGACACACTCA (SEQ ID NO: 323) | GGTAA (SEQ ID NO: 437) |
| rs10976019 | TTTGTTAGCAGGGTTGGATCTAACCAGTGATGTGTG | AAGGATTCACACTGACATGCCACACATCACTGGTT |
| | GCATGTCAGTGTGAATCCTT (SEQ ID NO: 324) | AGATCCAAC (SEQ ID NO: 438) |
| rs76408959 | CCTCGTTACCTGCTTCTCATCTGTGATGCTCCCCTG | TCATTATGAATGTTGCAGAGATCAGGGGAGCATCA |
| | ATCTCTGCAACATTCATAATGA (SEQ ID NO: 325) | CAGATGAGAAGC (SEQ ID NO: 439) |
| rs9734804 | GCCTGGGGCCGGGCGGCAGGGGCGCGCAGGGTGG | gTCTAAAGAGGCCTCTGGGCCCCCACCCTGCGCGCC |
| | GGGCCCAGAGGCCTCTTTAGACATG (SEQ ID NO: | CCTGCCGCCCGG (SEQ ID NO: 440) |
| | 326) | |
| rs12792188 | GAGAGAGGGTGCTAGGCTGCTGGCCCAGCAAGGC | CCCTGCTTCCCTTGACGCCTTGCTGGGCCAGCAGCC |
| | GTCAAGGGAAGCAGGGA (SEQ ID NO: 327) | TAG (SEQ ID NO: 441) |
| rs11611246 | GGGGTTGGGGGGGTGGTGTTGAGGTATGTGTAAGG | GGCGATATCATGAGCAATAGCCTTACACATACCTC |
| | CTATTGCTCATGATATCGCCG (SEQ ID NO: 328) | AACACCACCCC (SEQ ID NO: 442) |
| rs79782920 | AGGCGGGAACATAAACTAACAAAAAAGTATGTCAT | tTGCTCAACATGTCACTGTGCTATGACATACTTTTTT |
| | AGCACAGTGACATGTTGAGCAACCT (SEQ ID NO: | GTTAGTTTATG (SEQ ID NO: 443) |
| | 329) | |
| rs7989876 | TGATGGGAGCACACCCCCCAATGACCCTGCCCCCG | AGGGCTTTTGCAGGTGCGGGGGCAGGGTCATTGGG |
| | CACCTGCAAAAGCCCT (SEQ ID NO: 330) | GGGTGT (SEQ ID NO: 444) |
| rs7982082 | TTAAAGCACATTAAAGCTCATTAGCCACTATGTCA | TGAATCGATTAGATAAGGCCTTGACATAGTGGCTA |
| | AGGCCTTATCTAATCGATTCA (SEQ ID NO: 331) | ATGAGC (SEQ ID NO: 445) |
| rs77905703 | TAGTATATCATATAAAAATAAAGACATCACCCAAT | gTCCGAGGGTGATGTTTTTATATTGGGTGATGTCTT |
| | ATAAAAACATCACCCTCGGACTAA (SEQ ID NO: 332) | (SEQ ID NO: 446) |
| rs59329234 | ATGTTGAACTCTTTTGTCAAAAGCCCCTTGTTGGGA | AGACCATTAAGTCTCTAGACTTTCCCAACAAGGGG |
| | AAGTCTAGAGACTTAATGGTCT (SEQ ID NO: 333) | CTTTTGACA (SEQ ID NO: 447) |
| rs150926 | AAACCGTATGTGATCTAGCAATGGAGGAGAGGGTG | taTGCTCGTACTGGGGACTTCTCACCCTCTCCTCCAT |
| | AGAAGTCCCCAGTACGAGCATAAC (SEQ ID NO: | TGCTAGAT (SEQ ID NO: 448) |
| | 334) | |
| rs12450330 | TCAAATTTCCCGTGATCGTTACTGCCCATTTCCCAA | cCAACCATGCAATGAGATATTTTGGGAAATGGGCA |
| | AATATCTCATTGCATGGTTGGGTT (SEQ ID NO: 335) | GTAACGATCA (SEQ ID NO: 449) |
| rs16948415 | GGTCATGATAAGTAAGCAGTGAAACAAAGTAGAC | CGATTTTTTACTGATTCATACGTCTACTTTGTTTCAC |
| | GTATGAATCAGTAAAAAATCGA (SEQ ID NO: 336) | TGCT (SEQ ID NO: 450) |
| rs11878153 | CTTCACTCGCAGTAAATGTCTATTTCTCCTGTTTTAT | AGTGAGACTATCTCAACCTTTTTAATAAAACAGGA |
| | TAAAAAGGTTGAGATAGTCTCACT (SEQ ID NO: 337) | GAAATAGACATTTAC (SEQ ID NO: 451) |
| rs2279796 | CTCTGCCCACGGTATACCTGGGAGAGTGCAGGTCT | TAATAATAGACTCACCTTTCTGAAAGACCTGCACTC |
| | TTCAGAAAGGTGAGTCTATTATTAC (SEQ ID NO: | TCCCAGGTATACC (SEQ ID NO: 452) |
| | 338) | |
| rs6074167 | TGATCATATGGTTTTTGTTTTTAATTCTGTTTATGTG | taTTCGGTGAAGTGTGATTCACCACATAAACAGAAT |
| | GTGAATCACACTTCACCGAATAAG (SEQ ID NO: 339) | TAAAAACAA (SEQ ID NO: 453) |
| rs2823170 | AGATAGATGACTTAGAGGCCCTTGGGTGTAACAGT | GAAGAGTGGGAAGACTGACTCACTGTTACACCCAA |
| | GAGTCAGTCTTCCCACTCTTCAT (SEQ ID NO: 340) | GGGCCT (SEQ ID NO: 454) |
| rs9984697 | AATCTTCATAAAACCTCAGTGAATACTCTTTTTTAC | ACTATAATAGATTACTAGATTTTTTTAACAGTAAAA |
| | TGTTAAAAAAATCTAGTAATCTATTATAGT (SEQ ID | AAGAGTATTCACTGA (SEQ ID NO: 455) |
| | NO: 341) | |
| rs17809319 | CTTGCTTATGAACACTAATTTCATATATAAAACAGA | taTGATCCATCACAATAAATTTTCTGTTTTATATATG |
| | AAATTTATTGTGATGGATCATATA (SEQ ID NO: 342) | AAATTAGT (SEQ ID NO: 456) |

TABLE 3
Exemplary PCR Primers

| SNP | fPseq with adaptor | rPseq with adaptor |
|---|---|---|
| rs2246745 | tcgtcggcagcgtcagatgtgtataagagacagCCAAGCACATGGAT | gtctcgtgggctcggagatgtgtataagagacagGAGACAGGAAAGG |
| | CAGTGTT (SEQ ID NO: 457) | GAAGGAGT (SEQ ID NO: 571) |
| rs1805105 | tcgtcggcagcgtcagatgtgtataagagacagAGGGAAGGGCATAT | gtctcgtgggctcggagatgtgtataagagacagTGTCTCCAGGAGCA |
| | CTGGATAC (SEQ ID NO: 458) | GCTTC (SEQ ID NO: 572) |
| rs3789806 | tcgtcggcagcgtcagatgtgtataagagacagTTCACGCTTACCCAG | gtctcgtgggctcggagatgtgtataagagacagATCAACAACAGGGA |
| | GAGTT (SEQ ID NO: 459) | CCAGGTA (SEQ ID NO: 573) |
| rs9648696 | tcgtcggcagcgtcagatgtgtataagagacagAAGGTAACTGTCCA | gtctcgtgggctcggagatgtgtataagagacagTGTTCTAACAGGCA |
| | GTCATCAATTC (SEQ ID NO: 460) | CCAGAAGT (SEQ ID NO: 574) |
| rs116952709 | tcgtcggcagcgtcagatgtgtataagagacagGCTGTGTAGTTTCTA | gtctcgtgggctcggagatgtgtataagagacagACCACTCTGGCTGC |
| | AGGGTCG (SEQ ID NO: 461) | AAAGT (SEQ ID NO: 575) |
| rs2511854 | tcgtcggcagcgtcagatgtgtataagagacagCCCAGACGAGTACA | gtctcgtgggctcggagatgtgtataagagacagAAGTTATTGTTATTC |
| | GCTCA (SEQ ID NO: 462) | TTGATGGTTCTTTTGA (SEQ ID NO: 576) |
| rs2510152 | tcgtcggcagcgtcagatgtgtataagagacagAGAATCCTGATCTGA | gtctcgtgggctcggagatgtgtataagagacagGTTCCAATGAATTC |
| | CTGGCTT (SEQ ID NO: 463) | AATTATGCTGTCA (SEQ ID NO: 577) |
| rs2066827 | tcgtcggcagcgtcagatgtgtataagagacagCTTGCCCGAGTTCTA | gtctcgtgggctcggagatgtgtataagagacagCAAATGCGTGTCCT |
| | CTACAGA (SEQ ID NO: 464) | CAGAGTT (SEQ ID NO: 578) |
| rs129974 | tcgtcggcagcgtcagatgtgtataagagacagCTGGCTCTGTGCAGA | gtctcgtgggctcggagatgtgtataagagacagTCCTAGTTTCGTTGA |
| | ACTG (SEQ ID NO: 465) | TTGCAAGG (SEQ ID NO: 579) |
| rs2228422 | tcgtcggcagcgtcagatgtgtataagagacagGCCCAGATCGTGTGC | gtctcgtgggctcggagatgtgtataagagacagTCCACCATGGGAAA |
| | TC (SEQ ID NO: 466) | CCTGG (SEQ ID NO: 580) |
| rs3738807 | tcgtcggcagcgtcagatgtgtataagagacagGCTGGACTGGCTTCA | gtctcgtgggctcggagatgtgtataagagacagTTCACAGGGGCATG |
| | CAA (SEQ ID NO: 467) | TTTTAGC (SEQ ID NO: 581) |
| rs2294976 | tcgtcggcagcgtcagatgtgtataagagacagCTCCTCGTGGATCCA | gtctcgtgggctcggagatgtgtataagagacagAAAGGCAAAGAGG |
| | AAATTGC (SEQ ID NO: 468) | GCTTTGG (SEQ ID NO: 582) |
| rs2305351 | tcgtcggcagcgtcagatgtgtataagagacagGGTTTCAAGCCCTCT | gtctcgtgggctcggagatgtgtataagagacagCTGATCTATGATTCT |
| | GCA (SEQ ID NO: 469) | AAATTTTGCTGTCA (SEQ ID NO: 583) |
| rs1630312 | tcgtcggcagcgtcagatgtgtataagagacagCAGCCCAAGCCATTG | gtctcgtgggctcggagatgtgtataagagacagAACCTTGGAGATAA |
| | TCT (SEQ ID NO: 470) | CTCTGAAGGA (SEQ ID NO: 584) |
| rs10873531 | tcgtcggcagcgtcagatgtgtataagagacagAGCCTAAGCAATAT | gtctcgtgggctcggagatgtgtataagagacagGTCTCTGGAAACAG |
| | AAATGGCTGC (SEQ ID NO: 471) | CCCTTC (SEQ ID NO: 585) |
| rs8005905 | tcgtcggcagcgtcagatgtgtataagagacagCAGAGTAGAGTGGT | gtctcgtgggctcggagatgtgtataagagacagAGATTGTGTTTATG |
| | GGATCCA (SEQ ID NO: 472) | TTCCCAGCA (SEQ ID NO: 586) |
| rs117396186 | tcgtcggcagcgtcagatgtgtataagagacagTGAACACAGCCCAC | gtctcgtgggctcggagatgtgtataagagacagAACAACAACAACAG |
| | CTCA (SEQ ID NO: 473) | AAACCAGTTAG (SEQ ID NO: 587) |
| rs34937835 | tcgtcggcagcgtcagatgtgtataagagacagCTCTGCACTCCATGC | gtctcgtgggctcggagatgtgtataagagacagGCACCTTTCACAAT |
| | CAAC (SEQ ID NO: 474) | GGTTAAGG (SEQ ID NO: 588) |
| rs17224367 | tcgtcggcagcgtcagatgtgtataagagacagTGGAAGCTTTTGTAG | gtctcgtgggctcggagatgtgtataagagacagTGATAGAGTCGGTA |
| | AAGATGCA (SEQ ID NO: 475) | ACAATCTTGTAAG (SEQ ID NO: 589) |
| rs2303428 | tcgtcggcagcgtcagatgtgtataagagacagCAGTGTACAGTTTAG | gtctcgtgggctcggagatgtgtataagagacagCCCAATTTGGGCCA |
| | GACTAACAATCC (SEQ ID NO: 476) | TGAGT (SEQ ID NO: 590) |
| rs2229910 | tcgtcggcagcgtcagatgtgtataagagacagGTCATCAGTGGTGAG | gtctcgtgggctcggagatgtgtataagagacagCAGTTGTGTCCCTG |
| | GAGGA (SEQ ID NO: 477) | ACGG (SEQ ID NO: 591) |
| rs200267496 | tcgtcggcagcgtcagatgtgtataagagacagCCAAGCTACATCAGT | gtctcgtgggctcggagatgtgtataagagacagGTTTCCTTTTACTCC |
| | GATGTGG (SEQ ID NO: 478) | CTAGAGGTT (SEQ ID NO: 592) |
| rs17334387 | tcgtcggcagcgtcagatgtgtataagagacagTGTGCAGGCACTTAC | gtctcgtgggctcggagatgtgtataagagacagTCATGGTTTCATTTG |
| | CAAG (SEQ ID NO: 479) | TCCCTACA (SEQ ID NO: 593) |
| rs706713 | tcgtcggcagcgtcagatgtgtataagagacagGAAGCCAGGCCTGA | gtctcgtgggctcggagatgtgtataagagacagAGGAAGAGGCCGA |
| | AGAAA (SEQ ID NO: 480) | GGTG (SEQ ID NO: 594) |
| rs706714 | tcgtcggcagcgtcagatgtgtataagagacagACTGAAGCAGATGTT | gtctcgtgggctcggagatgtgtataagagacagCCCAGAACATAACG |
| | GAACAACA (SEQ ID NO: 481) | ACTCAACC (SEQ ID NO: 595) |
| rs290223 | tcgtcggcagcgtcagatgtgtataagagacagTCATTGGCCTCGTTT | gtctcgtgggctcggagatgtgtataagagacagCACAGGGGGATTAT |
| | TTCAGT (SEQ ID NO: 482) | GCTTCAC (SEQ ID NO: 596) |
| rs1230345 | tcgtcggcagcgtcagatgtgtataagagacagCCTGGTTGCTTGGCA | gtctcgtgggctcggagatgtgtataagagacagTGAAGGAAGGCCTG |
| | CA (SEQ ID NO: 483) | GAGAA (SEQ ID NO: 597) |
| rs16754 | tcgtcggcagcgtcagatgtgtataagagacagCTCCCTCAAGACCTA | gtctcgtgggctcggagatgtgtataagagacagCGTTTCTCACTGGTC |
| | CGTGA (SEQ ID NO: 484) | TCAGATG (SEQ ID NO: 598) |
| rs6667687 | tcgtcggcagcgtcagatgtgtataagagacaggttaaagacggcacttccaacag | gtctcgtgggctcggagatgtgtataagagacagtgacccttgccctggtaga |
| | (SEQ ID NO: 485) | (SEQ ID NO: 599) |
| rs3737639 | tcgtcggcagcgtcagatgtgtataagagacagtcctccgtggctctccc (SEQ | gtctcgtgggctcggagatgtgtataagagacagctgccctggagccactag |
| | ID NO: 486) | (SEQ ID NO: 600) |
| rs880724 | tcgtcggcagcgtcagatgtgtataagagacagagcttggggacacctctga | gtctcgtgggctcggagatgtgtataagagacagaccacgaacagcagaagca |
| | (SEQ ID NO: 487) | (SEQ ID NO: 601) |
| rs12475610 | tcgtcggcagcgtcagatgtgtataagagacaggatggttccagctgcgct (SEQ | gtctcgtgggctcggagatgtgtataagagacagtgtgtatcatcatctctaatttaaag |
| | ID NO: 488) | aaaaagtac (SEQ ID NO: 602) |
| rs867983 | tcgtcggcagcgtcagatgtgtataagagacagtccgatataagttaacaatgcaatg | gtctcgtgggctcggagatgtgtataagagacagtggccagccaagggga (SEQ |
| | tca (SEQ ID NO: 489) | ID NO: 603) |
| rs10207910 | tcgtcggcagcgtcagatgtgtataagagacagtgatcttatttatatattttcagtcattt | gtctcgtgggctcggagatgtgtataagagacagccgtgtgctccatcttacaatac |
| | gtcctac (SEQ ID NO: 490) | (SEQ ID NO: 604) |
| rs1990856 | tcgtcggcagcgtcagatgtgtataagagacaggctccaacatttcatccaggatttg | gtctcgtgggctcggagatgtgtataagagacagggcccagcgtgtgtatga |
| | (SEQ ID NO: 491) | (SEQ ID NO: 605) |
| rs73000450 | tcgtcggcagcgtcagatgtgtataagagacaggccattacacctaagcaccatcta | gtctcgtgggctcggagatgtgtataagagacagtctccatttgtagctgaattcttgtc |
| | c (SEQ ID NO: 492) | (SEQ ID NO: 606) |
| rs75059082 | tcgtcggcagcgtcagatgtgtataagagacagaaagctaaagcagagaatgaagt | gtctcgtgggctcggagatgtgtataagagacagtgtttttgtttttttaccactggctc |
| | tga (SEQ ID NO: 493) | (SEQ ID NO: 607) |
| rs7648926 | tcgtcggcagcgtcagatgtgtataagagacagaatatcatgtcctatttctcctcagct | gtctcgtgggctcggagatgtgtataagagacaggccaaacagtgttttgtagaccat |
| | (SEQ ID NO: 494) | t (SEQ ID NO: 608) |
| rs2306253 | tcgtcggcagcgtcagatgtgtataagagacagggagctgtgacaatgaaaatgca | gtctcgtgggctcggagatgtgtataagagacaggatcagggggcagaaggatg |
| | g (SEQ ID NO: 495) | (SEQ ID NO: 609) |
| rs1316732 | tcgtcggcagcgtcagatgtgtataagagacagccgtcaccgtggagtttcc | gtctcgtgggctcggagatgtgtataagagacagccctgctctgacaccagg |
| | (SEQ ID NO: 496) | (SEQ ID NO: 610) |
| rs2672761 | tcgtcggcagcgtcagatgtgtataagagacagtggaagagcttacatttaagtgatt | gtctcgtgggctcggagatgtgtataagagacagtgatactaccaaaataatcaaaa |
| | actg (SEQ ID NO: 497) | gcacaaa (SEQ ID NO: 611) |
| rs6882848 | tcgtcggcagcgtcagatgtgtataagagacagaaagtggtggtttttaaccccttc | gtctcgtgggctcggagatgtgtataagagacagtccttggcagccgttcc (SEQ |
| | (SEQ ID NO: 498) | ID NO: 612) |
| rs1465127 | tcgtcggcagcgtcagatgtgtataagagacaggcctatagatggcaaattaagaga | gtctcgtgggctcggagatgtgtataagagacagaacacacagacaggcaggtt |
| | gca (SEQ ID NO: 499) | (SEQ ID NO: 613) |
| rs1161899 | tcgtcggcagcgtcagatgtgtataagagacagaaaaagtgaatcaatagagtacta | gtctcgtgggctcggagatgtgtataagagacagagtgctcaatagttaccataatgc |
| | gtgcta (SEQ ID NO: 500) | tatattg (SEQ ID NO: 614) |
| rs4615440 | tcgtcggcagcgtcagatgtgtataagagacagatgggaagggtacgatgttacc | gtctcgtgggctcggagatgtgtataagagacagcctcctctctgtgtccatagaac |
| | (SEQ ID NO: 501) | (SEQ ID NO: 615) |
| rs9501710 | tcgtcggcagcgtcagatgtgtataagagacaggattaggataattttccagctcaaa | gtctcgtgggctcggagatgtgtataagagacagtcaatggttttaccatttaaaaattc |
| | gaaaat (SEQ ID NO: 502) | cctatc (SEQ ID NO: 616) |
| rs6925983 | tcgtcggcagcgtcagatgtgtataagagacagcctctaaaactagagtgcctatag | gtctcgtgggctcggagatgtgtataagagacagctcagttgctcagaacaatgtcc |
| | aatttattg (SEQ ID NO: 503) | (SEQ ID NO: 617) |
| rs2972171 | tcgtcggcagcgtcagatgtgtataagagacagagtatttagttaacggttgttttacg | gtctcgtgggctcggagatgtgtataagagacagggagtttcatcaccaagtccaca |
| | ct (SEQ ID NO: 504) | (SEQ ID NO: 618) |
| rs62477557 | tcgtcggcagcgtcagatgtgtataagagacaggaattttgatgaaaacattcctgcta | gtctcgtgggctcggagatgtgtataagagacagcccttgctatcaatattcaaagag |
| | tca (SEQ ID NO: 505) | agaaa (SEQ ID NO: 619) |
| rs4876049 | tcgtcggcagcgtcagatgtgtataagagacagcacagtgttctacggtatacaagta | gtctcgtgggctcggagatgtgtataagagacaggctcgtaggtgtgcaccat |
| | tct (SEQ ID NO: 506) | (SEQ ID NO: 620) |
| rs1509186 | tcgtcggcagcgtcagatgtgtataagagacaggctaccttatagtcttccctagctta | gtctcgtgggctcggagatgtgtataagagacagagaacattcaatgatataaaagg |
| | ataattt (SEQ ID NO: 507) | aataagagaac (SEQ ID NO: 621) |
| rs1876904 | tcgtcggcagcgtcagatgtgtataagagacaggcagggtggctgcgt (SEQ | gtctcgtgggctcggagatgtgtataagagacagtccttggagctgacatggc |
| | ID NO: 508) | (SEQ ID NO: 622) |
| rs4880811 | tcgtcggcagcgtcagatgtgtataagagacaggcttggaatgaaatccctatcccta | gtctcgtgggctcggagatgtgtataagagacaggggatctctcatctcaggcttg |
| | t (SEQ ID NO: 509) | (SEQ ID NO: 623) |
| rs75196694 | tcgtcggcagcgtcagatgtgtataagagacagcagtggtcctgacgttcgg | gtctcgtgggctcggagatgtgtataagagacagtgtgtgccctcgaaccg (SEQ |
| | (SEQ ID NO: 510) | ID NO: 624) |
| rs2075545 | tcgtcggcagcgtcagatgtgtataagagacagcctaatacattaaagcagtcactttt | gtctcgtgggctcggagatgtgtataagagacagcgaccccatctctgagtcct |
| | cct (SEQ ID NO: 511) | (SEQ ID NO: 625) |
| rs60326265 | tcgtcggcagcgtcagatgtgtataagagacagggctcacgtcatgggca (SEQ | gtctcgtgggctcggagatgtgtataagagacagctaggagcagtcaggcttaaga |
| | ID NO: 512) | g (SEQ ID NO: 626) |
| rs953385 | tcgtcggcagcgtcagatgtgtataagagacagagaactcaaacaagatttaaggtc | gtctcgtgggctcggagatgtgtataagagacagtgaagaacatgcttgccatagc |
| | tagaaa (SEQ ID NO: 513) | (SEQ ID NO: 627) |
| rs77983336 | tcgtcggcagcgtcagatgtgtataagagacagcctccactcaaagtttctggc | gtctcgtgggctcggagatgtgtataagagacaggcactattcaggcaaaggctc |
| | (SEQ ID NO: 514) | (SEQ ID NO: 628) |
| rs1547149 | tcgtcggcagcgtcagatgtgtataagagacagtggcacagactttattggctct | gtctcgtgggctcggagatgtgtataagagacagcccagaggattaagagacatgg |
| | (SEQ ID NO: 515) | c (SEQ ID NO: 629) |
| rs3117978 | tcgtcggcagcgtcagatgtgtataagagacagcagagatcatttctattgccacagg | gtctcgtgggctcggagatgtgtataagagacagaagctctagaaaaggcaaaact |
| | (SEQ ID NO: 516) | aaacta (SEQ ID NO: 630) |
| rs9509962 | tcgtcggcagcgtcagatgtgtataagagacaggtaagcctagtgcccagtatatcat | gtctcgtgggctcggagatgtgtataagagacagtctcctattcagcctataagtgttt |
| | (SEQ ID NO: 517) | ctaa (SEQ ID NO: 631) |
| rs7139530 | tcgtcggcagcgtcagatgtgtataagagacaggctagtgtacgatatgtgtgtattg | gtctcgtgggctcggagatgtgtataagagacagaaaacgacttacacatacctaaa |
| | attaa (SEQ ID NO: 518) | atgaaattt (SEQ ID NO: 632) |
| rs292476 | tcgtcggcagcgtcagatgtgtataagagacagaccctcctgcttatgtggttac | gtctcgtgggctcggagatgtgtataagagacagtttgatttgggagcaaagaatga |
| | (SEQ ID NO: 519) | gt (SEQ ID NO: 633) |
| rs3000029 | tcgtcggcagcgtcagatgtgtataagagacagccctgggtcacacacaaca | gtctcgtgggctcggagatgtgtataagagacaggcatctctatgccaaactggtcat |
| | (SEQ ID NO: 520) | a (SEQ ID NO: 634) |
| rs12434992 | tcgtcggcagcgtcagatgtgtataagagacagaagtgagtgggaacagtcatattg | gtctcgtgggctcggagatgtgtataagagacagccaaactacttcttttctaacagaa |
| | a (SEQ ID NO: 521) | agca (SEQ ID NO: 635) |
| rs1760904 | tcgtcggcagcgtcagatgtgtataagagacaggcagataggtacagaggcgtct | gtctcgtgggctcggagatgtgtataagagacaggcatctcttgtgtcagccct |
| | (SEQ ID NO: 522) | (SEQ ID NO: 636) |
| rs35567022 | tcgtcggcagcgtcagatgtgtataagagacagttgactttccagaccccactta | gtctcgtgggctcggagatgtgtataagagacagccagggaaaaaatatgttcgatg |
| | (SEQ ID NO: 523) | cc (SEQ ID NO: 637) |
| rs12910624 | tcgtcggcagcgtcagatgtgtataagagacaggacttgggaagtttttgattactaat | gtctcgtgggctcggagatgtgtataagagacaggataacatagtaatgaatacattt |
| | tcaat (SEQ ID NO: 524) | ctaaaaccgtaa (SEQ ID NO: 638) |
| rs34714665 | tcgtcggcagcgtcagatgtgtataagagacagctttttatatattgcacactctaaaaa | gtctcgtgggctcggagatgtgtataagagacagtccagaagattagttgaaaatttg |
| | gaggt (SEQ ID NO: 525) | agtacaa (SEQ ID NO: 639) |
| rs6576457 | tcgtcggcagcgtcagatgtgtataagagacaggggaaaaacaaaattgtctcaaaa | gtctcgtgggctcggagatgtgtataagagacagcgttttgtcatttttgcagataaatg |
| | aatgt (SEQ ID NO: 526) | tagt (SEQ ID NO: 640) |
| rs2239669 | tcgtcggcagcgtcagatgtgtataagagacaggggcggatgccattgagt (SEQ | gtctcgtgggctcggagatgtgtataagagacagagcaaaaccgcaacccact |
| | ID NO: 527) | (SEQ ID NO: 641) |
| rs1698232 | tcgtcggcagcgtcagatgtgtataagagacaggcacttctaagttattatgatagagt | gtctcgtgggctcggagatgtgtataagagacagactcatatctcccaacacaaaact |
| | gatgtac (SEQ ID NO: 528) | aaaa (SEQ ID NO: 642) |
| rs670962 | tcgtcggcagcgtcagatgtgtataagagacaggcagtaaatcaacccgctataaac | gtctcgtgggctcggagatgtgtataagagacagtgaagcgtgaacttcctcagg |
| | g (SEQ ID NO: 529) | (SEQ ID NO: 643) |
| rs58445115 | tcgtcggcagcgtcagatgtgtataagagacaggagcccttgccaatagtgaaa | gtctcgtgggctcggagatgtgtataagagacagcacctgggaagagaggtgt |
| | (SEQ ID NO: 530) | (SEQ ID NO: 644) |
| rs59061318 | tcgtcggcagcgtcagatgtgtataagagacagcagctagttctatatttacagacag | gtctcgtgggctcggagatgtgtataagagacagtgaatatgtcttcagtgcttagcct |
| | agac (SEQ ID NO: 531) | (SEQ ID NO: 645) |
| rs6506015 | tcgtcggcagcgtcagatgtgtataagagacagaatcctgtatctagtgccaatctag | gtctcgtgggctcggagatgtgtataagagacagcctaaaaatcgttacttctcctttat |
| | aa (SEQ ID NO: 532) | tttttttc (SEQ ID NO: 646) |
| rs72634353 | tcgtcggcagcgtcagatgtgtataagagacagagtatctataatagtgcgtggcaca | gtctcgtgggctcggagatgtgtataagagacaggcctataacaatgtactagaacc |
| | ta (SEQ ID NO: 533) | aagtattt (SEQ ID NO: 647) |
| rs55677929 | tcgtcggcagcgtcagatgtgtataagagacagggaagacccggcggga (SEQ | gtctcgtgggctcggagatgtgtataagagacaggggtgtagggcaggggt |
| | ID NO: 534) | (SEQ ID NO: 648) |
| rs6135141 | tcgtcggcagcgtcagatgtgtataagagacagtcctctcctgcttaatgtagtcac | gtctcgtgggctcggagatgtgtataagagacagttcacaagcagagttgaaagact |
| | (SEQ ID NO: 535) | (SEQ ID NO: 649) |
| rs2050980 | tcgtcggcagcgtcagatgtgtataagagacagatccaccagagaatacacaaatta | gtctcgtgggctcggagatgtgtataagagacagaaaagactgtcagtgatatcttag |
| | tatgtatatat (SEQ ID NO: 536) | gtaga (SEQ ID NO: 650) |
| rs4815580 | tcgtcggcagcgtcagatgtgtataagagacagggtacatgaccataataaatcagc | gtctcgtgggctcggagatgtgtataagagacagggccaacattttgttttataatctg |
| | agg (SEQ ID NO: 537) | cg (SEQ ID NO: 651) |
| rs463397 | tcgtcggcagcgtcagatgtgtataagagacagctctctctactgaattttgattttccatttc | gtctcgtgggctcggagatgtgtataagagacagctaggatcaaagaagaatagaa |
| | (SEQ ID NO: 538) | aaagtggt (SEQ ID NO: 652) |
| rs7279689 | tcgtcggcagcgtcagatgtgtataagagacagacactgagtattcccaatgtaaag | gtctcgtgggctcggagatgtgtataagagacagacaataattgtacttatttatggag |
| | aaataat (SEQ ID NO: 539) | tacatagtgat (SEQ ID NO: 653) |
| rs5748211 | tcgtcggcagcgtcagatgtgtataagagacagcctgggcatcgccct (SEQ | gtctcgtgggctcggagatgtgtataagagacagcctaggaggtgacctcactaaa |
| | ID NO: 540) | at (SEQ ID NO: 654) |
| rs79114187 | tcgtcggcagcgtcagatgtgtataagagacagggcccactgcactcacct (SEQ | gtctcgtgggctcggagatgtgtataagagacagactatctacatcagtgcgagaga |
| | ID NO: 541) | aag (SEQ ID NO: 655) |
| rs13164 | tcgtcggcagcgtcagatgtgtataagagacagcaggtagggaaagatttcttaagt | gtctcgtgggctcggagatgtgtataagagacagtgtgcagagtcccccagg |
| | gag (SEQ ID NO: 542) | (SEQ ID NO: 656) |
| rs4633 | tcgtcggcagcgtcagatgtgtataagagacagcctgcagcccatccacaac | gtctcgtgggctcggagatgtgtataagagacagggcctccagcacgctc (SEQ |
| | (SEQ ID NO: 543) | ID NO: 657) |
| rs13303106 | tcgtcggcagcgtcagatgtgtataagagacagactgtgagaggctcagaagga | gtctcgtgggctcggagatgtgtataagagacaggggtagattccaggggctct |
| | (SEQ ID NO: 544) | (SEQ ID NO: 658) |
| rs35273536 | tcgtcggcagcgtcagatgtgtataagagacagggggtcagcaggtggca (SEQ | gtctcgtgggctcggagatgtgtataagagacagggaacactatctgaaatagaccc |
| | ID NO: 545) | tcg (SEQ ID NO: 659) |
| rs77129670 | tcgtcggcagcgtcagatgtgtataagagacaggggagatgaaataagtaccaaaa | gtctcgtgggctcggagatgtgtataagagacagcctgcaggtctttccgattctg |
| | tgagt (SEQ ID NO: 546) | (SEQ ID NO: 660) |
| rs17133064 | tcgtcggcagcgtcagatgtgtataagagacaggcattgccactttggctttcg | gtctcgtgggctcggagatgtgtataagagacaggattttaaaaccagagataattct |
| | (SEQ ID NO: 547) | gaaaggaa (SEQ ID NO: 661) |
| rs1161901 | tcgtcggcagcgtcagatgtgtataagagacagcctctctaaaacttgatgattttaac | gtctcgtgggctcggagatgtgtataagagacaggctggtcctcactgacatcc |
| | atgtaat (SEQ ID NO: 548) | (SEQ ID NO: 662) |
| rs77474447 | tcgtcggcagcgtcagatgtgtataagagacaggacccacagccgtggt (SEQ | gtctcgtgggctcggagatgtgtataagagacagtctaaccccgtcatgctgc |
| | ID NO: 549) | (SEQ ID NO: 663) |
| rs17756915 | tcgtcggcagcgtcagatgtgtataagagacagaaatatatagagccgcacaccaa | gtctcgtgggctcggagatgtgtataagagacaggtgcaatgttaactttattaattagt |
| | aaata (SEQ ID NO: 550) | tgacttc (SEQ ID NO: 664) |
| rs341697 | tcgtcggcagcgtcagatgtgtataagagacagaaaaatggggcagaatgttgtca | gtctcgtgggctcggagatgtgtataagagacagttgctgttccacaaaacatagctt |
| | (SEQ ID NO: 551) | (SEQ ID NO: 665) |
| rs10976019 | tcgtcggcagcgtcagatgtgtataagagacaggagcaagttcggtctggct | gtctcgtgggctcggagatgtgtataagagacaggctgattaattaggtgtttgttagc |
| | (SEQ ID NO: 552) | ag (SEQ ID NO: 666) |
| rs76408959 | tcgtcggcagcgtcagatgtgtataagagacagcccattgattaaacaaatattcact | gtctcgtgggctcggagatgtgtataagagacagcctcaccctccatccctca |
| | gagtac (SEQ ID NO: 553) | (SEQ ID NO: 667) |
| rs9734804 | tcgtcggcagcgtcagatgtgtataagagacagcttcgggcctctggacc (SEQ | gtctcgtgggctcggagatgtgtataagagacagcccaaggcgggcacct (SEQ |
| | ID NO: 554) | ID NO: 668) |
| rs12792188 | tcgtcggcagcgtcagatgtgtataagagacagatctcccgtctcatcctgaaac | gtctcgtgggctcggagatgtgtataagagacaggggtgctgcgcccaga (SEQ |
| | (SEQ ID NO: 555) | ID NO: 669) |
| rs11611246 | tcgtcggcagcgtcagatgtgtataagagacaggtggaaggcatactgagtgaact | gtctcgtgggctcggagatgtgtataagagacagggataaactagtggttctttgatct |
| | (SEQ ID NO: 556) | ttatcttt (SEQ ID NO: 670) |
| rs79782920 | tcgtcggcagcgtcagatgtgtataagagacaggtgtctccattacattgcttgttttaattt | gtctcgtgggctcggagatgtgtataagagacagaacatattgaacttatttttaaaag |
| | (SEQ ID NO: 557) | ggggag (SEQ ID NO: 671) |
| rs7989876 | tcgtcggcagcgtcagatgtgtataagagacagtctgacatttcacagctggca | gtctcgtgggctcggagatgtgtataagagacaggggggatctgccatacagc |
| | (SEQ ID NO: 558) | (SEQ ID NO: 672) |
| rs7982082 | tcgtcggcagcgtcagatgtgtataagagacaggctcccaccagctactgtga | gtctcgtgggctcggagatgtgtataagagacagtcaagccttgacttttaaagcaca |
| | (SEQ ID NO: 559) | (SEQ ID NO: 673) |
| rs77905703 | tcgtcggcagcgtcagatgtgtataagagacaggttcacccagaagtcattccgta | gtctcgtgggctcggagatgtgtataagagacagttgttgttgtcagcttagaatagtat |
| | (SEQ ID NO: 560) | atcatat (SEQ ID NO: 674) |
| rs59329234 | tcgtcggcagcgtcagatgtgtataagagacaggaggcacagtgctttgtatttgat | gtctcgtgggctcggagatgtgtataagagacagcccttttataatcttgttacttttatgt |
| | (SEQ ID NO: 561) | tgaact (SEQ ID NO: 675) |
| rs150926 | tcgtcggcagcgtcagatgtgtataagagacagttagccactacctttttggctacta | gtctcgtgggctcggagatgtgtataagagacagggggagagaaaaccgtatgtga |
| | (SEQ ID NO: 562) | t (SEQ ID NO: 676) |
| rs12450330 | tcgtcggcagcgtcagatgtgtataagagacagcgttttacagcaagcgacagaa | gtctcgtgggctcggagatgtgtataagagacagttttagatctctttcagttttggtttgc |
| | (SEQ ID NO: 563) | (SEQ ID NO: 677) |
| rs16948415 | tcgtcggcagcgtcagatgtgtataagagacagatgtgatgtgctctaggaaaatgc | gtctcgtgggctcggagatgtgtataagagacagggagttataaaaaagaacagaa |
| | (SEQ ID NO: 564) | ggtcatgat (SEQ ID NO: 678) |
| rs11878153 | tcgtcggcagcgtcagatgtgtataagagacaggccttgtgagaacagactaataca | gtctcgtgggctcggagatgtgtataagagacaggcctccttcactcgcagt (SEQ |
| | gata (SEQ ID NO: 565) | ID NO: 679) |
| rs2279796 | tcgtcggcagcgtcagatgtgtataagagacagggtaggtgtggtcaggtcga | gtctcgtgggctcggagatgtgtataagagacaggcacaagcggtcaacagc |
| | (SEQ ID NO: 566) | (SEQ ID NO: 680) |
| rs6074167 | tcgtcggcagcgtcagatgtgtataagagacagacctccctcttgggatgca | gtctcgtgggctcggagatgtgtataagagacaggggtttatcaaaaggtttgctgc |
| | (SEQ ID NO: 567) | (SEQ ID NO: 681) |
| rs2823170 | tcgtcggcagcgtcagatgtgtataagagacagggagaataatagttaattaatccac | gtctcgtgggctcggagatgtgtataagagacagcgctaaaaaaggaagaactagg |
| | gaagca (SEQ ID NO: 568) | aaagat (SEQ ID NO: 682) |
| rs9984697 | tcgtcggcagcgtcagatgtgtataagagacagagagtctcaattattgctcagttag | gtctcgtgggctcggagatgtgtataagagacagaatatctatccagagtatcgatta |
| | gat (SEQ ID NO: 569) | atcttcataaaa (SEQ ID NO: 683) |
| rs17809319 | tcgtcggcagcgtcagatgtgtataagagacagggtgacctgcgttacttgcttat | gtctcgtgggctcggagatgtgtataagagacagacctagtacgtcatttataatgaa |
| | (SEQ ID NO: 570) | aattgc (SEQ ID NO: 684) |

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
U.S. Pat. No. 4,683,195
U.S. Pat. No. 4,683,202
U.S. Pat. No. 4,800,159
U.S. Pat. No. 6,664,079
U.S. Pat. No. 8,612,161
U.S. Pat. No. 8,623,598
U.S. Pat. No. 9,284,602
U.S. Pat. Publn. No. 2009/0026082
U.S. Pat. Publn. No. 2010/0137143
U.S. Pat. Publn. No. 2010/0282617
U.S. Pat. Publn. No. 2016/0326600
U.S. Pat. Publn. No. 2016/0340727
U.S. Pat. Publn. No. 2017/0029875
Margulies et al., Nature, 437:376-380, 2005.
McPherson et al., editors, PCR: A Practical Approach (IRL Press, Oxford, 1991).
McPherson et al., editors, PCR2: A Practical Approach (IRL Press, Oxford, 1995).
Oyola et al., BMC Genomics, 13:1, 2012.
Pareek et al., Sequencing technologies and genome sequencing, J. Appl. Genet., 52(4):413-435, 2011.
Thudi et al., Current state-of-art of sequencing technologies for plant genomics research, Brief Funct. Genomics, 11(1):3-11, 2012.
Wang et al., Modular probes for enriching and detecting complex nucleic acid sequences, Nature Chemistry, DOI: 10.1038/NCHEM.2820, Published online Jul. 17, 2017.
Wang and Zhang, Simulation-guided DNA probe design for consistently ultraspecific hybridization, Nature Chemistry, 7:545-53, 2015.
Wu et al., Continuously tunable nucleic acid hybridization probes, Nature Methods, 12:1191-96, 2015.
Zhang et al., Optimizing the specificity of nucleic acid hybridization, Nature Chemistry, 4:208-14, 2012.

What is claimed is:

1. A method of detecting the presence of rare sequence variants within a DNA region of interest, the method comprising:
(a) amplifying one or more region of interest using polymerase chain reaction (PCR) with primers, each primer comprising a 5′ sequence-adaptor region and a 3′ gene-specific region, thereby generating double-stranded amplicons;
(b) denaturing the double-stranded amplicons, thereby generating single-stranded amplicons;
(c) hybridizing the single-stranded amplicons to a mixture of negative-selection Sinks;
(d) removing the single-stranded amplicons bound to Sinks;
(e) amplifying the remaining single-stranded amplicons by PCR using primers comprising sequencing adaptor sequences; and
(f) performing high-throughput DNA sequencing.

2. The method of claim 1, wherein the rare variant is of unknown sequence identity.

3. The method of claim 1, wherein the rare variant is of known sequence identity.

4. The method of claim 3, wherein step (c) further comprises hybridizing the single-stranded amplicons to a mixture of positive-selection Probes.

5. The method of claim 4, wherein the Probes comprise toehold probes, fine-tuned probes, or X-probes.

6. The method of either claim 3 or 4, wherein the Probes and Sinks are thermodynamically competitive.

7. The method of any one of claims 3-6, wherein there is one Probe and one Sink for each rare sequence variant.

8. The method of any one of claims 3-6, wherein there is one Probe for each rare sequence variant.

9. The method of any one of claims 3-6, wherein the Probes comprise Probes having paired probe complement and probe protector oligonucleotides of Table 1.

10. The method of any one of claims 1-9, wherein the Sinks comprise Sinks having paired sink complement and sink protector oligonucleotides of Table 2.

11. The method of either claim 3 or 4, wherein step (d) further comprises collecting amplicons bound to Probes.

12. The method of claim 11, wherein step (d) is performed via streptavidin-coated magnetic beads, collecting is performed using a magnet, and the Probes in step (c) are either directly functionalized with a biotin or hybridized to a universal oligonucleotide functionalized with a biotin.

13. The method of claim 11, wherein step (d) is performed via streptavidin-coated agarose beads, collection is performed using centrifugal force, and the Probes in step (c) are either directly functionalized with a biotin or hybridized to a universal oligonucleotide functionalized with a biotin.

14. The method of any one of claims 11-13, wherein removing the single-stranded amplicons bound to Sinks occurs by way of collecting amplicons bound to Probes.

15. The method of any one of claims 1-14, wherein the hybridization in step (c) is performed at a temperature of between about 15° C. and about 75° C.

16. The method of any one of claims 1-15, wherein the hybridization in step (c) is performed in a buffer with a monovalent cation concentration of between about 50 mM and about 5 M.

17. The method of claim 16, wherein the monovalent cation is sodium.

18. The method of any one of claim 1-15, wherein the hybridization in step (c) is performed in a buffer with a divalent cation concentration of between about 3 mM and about 30 mM.

19. The method of claim 18, wherein the divalent cation is magnesium.

20. The method of any one of claims 1-14, wherein the PCR of step (a) is multiplex PCR when amplifying more than one region of interest.

21. The method of any one of claims 1-20, wherein the PCR of step (a) is carried out for 4-20 cycles.

22. The method of any one of claims 1-20, wherein the PCR of step (a) is carried out for no more than 20 cycles.

23. The method of any one of claims 1-22, wherein step (b) is performed via heat denaturation.

24. The method of claim 23, wherein heat denaturation comprises heating the amplicon mixture to at least 80° C. for at least 2 minutes.

25. The method of any one of claims 1-24, wherein step (b) is performed via DNAse activity and wherein one of the primers in step (a) is modified with either a 5′ phosphate functionalization to encourage degradation or a 5′ functionalization to inhibit degradation.

26. The method of claim 25, wherein the 5′ primer functionalization comprises a phosphorothioate, a 2′-O-methyl group, or a non-natural nucleotide.

27. The method of any one of claims 1-26, wherein the Sinks in step (c) comprise toehold probes, fine-tuned probes, or X-probes.

28. The method of any one of claims 1-27, wherein the removing in step (d) is performed via solid-phase separation.

29. The method of any one of claims 1-11 and 20-28, wherein step (d) is performed via streptavidin-coated magnetic beads, removing is performed using a magnet, and the Sinks in step (c) are either directly functionalized with a biotin or hybridized to a universal oligonucleotide functionalized with a biotin.

30. The method of any one of claims 1-11 and 20-28, wherein step (d) is performed via streptavidin-coated agarose beads, removing is performed using centrifugal force, and the Sinks in step (c) are either directly functionalized with a biotin or hybridized to a universal oligonucleotide functionalized with a biotin.

31. The method of any one of claims 1-30, wherein the primers in step (e) are universal primers.

32. The method of any one of claims 1-31, wherein the primers in step (e) further comprise a sample barcode or index sequence.

33. The method of any one of claims 1-32, wherein the sequencing in step (f) is sequencing-by-synthesis.

34. The method of any one of claims 1-32, wherein the sequencing in step (f) is nanopore sequencing.

35. The method of any one of claims 1-32, wherein the sequencing in step (f) is sequencing-by-hybridization.

36. The method of any one of claims 1-35, further comprising (g) analyzing the DNA sequencing data to calculate the ratio of reads observed for variant sequences as compared to wild-type sequences.

37. The method of claim 33, wherein the sequencing in step (f) is paired-end sequencing.

38. The method of claim 36, wherein the analysis in step (g) does not consider any sequencing read in which the forward read and the reverse read do not perfectly agree on the sequence of the amplicon insert.

39. The method of claim 36, wherein the analysis in step (g) does not consider any sequencing reading in which a read quality score is below 30.

40. The method of any one of claims 1-39, further defined as a method of quantifying the presence of rare sequence variants within a DNA region of interest.
